(19) United States
(12) Patent Application Publication
(10) Pub. No.: US 2025/0115874 A1
(43) Pub. Date: Apr. 10, 2025

(54) Compositions And Methods For Making And Using Atherosclerotic Nanomatrix Vascular Sheets

(71) Applicant: UAB RESEARCH FOUNDATION, Birmingham, AL (US)

(72) Inventors: Jun Chen, Birmingham, AL (US); Xixi Zhang, Birmingham, AL (US); Brigitta C. Brott, Birmingham, AL (US); Ho-Wook Jun, Birmingham, AL (US)

(21) Appl. No.: 18/482,869

(22) Filed: Oct. 7, 2023

(51) Int. Cl. C12N 5/077; C07K 14/00; C12N 5/071

(52) U.S. Cl. CPC C12N 5/0656; C07K 14/001; C12N 5/0661; C12N 5/069; C12N 2501/22; C12N 2501/24; C12N 2501/25; C12N 2501/999; C12N 2502/1157; C12N 2537/10

(57) ABSTRACT

Disclosed are nanomatrix vascular sheets comprising a nanomatrix fibroblast sheet comprising peptide amphiphiles and fibroblasts, a nanomatrix smooth muscle cell sheet comprising peptide amphiphiles and smooth muscle cells, and a layer of endothelial cells. Disclosed are methods of producing a nanomatrix vascular sheet comprising combining fibroblast cells with a peptide amphiphile solution and crosslinking the peptide amphiphiles in the nanomatrix fibroblast sheet, thereby forming a nanomatrix fibroblast sheet; seeding fibroblasts on the Nanomatrix fibroblast sheet; producing a Nanomatrix smooth muscle cell sheet on top of the seeded fibroblasts, thereby forming a double-layer nanomatrix cell sheet; seeding smooth muscle cells on the double-layer Nanomatrix cell sheet; and seeding endothelial cells on top of the seeded smooth muscle cells, thereby forming a trilayer Nanomatrix cell sheet.

### STATEMENT REGARDING FEDERALLY SPONSORED RESEARCH

This invention was made with government support under 1R01HL163802-01 awarded by the National Institutes of Health. The government has certain rights in the invention.

### REFERENCE TO SEQUENCE LISTING

The Sequence Listing submitted Feb. 20, 2024 as an xml file named “21085.0199U1.xml,” created on Feb. 20, 2024, and having a size of 4,512 bytes is hereby incorporated by reference pursuant to 37 C.F.R. § 1.52(e)(5).

### BACKGROUND

Cardiovascular diseases (CVDs) are the leading cause of death globally. CVDs primarily result from atherosclerosis, dyslipidemia, and inflammation-driven pathophysiological process, where the arteries are narrowed and hardened due to the formation of atherosclerotic plaque within the arterial walls. Atherosclerotic plaques are detrimental to human physiology, leading to severe health issues such as stroke, myocardial infarction, or death. Thus, there is a large and growing need for therapeutics targeting atherosclerosis.

During the development of effective therapies for atherosclerosis, in vitro models are commonly used to evaluate the efficacy and safety of novel therapeutics prior to initiating complicated in vivo and clinical studies. Compared to in vivo models, in vitro models provide human-relevant data in a cheaper, more controllable, and higher-throughput manner with improved reproducibility. Current in vitro models are generally classified into two-dimensional (2D) and three-dimensional (3D) models. Traditional in vitro 2D models include cultures of single or multiple vascular cell types on tissue culture plates (TCP), which have been widely used to assess drug safety and efficacy. Still, these models are limited to a 2D environment, and their cellular compositions are too simple to mimic atherosclerotic vessel physiology closely. Thus, data generated using those models may result in misleading predictions regarding the efficacy of therapeutic candidates. With recent advances in tissue engineering, biomaterial development, and microfluidic approaches, considerable research has been conducted on developing in vitro 3D culture systems to model atherosclerosis. Compared with the 2D culture systems, the engineered 3D culture systems better recapitulate physiological architecture and pathological conditions observed in vivo, which is, therefore, expected to provide more accurate and physiologically relevant systems for predicting and/or testing the pharmacokinetics and pharmacodynamic responses associated with pharmacological agents. However, despite that, current in vitro 3D atherosclerosis platforms or therapeutic drug screening for atherosclerosis studies possess limitations, including high cost, long maturation time, complicated fabrication processes, difficulties in scale-up production, and batch-to-batch variability. Moreover, those 3D systems fail to mimic the three-layer vessel structure and only provide limited atherosclerosis features. Therefore, it is imperative to develop an advanced in vitro 3D atherosclerosis model that possesses a three-layer vascular structure, recapitulates critical multi-features in atherosclerosis, and embraces the strengths of being cost-effective, reproducible, and scalable, improving drug discovery and development process for atherosclerosis.

Recently, there has been increasing interest in designing high-throughput screening (HTS) assays using in vitro models for rapidly generating and analyzing large-scale human-relevant data, accelerating pre-clinical drug evaluation. Reportedly, HTS assays for diseases involve establishing a high-throughput culture system in a multi-well microtiter plate, adding individual drug candidates from a library into each well, and performing biological assays to detect the action of the drug candidate. However, despite the significant progress in this field, up to date, an extensive collection of studies has merely focused on creating HTS assays for cancer research, and very few studies reported on the development of HTS assays associated with atherosclerosis, possibly due to the challenges of emulating atherosclerosis resulting from its complicated pathogenesis. More importantly, in these studies, the HTS assays were generated relying on simple 2D high-throughput culture systems comprising only a single type of cells, and, in some cases, using non-vascular cells (HepG2 or COS-7), which may lead to inaccurate drug efficacy and safety prediction. Therefore, to tackle these issues and consider the critical roles of cell-cell communication and cell-ECM interaction in the atherosclerosis environment, the next generation of HTS assays should be created using more advanced 3D atherosclerosis systems with multiple vascular cells.

there remains a need for an improved in vitro 3D vascular tissue model and atherosclerosis model for generating HTSs to provide better opportunities to obtain meaningful pre-clinical information.

### BRIEF SUMMARY

Disclosed are nanomatrix vascular sheets comprising a first layer, wherein the first layer is a nanomatrix cell sheet having fibroblasts, a second layer, wherein the second layer is a nanomatrix cell sheet having smooth muscle cells (SMCs), and a third layer, wherein the third layer comprises endothelial cells (ECs).

Disclosed are nanomatrix vascular sheets comprising a first layer, wherein the first layer is a nanomatrix cell sheet having fibroblasts; a second layer, wherein the second layer comprises seeded fibroblasts or SMCs; a third layer, wherein the third layer is a nanomatrix cell sheet having SMCs; a fourth layer, wherein the fourth layer comprises seeded SMCs; and a fifth layer, wherein the fifth layer comprises ECs. Disclosed are atherosclerotic nanomatrix vascular sheets comprising a first layer, wherein the first layer is a nanomatrix cell sheet having fibroblasts, a second layer, wherein the second layer is a nanomatrix cell sheet having smooth muscle cells (SMCs), and a third layer, wherein the third layer comprises endothelial cells (ECs), wherein the endothelial cells are dysfunctional, wherein the fibroblasts are inflammation activated fibroblasts, wherein the SMCs are inflammation activated SMCs, wherein the atherosclerotic nanomatrix vascular sheet comprises one or more of monocytes, macrophages, foam cells, low-density lipoproteins, cytokines, chemokines, reactive oxygen species, or calcification.

Disclosed are methods of producing a single-layer nanomatrix fibroblast sheet. In some aspects, are methods of producing a nanomatrix fibroblast sheet comprise combining fibroblast cells with a peptide amphiphile solution, crosslinking the peptide amphiphiles thereby forming a nanomatrix containing fibroblasts, and culturing the nanomatrix containing fibroblasts in a culture medium, thereby forming a nanomatrix fibroblast sheet.

Disclosed are methods of producing a nanomatrix SMC sheet comprise combining SMC cells with a peptide amphiphile solution, crosslinking the peptide amphiphiles, thereby forming a Nanomatrix containing SMCs, and culturing the nanomatrix containing SMCs in a culture medium, thereby forming a nanomatrix SMC sheet.

Disclosed are methods of producing a double-layer nanomatrix cell sheet comprising combining fibroblast cells with a peptide amphiphile solution, crosslinking the peptide amphiphiles in the nanomatrix fibroblast sheet, thereby forming a Nanomatrix containing fibroblasts, and culturing the Nanomatrix containing fibroblasts in a culture medium, thereby forming a nanomatrix fibroblast sheet, seeding fibroblasts or SMCs on a nanomatrix fibroblast sheet, and producing a nanomatrix SMC sheet on top of the seeded fibroblasts or SMCs, and followed by culturing to form a double-layer nanomatrix cell sheet.

Disclosed are methods of producing a nanomatrix vascular sheet comprising combining fibroblast cells with a peptide amphiphile solution and crosslinking the peptide amphiphiles in the nanomatrix fibroblast sheet, thereby forming a nanomatrix fibroblast sheet; seeding fibroblasts on the Nanomatrix fibroblast sheet; producing a Nanomatrix SMC sheet on top of the seeded fibroblasts, thereby forming a double-layer nanomatrix cell sheet; seeding SMCs on the double-layer nanomatrix cell sheet; and seeding endothelial cells on top of the seeded SMCs, thereby forming a trilayer nanomatrix cell sheet. In some aspects, the method further comprises inducing endothelial cell dysfunction which leads to inducing monocyte recruitment, inducing macrophage formation, inducing foam cell formation, and thus forming an atherosclerotic nanomatrix vascular sheet.

Disclosed are methods of screening for a therapeutic drug candidate comprising culturing an atherosclerotic nanomatrix vascular sheet in the presence of the therapeutic drug candidate; culturing an atherosclerotic nanomatrix vascular sheet in the absence of the therapeutic drug candidate; and detecting the presence of lipids, quantifying pro-inflammatory mediators, or both in the atherosclerotic nanomatrix vascular sheet cultured in the presence and absence of the therapeutic drug candidate; wherein fewer lipids, less pro-inflammatory mediators, or both in the atherosclerotic nanomatrix vascular sheet cultured in the presence of the therapeutic drug candidate compared to the atherosclerotic nanomatrix vascular sheet cultured in the absence of the therapeutic drug candidate indicates the therapeutic drug candidate is effective at treating atherosclerosis.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A shows a schematic diagram depicting the steps of fabricating single-layer nanomatrix VS, including single-layer nanomatrix vascular hAAF, hAoSMC, or hAEC sheet.

FIG. 1B shows a representative top-down view image of single-layer nanomatrix hAAF, hAoSMC, or hAEC sheet (2 cm×2 cm) after 1- or 7-day culture. FIG. 1C shows the representative bright-field image of a single-layer nanomatrix hAAF sheet and fluorescent images of a single-layer nanomatrix hAAF sheet with live(green)/dead(red) staining and S100A4 immunostaining for indicating cell viability and phenotype, respectively. FIG. 1D shows the representative bright-field image of single-layer nanomatrix hAoSMC sheet and the fluorescent images of single-layer nanomatrix hAoSMC sheet with live (green)/dead (red) staining and SMA-α immunostaining (red) for indicating cell viability and phenotype, respectively. FIG. 1E shows the representative bright-field image of single-layer nanomatrix hAEC sheet and the fluorescent images of single-layer nanomatrix hAEC sheet with live (green)/dead (red) or DAF-FM staining (green) for indicating cell viability and endothelial function, respectively.

FIG. 2A illustrates the detailed steps for developing a double-layer nanomatrix vascular sheet. FIG. 2B shows the representative top-down image of the double-layer nanomatrix vascular sheet (hAoSMC-hAAF sheet) on day 1 and day 7, with 1 million hAAFs/cm squares as the glue, for showing double-layer nanomatrix vascular sheet formation. FIG. 2C shows representative fluorescent images of the double-layer cell sheet, hAoSMC-hAAF sheet, with live (green)/dead (red) staining and S100A4 (green) or SMA-α (red) immunostaining for showing the cell viability and phenotype in the double-layer nanomatrix vascular sheet, respectively.

FIGS. 3A-3D show the development of a three-layer nanomatrix VS. FIG. 3A depicts the three-layer structure of the nanomatrix vascular sheet (VS) that composes hAEC, hAoSMC, and hAAF layers, which mimics the layered structure of the native vascular wall composed of tunica intima, media, and adventitia. FIG. 3B illustrates the detailed steps for fabricating a three-layer nanomatrix vascular sheet. FIG. 3C shows the representative fluorescent images demonstrating a cross-sectional view of the GFP-hAEC layer (green) and RFP-hAoSMC middle layer (red) of the three-layer nanomatrix vascular sheet and a representative image merged from fluorescent and optical images of the three-layer nanomatrix vascular sheet for showing the three-layer structure of the vascular sheet. FIG. 3D shows the representative fluorescent image of GFP-hAEC layer of the three-layer nanomatrix vascular sheet (GFP-hAEC-GFP-hAoSMC-hAAF sheet) on day 2 and day 7 after GFP-hAEC seeding for showing the cell morphology of the hAEC layer, and with CD31 immunostaining of hAEC layer of the three-layer nanomatrix vascular sheet (hAEC-hAoSMC-hAAF sheet) on day 7 after hAEC seeding for showing cell phenotype of the hAEC layer.

FIGS. 4A-4D show the process of inducing atherosclerosis on the three-layer VS. FIGS. 4A and 4B show the process of the induction of endothelial dysfunction and monocyte recruitment on the three-layer nanomatrix VS, respectively. FIGS. 4C and 4D show the process of induction of macrophage formation, foam cell generation, and calcification initiation.

FIGS. 5A-5D demonstrate the endothelial dysfunction and monocyte recruitment on the three-layer nanomatrix VS induced by the procedure in FIGS. 4A and 4B. FIG. 5A show representative fluorescent images of Ox-LDL and TNF-α untreated (control) and treated three-layer nanomatrix vascular sheet with ICAM-1 immunostaining and DAPI staining for showing successful induction of endothelial dysfunction. FIG. 5B shows the representative fluorescent images of Ox-LDL and TNF-α untreated and treated three-layer nanomatrix vascular sheet with VCAM-1 immunostaining (red) and DAPI staining (blue) for showing successful induction of endothelial dysfunction. FIG. 5C shows the representative fluorescent images of calcein-blue stained monocytes (blue) on the GFP-hAEC layer (green) of Ox-LDL and TNF-α untreated (control) and treated three-layer nanomatrix vascular sheet for showing successful induction of monocyte recruitment. FIG. 5D shows the representative fluorescent images of the cross-section view of the GFP-hAEC layer (green) and RFP-hAoSMC layer (red) of and monocytes (blue) on the Ox-LDL and TNF-α treated three-layer nanomatrix vascular sheet for showing successful induction of monocyte recruitment.

FIGS. 6A-6E demonstrates the induction of macrophage formation and foam cell generation on the three-layer nanomatrix VS. FIG. 6A show the representative fluorescent images of CD14 immunostaining (green) and C68 immunostaining (red) of the VSA for showing monocyte recruitment and macrophage formation. FIG. 6B show the representative fluorescent images of foam cells with BODIPY staining (green) on the sheet for showing the foam cell, lipid pool, and foam cell aggregation (fatty streak). The arrows indicate the single foam cell on VSA.

FIG. 6C show the Quantitative data of BODIPY staining of the VSA (n=5 biological replicates) and control (n=5 biological replicates) for showing the foam cell generation. Bars and error bars represent the mean±s.e.m. e, Inflammatory cytokine secretion by the VSA (n=5 biological replicates) compared with control (VS) (n=5 biological replicates). Bars and error bars represent the mean±s.e.m. FIG. 6D show the quantitative data of oxidative level of VSA using DCFH-DA assay (n=5 biological replicates). mean±s.e.m. FIG. 6E show the alizarin red staining of the VSA (n=5 biological replicates) and VS (n=5 biological replicates) for showing calcification initiation on the VSA. h, Atherosclerotic gene expression of the VSA (n=5 biological replicates) compared with controls (n=5 biological replicates). Bars and error bars represent the mean±s.e.m. The gene expression of endothelial function marker (PECAM-1, eNOS), SMC phenotype markers (MYH11, PDGF-B, NOX4), ROS production (NOX4 and CDKN2A), Calcification (RUNX2, TGF-10), ECM remodeling (MMP2, COL3A1)

FIGS. 7A-7C demonstrates the development and use of VSA high-throughput functional assays for drug evaluation. FIG. 7A demonstrates first step, comprising 1) fabrication of the VSA high-throughput culture system by a) fabricating 3-layer VSs in two 48 well-plates with 96 VSs followed by b) induction of critical atherosclerosis multi-feature in the VS by treating the 96 VSs with an atherosclerosis-inducing medium and monocytes to form VSAs. FIG. 7B demonstrates the second step, a) dividing the 96 VSAs into two groups, group 1 containing 48 VSAs in one plate for foam cell assays, and group 2 containing 48 VSAs in another plate for inflammation assay; b) further dividing the 48 VSAs in group 1 or group 2 into two subgroups, subgroup 1 containing 8 VSAs for controls and subgroup 2 containing 40 VSAs for drug testing; c) treating the 40 VSAs in each group with therapeutics of interest in each group; d) culturing VSAs in both group 1 and group 2 with an atherosclerosis-inducing medium containing monocytes for 10 days. FIG. 7C demonstrates the third step, comprising a) treating the 48 VSAs in group 1 in the plate with BODIPY staining followed by quantifying the fluorescent intensity from BODIPY using a microplate reader to study therapeutic effect on the foam cell formation; b) collecting the medium supernatant from the 48 VSAs in group 2 followed by running the ELISA using a microplate reader to study therapeutic effect on the inflammation; c) collecting the VSAs and conducting PCR to study the effect the therapeutics on atherosclerosis-related gene expression in genetic level. FIG. 7D demonstrates the fourth step, comprising conducting the data analysis.

FIGS. 8A-8D demonstrates the outcomes of evaluating rosuvastatin and sirolimus using VSA high-throughput functional assays. FIG. 8A shows the top-down view of VSA culture systems in the 48 well plate and a single VSA. FIG. 8B shows the quantitative data of BODIPY staining of VSA treated with or without statin or sirolimus for showing the effect of statin or sirolimus on foam cell generation. Bars and error bars represent the mean±s.e.m. (n=5 biological replicates). FIG. 8C shows the quantification of atherosclerosis-related gene expression of VSAs treated with or without statin or sirolimus using qRT-PCR to show the therapeutic effect on atherosclerosis on the genetic level. Bars and error bars represent the mean±s.e.m. (n=5 biological replicates). FIG. 8D shows the quantification of cytokine secretion of VSAs treated with or without statin and sirolimus using ELISA to show the therapeutic effect on atherosclerosis-induced inflammation on the molecular level.

FIGS. 9A-9C demonstrates the outcomes of evaluating curcumin and colchicine using VSA high-throughput functional assays. FIG. 9A demonstrates the quantitative data of foam cells on the VSA treated with or without curcumin or colchicine using BODIPY staining to show the effect of statin or sirolimus on foam cell generation. Bars and error bars represent the mean±s.e.m. (n=5 biological replicates). FIG. 9B demonstrates the quantification of atherosclerosis-related gene expression of VSAs treated with or without curcumin or colchicine using qRT-PCR to show the therapeutic effect on atherosclerosis on the genetic level. Bars and error bars represent the mean±s.e.m. (n=5 biological replicates). FIG. 9C shows the quantification of cytokine secretion of VSAs treated with or without curcumin or colchicine using ELISA to show the therapeutic effect on atherosclerosis-induced inflammation on the molecular level. Bars and error bars represent the mean±s.e.m. (n=5 biological replicates).

FIGS. 10A-10C demonstrates the outcomes of evaluating miR146a-loaded liposomes (Lip-miR-146a) using VSA high-throughput functional assays. FIG. 10A shows the quantification of atherosclerosis-related gene expression of VSAs treated with or without free miR-146a or Lip-miR-146a using qRT-PCR to show the therapeutic effect on atherosclerosis on the genetic level. Bars and error bars represent the mean±s.e.m. (n=5 biological replicates). FIG. 10B shows the quantification of cytokine secretion of VSAs treated with or without free miR-146a or Lip-miR-146a using ELISA to show the therapeutic effect on atherosclerosis-induced inflammation on the molecular level. Bars and error bars represent the mean±s.e.m. (n=5 biological replicates). FIG. 10C shows the quantification of the effect of miR-146a on foam cell formation using VSA high-throughput foam cell assay.

FIG. 11A Transmission image of hUVSMC sheet. FIG. 11B Fluorescent image of hUVSMC sheet after live and dead staining. FIG. 1C Transmission of hSVECs of the hSVEC-hUVSMC sheet. FIG. 11D Fluorescent image of DAF-stained hSVECs of the hSVEC-hUVSMC sheet.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a peptide amphiphile” includes a plurality of such peptide amphiphiles, reference to “the peptide amphiphile” is a reference to one or more peptide Amphiphiles and equivalents thereof known to those skilled in the art, and so forth.

The word “or” as used herein means any one member of a particular list and also includes any combination of members of that list.

As used herein, the term“nanomatrix vascular sheet” is a three-dimensional cell sheet with multiple layers of cells embedded in a nanomatrix hydrogel sheet. In some aspects, the vascular sheet can be an artery sheet, vein sheet, or artery sheet combined with vein sheet. For example, artery sheets can be exclusively composed of artery cells, while vein sheets are comprised solely of vein cells. In some aspects, although both arteries and veins can comprise smooth muscles cells, endothelial cells, and fibroblast cells, there are distinct attributes between arteries and veins that stem primarily from their unique structural and functional roles within the cardiovascular system. In some aspects, these differences extend to the cellular composition and arrangement within these vascular components. In some aspects, artery sheets comprise a predominantly thick artery smooth muscle cell layer, which constitutes the media layer. In contrast, vein sheets can contain fewer vein smooth muscle cells and possess a thinner media layer.

As used herein, the term “a therapeutic candidate” is any substance that has the potential to cure, mitigate or prevent atherosclerosis. These agents include but are not limited to naturally occurring substances such as herbs, plants, or minerals, synthesized chemical compounds in a laboratory to create new drugs, biological agents, lipids, proteins, peptides, antibodies, polysaccharides, nucleic acids such as microRNA, DNA, or siRNA. The test agents come in various forms, including tablets, capsules, injectables, patches, creams, gels, solutions, suspensions, and powders. They work by interacting with specific receptors, enzymes, or other molecular targets in the body to produce a desired therapeutic effect.

As used herein, the term, “peptide amphiphiles” (PAs) are a class of biomolecules consisting of a peptide sequence and a hydrophobic tail, which can self-assemble into supramolecular structures such as nanofibers, nanotubes, and micelles. The peptide sequence in PAs is typically composed of amino acids with hydrophilic properties, such as lysine or glutamic acid, while the hydrophobic tail is usually composed of a long chain of alkyl or aryl groups. As used herein, the term “PA-GTAGLIGQ-RGDS” is an example of a peptide amphiphile that consists of a hydrophobic tail and a hydrophilic head group linked by a peptide sequence containing the amino acid residues arginine (R), glycine (G), and aspartic acid (D).

As used herein, a nanomatrix is three-dimensional hydrogel network of peptide amphiphiles that are capable of absorbing and retaining large amounts of water. They mimic the properties of the extracellular matrix (ECM) and provide a suitable microenvironment for cells to proliferate, migrate, and interact with each other.

As used herein, the term “inflammation-activated nanomatrix fibroblast sheet” or “inflammatory nanomatrix fibroblast sheet” is a three-dimensional sheet with multiple layers of inflammation-activated fibroblast embedded in the nanomatrix hydrogel sheet. Inflammation-activated fibroblasts are a type of fibroblast cell that has been activated as a result of an inflammatory response. Inflammatory mediators, such as cytokines and chemokines, can activate fibroblasts and trigger changes in their morphology, function, and gene expression. In response to inflammatory stimuli, fibroblasts within the arterial wall can become activated and secrete various cytokines and growth factors, which can further promote inflammation and contribute to the formation of atherosclerotic plaques. In particular, the activation of fibroblasts in the fibrous cap of an atherosclerotic plaque can lead to the production of extracellular matrix proteins, such as collagen, that contribute to plaque stability.

As used herein, the term “inflammation-activated nanomatrix SMC sheet” or “inflammation-activated nanomatrix SMC sheet” is a three-dimensional sheet with multiple layers of inflammation-activated SMC embedded in the nanomatrix hydrogel sheet. Inflammation-activated smooth muscle cells refer to smooth muscle cells that have undergone activation as a result of an inflammatory response in the body. This activation can be triggered by the release of pro-inflammatory cytokines, chemokines, and growth factors in response to tissue damage, infection, or other stimuli. Inflammation-activated SMC may exhibit increased contractility, proliferation, migration, and extracellular matrix production. Activated SMCs in the arterial wall can produce and secrete a variety of pro-inflammatory mediators, including cytokines, chemokines, and growth factors, which can further promote inflammation and contribute to the formation of atherosclerotic plaques. In particular, activated SMCs can migrate from the media to the intima of the artery, where they can contribute to the formation of the fibrous cap that covers the atherosclerotic plaque.

As used herein, the term “dysfunctional endothelial cell” or “dysfunctional endothelium” is an endothelial cell or endothelium having impaired production and secretion of vasodilators, such as nitric oxide (NO), and enhanced production of pro-inflammatory cytokines, chemokines, and adhesion molecules in response to inflammation. In some aspects, dysfunctional endothelial cells can lead to the recruitment and activation of inflammatory cells, such as monocytes and T-cells, which can further promote inflammation and contribute to the formation of atherosclerotic plaques. In some aspects, dysfunctional endothelial cells can also promote the accumulation of lipids and cholesterol within the arterial wall, leading to the formation of foam cells, which are a hallmark of early atherosclerotic lesions.

As used herein, the term “monocyte recruitment and adhesion” refers to an orchestrated process by which these white blood cells are attracted into the arterial walls where atherosclerotic lesions are forming. In some aspect, the monocyte recruitment is initiated by various factors, including chemotactic signals released by injured or dysfunctional endothelial cells of arteries. In some aspects, monocyte recruitment is triggered by the presence of oxidized low-density lipoprotein (LDL) cholesterol particles that have been deposited within the arterial wall. These oxidized LDL particles can provoke an inflammatory response, leading to the release of chemokines and adhesion molecules. These molecules create a gradient that guides monocytes to the site of inflammation and encourages their adhesion to the endothelial cells.

As used herein, the term “monocyte differentiation into macrophages” refers to monocytes that undergo transformative changes to become specialized immune cells known as macrophages. In some aspects, monocytes differentiate into M1 macrophages (Pro-inflammatory Macrophages) under the influence of pro-inflammatory cytokines such as interferon-gamma (IFN-γ), which are characterized by their enhanced production of pro-inflammatory cytokines (e.g., TNF-alpha, IL-1beta, IL-6) and reactive oxygen species (ROS) and contribute to inflammation and the formation of atherosclerotic plaques. In some aspects, monocyte differentiation into M2 macrophages is induced by anti-inflammatory cytokines, which are involved in tissue repair, anti-inflammatory responses, and clearance of cellular debris. M2 macrophages produce factors that promote tissue healing and remodeling. In certain stages of atherosclerosis, M2 macrophages can contribute to the resolution of inflammation and tissue repair. The balance between M1 and M2 macrophages within atherosclerotic plaques can influence the plaque's stability and overall progression.

As used herein, the term “foam cell” is a macrophage cell after ingesting large amounts of LDL cholesterol particles, thus becoming foamy. In some aspect, a foam cell is a lipid-laden macrophage. In some aspects, foam cells can accumulate in the arterial wall, contributing to the progression of atherosclerosis by promoting inflammation, attracting more immune cells, and eventually leading to the formation of a fibrous cap over the plaque. In some aspect, foam cells lay the foundation for the development of fatty streaks-a crucial early stage in atherosclerosis that can be promoted into a more advanced plaque by inflammation.

As used herein, the term “calcification” refers to the process in which calcium deposits accumulate within the walls of arteries. In some aspects, calcification occurs as a response to the inflammatory processes associated with atherosclerosis, which can make the plaque more rigid and less flexible, further narrowing the artery and potentially leading to complications. Calcified plaques can be associated with an increased risk of certain cardiovascular events, such as heart attacks and strokes.

As used herein, the term “atherosclerotic plaque” refers to the initial stages of the development of an atherosclerotic lesion within the walls of arteries. These plaques can be composed of dysfunctional endothelial cells, monocytes, macrophages, foam cells, activated fibroblast and SMCs, and fatty streaks, which accumulate within the artery. In some aspects, a plaque shows an inflammatory response and secretes inflammatory cytokines. In some aspects, a plaque also shows calcification.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Nanomatrix Cell Sheets

### 1. Trilayer Nanomatrix Vascular Sheet

Disclosed are nanomatrix vascular sheets (VS) comprising a first layer, wherein the first layer is a nanomatrix sheet having fibroblasts, a second layer, wherein the second layer is a nanomatrix sheet having smooth muscle cells (SMCs), and a third layer, wherein the third layer comprises endothelial cells (ECs). Thus, in some aspects, the first two layers can be nanomatrix cell sheets and the third layer can be a layer of seeded cells. In some aspects, disclosed are nanomatrix vascular sheets comprising a Nanomatrix fibroblast sheet comprising fibroblasts and peptide amphiphiles, a Nanomatrix SMC sheet comprising SMCs and peptide amphiphiles, and seeded ECs.

In some aspects, the disclosed nanomatrix vascular sheets can comprise five layers. In some aspects, the nanomatrix vascular sheets can further comprise a layer of seeded fibroblasts and/or SMCs in between the first layer and the second layer. Thus, in some aspects, the seeded fibroblasts and/or SMCs are on the surface of the first layer (and thus could be considered to be on the surface of the second layer once the second layer has been placed on the first layer). This is different than the cells that are embedded within the nanomatrix cell sheets. In some aspects, the nanomatrix vascular sheets can further comprise a layer of seeded SMCs. Thus, in some aspects, the seeded SMCs are on the surface of the second layer. In some aspects, the surface of the second layer means the surface opposite of the surface that is facing the first layer. Thus, disclosed are nanomatrix vascular sheets comprising a first layer, wherein the first layer is a nanomatrix sheet having fibroblasts; a second layer, wherein the second layer comprises seeded fibroblasts and/or SMCs; a third layer, wherein the third layer is a nanomatrix sheet having SMCs; a fourth layer, wherein the fourth layer comprises seeded SMCs; and a fifth layer, wherein the fifth layer comprises ECs with or without a peptide amphiphile. Another way of describing the five layer Nanomatrix vascular sheet is a Nanomatrix vascular sheet comprising a Nanomatrix fibroblast sheet comprising fibroblasts and peptide amphiphiles, seeded fibroblasts and/or SMCs, a Nanomatrix SMC sheet comprising SMCs and peptide amphiphiles, seeded SMCs, and seeded ECs with or without a peptide amphiphile.

In some aspects, the nanomatrix vascular sheet is square, circular, or Y shaped. In some aspect, the nanomatrix vascular sheet can be any shape or size. In some aspects, the Nanomatrix vascular sheet is designed to fit a culture dish.

In some aspects, the trilayer nanomatrix vascular sheet mimics the layered structure of the native vascular wall composed of tunica intima, media, and adventitia.

In some aspects, the nanomatrix vascular sheet is three dimensional.

In some aspects, any of the disclosed nanomatrix cell sheets can further comprise a cross-linking agent such as, but not limited to, calcium chloride, MgCL₂, ZnCL₂, ALCL₃ or FeCL₃.

i. Nanomatrix

In some aspects, the first layer and the second layer are nanomatrix sheets comprising fibroblasts and SMCs, respectively. In some aspects, the fibroblasts of the first layer and the SMCs of the second layer are embedded within the nanomatrix cell sheet.

In some aspects, the nanomatrix is a 3D hydrogel network, for example, a peptide amphiphile-based hydrogel network. In some aspects, the nanomatrix, of both the first and second layers, is a hydrogel.

In some aspects, the nanomatrix cell sheets are square, circular, or Y shaped. Thus, in some aspects, the Nanomatrix cell sheets are the same shape as the end product, the disclosed nanomatrix vascular sheet.

In some aspects, the Nanomatrix cell sheet of the first layer and second layer comprise peptide amphiphiles.

In some aspects, cells can be encapsulated in the peptide amphiphile nanomatrix. In some aspects, this can involve using a cross-linking mist such as, but not limited to, calcium chloride, MgCL2, ZnCL2, ALCL3 or FeCL3 mist.

a. Peptide Amphiphiles

A peptide amphiphile (PA) is a molecule that possesses an amphiphilic structure typically composed of a hydrophilic peptide sequence and a hydrophobic tail. Due to the amphiphilic nature, peptide amphiphiles can self-assemble into various structures, including sheets, spheres, rods, or disks, depending on the charge and environment (pH and salt). In addition, PA is reported to self-assemble into micelles when the PA concentration is above its critical micelle concentration. Moreover, if the hydrophilic head group of the PA is bulkier than the hydrophobic tail, cylindrical micelles known as nanofibers are formed. The driven force for self-assembly is from amino acids of the PA. For instance, the negatively charged amino acids in the backbone of the PA improve the PA's solubility. However, when the negative charge of the amino acids is eliminated by lowing the pH of the PA solution or introducing the divalent ions into the PA system, PA self-assembles into specific stable structures. In addition, it should be noted that the presence of hydrogen bonds among the amino acids of the backbone of the PA leads to the formation of cylindrical structures; otherwise, spherical structures would form.

In some aspects, the nanomatrix, of both the first and second layers, comprises a peptide amphiphile comprising a hydrophobic tail and a hydrophilic peptide sequence.

There are two types of hydrophilic peptide sequences in the peptide amphiphiles that can be for making the disclosed compositions: degradation sequence, and cell-adhesive sequence.

In an aspect, the first peptide sequence of the peptide amphiphiles can be a degradation sequence, comprising an amino acid sequence that undergoes cell-mediated proteolytic degradation. The degradation sequence can comprise an MMP2 specific cleavage site, wherein comprises an amino acid sequence Gly-Thr-Ala-Gly-Leu-Ile-Gly-Gln (GTAGLIGQ; SEQ ID NO:1). MMPs are zinc-dependent endopeptidases belonging to a larger family of proteases known as the metzincin Superfamily. The MMPs can be divided into four types, the collagenases, the gelatinases, the stromelysins, and the membrane-type MMPs (MT-MMPs), dependent on the MMPs' substrate specificity and intercellular location. In particular, the collagenases are responsible for degrading triple-helical collagens, the significant components of bone and cartilage, into distinctive fragments. The traditional collagenase family includes MMP1, MMP8, MMP13, and MMP18, while MMP2 and MM9 belong to gelatinases. The primary substrates of the gelatinases are type IV collagen and gelatin. In contrast to collagenase, the stromelysins can cleave extracellular matrix proteins but not the triple-helical fibrillar collagens, including MMP3, MMP10, and MMP11. In addition to the discussed MMPs, other MMPs, such as MMP14, MMP15, MMP16, MMP17, MMP24, and MMP25, are MT-MMPs.

In an aspect, cell adhesive ligand comprises the amino acid sequence of Arg-Gly-Asp-Ser (RGDS; SEQ ID NO:2) can be used. Integrins are a family of cell adhesion receptors that play a crucial role in cell-to-cell and cell-to-extracellular matrix interactions. The RGDS sequence specifically interacts with certain integrin subtypes, such as αvβ3 and α5β1, found on various cell types. When cells encounter the RGDS sequence, it can facilitate their attachment to surfaces, which is a critical step in processes like wound healing, tissue regeneration, and the formation of new blood vessels (angiogenesis). n an aspect, cell adhesive ligand comprises the amino acid sequence of endothelial cell adhesive sequences comprise the amino acid sequence of Tyr-Ile-Gly-Ser-Arg (YIGSR; SEQ ID NO:2) can be used. YIGSR is a synthetic laminin-derived pentapeptide. Laminins are a non-collagenous glycoprotein from basement membranes, essential for building a cellular network that connects the intracellular and extracellular components. YIGSR has been shown to improve cell adhesion, regulate myoblast cell function, and promote laminin receptor binding. Incorporating the YIGSR sequence in polyurethane has been shown to enhance endothelial cell adhesion and spreading but inhibit smooth muscle cell proliferation. In some aspects, peptide amphiphiles with YIGSR sequence can significantly improved endothelial cell adhesion, spreading, and proliferation while remarkably reducing platelet adhesion. In some aspects, the cell adhesive sequence can comprise the amino acid sequence of RGDS.

In some aspects, the hydrophobic tail of the peptide amphiphile can comprise a moiety having an optionally substituted C4 or larger alkyl chain. Thus, the hydrophobic tail can comprise a moiety having an optionally substituted C6 to C28 or a larger alkyl chain. Thus, the hydrophobic tail can comprise a moiety having an optionally substituted C10 to C25 or larger alkyl chain. Thus, the hydrophobic tail can comprise a moiety having an optionally substituted C4, C5, C6, C7, C8, C9, C10, C11, C12, C13, C14, C15, C16, C17, C18, C19, C20, C21, C22, C23, C24, C25, C26, C27, C28, or larger alkyl chain. Thus, the hydrophobic tail can comprise a moiety having an optionally substituted C5 or C16 alkyl chain.

In some aspects, the peptide amphiphile is PA-GTAGLIGQ-RGDS, composed of the amino acid sequence of Arg-Gly-Asp-Ser (RGDS; SEQ ID NO:2) and matrix metalloproteinase-2 (MMP-2)-mediated degradable sites (GTAGLIGQ; SEQ ID NO:1), which is a biocompatible and biodegradable peptide amphiphile, which can be self-assembled into an extracellular matrix-mimicking nanomatrix hydrogel to provide a 3D environment for cell growth and remodeling, and used for developing 3D in vitro system. In some aspects, the peptide amphiphile is a combination of PA-GTAGLIGQ-RGDS with other types of peptide Amphiphiles. In some aspects, the peptide amphiphile is a combination of PA-GTAGLIGQ-RGDS and at least one PA-GTAGLIGQ-YIGSR. PA-GTAGLIGQ-YIGSR, is a biocompatible and biodegradable endothelial-mimicking peptide amphiphile, is composed of an endothelial adhesive ligand (YIGSR; SEQ ID NO:3), and matrix metalloproteinase-2 (MMP-2)-mediated degradable sites (GTAGLIGQ; SEQ ID NO:1). YIGSR is a synthetic laminin-derived pentapeptide. Laminins are a non-collagenous glycoprotein from basement membranes, essential for building a cellular network that connects the intracellular and extracellular components. YIGSR has been shown to improve cell adhesion, regulate myoblast cell function, and promote laminin receptor binding. Incorporating the YIGSR sequence in polyurethane has been shown to enhance endothelial cell adhesion and spreading but inhibit smooth muscle cell proliferation. In some aspects, peptide amphiphiles with YIGSR sequence can significantly improve endothelial cell adhesion, spreading, and proliferation while remarkably reducing platelet adhesion. In some aspects, the peptide amphiphile is a combination of PA-GTAGLIGQ-RGDS and at least one PA-GTAGLIGQ-K5-YIGSR or PA-K5-GTAGLIGQ-YIGSR, composed of a nitric oxide-producing sequence comprising an amino acid sequence Lys-Lys-Lys-Lys-Lys (SEQ, ID NO:4:K5), matrix metalloproteinase-2 (MMP-2)-mediated degradable sites, and endothelial adhesive ligand (YIGSR; SEQ ID NO:3). In some aspect, the peptide amphiphile is a combination of PA-GTAGLIGQ-RGDS and at least one PA-GTAGLIGQ-K5-YIGSR-NO or PA-K5-GTAGLIGQ-YIGSR-NO. In some aspect, the peptide amphiphile is a combination of PA-GTAGLIGQ-RGDS and at least one PA-GTAGLIGQ-K5-NO or PA-K5-GTAGLIGQ-NO, comprising an amino acid sequence, including Lys-Lys-Lys-Lys-Lys (SEQ, ID NO:4: K5) and matrix metalloproteinase-2 (MMP-2), and nitric oxide.

In some aspects, the peptide amphiphile is a combination of PA-GTAGLIGQ-RGDS and one or more extracellular matrix molecules. In some aspects, the one or more extracellular matrix molecules is collagen, elastin, fibronectin, hyaluronic acid, chondroitin sulfate, heparan sulfate, or proteoglycans.

In some aspects, the peptide amphiphiles in one or both of the nanomatrix cell sheets are crosslinked.

ii. Cells

In some aspects, each layer of the nanomatrix vascular sheet comprises cells. For example, in some aspects, the first layer comprises fibroblasts, the second layer comprises SMCs, and the third layer comprises endothelial cells. And, in some aspects, there can be seeded fibroblasts or SMCs between the first and second layers and there can be seeded SMCs between the second and third layers.

In some aspects, the fibroblasts can be, but are not limited to, human aortic adventitial fibroblasts, primary human coronary artery fibroblasts, primary human cardiac fibroblasts, primary human brain vascular fibroblasts, primary human carotid artery fibroblast, primary pulmonary vein fibroblast, or primary human vein fibroblasts.

In some aspects, the smooth muscle cells can be, but are not limited to, primary human umbilical artery SMCs, primary human coronary artery SMCs, primary human pulmonary artery SMCs, primary human carotid artery SMCs, primary human brain vascular SMCs, primary human vein SMCs, primary human aortic SMCs, or human stem cell-derived SMCs.

In some aspects, the endothelial cells can be, but are not limited to, human primary endothelial cells. In some aspects, the human primary endothelial cells are primary human umbilical vein ECs, primary human coronary artery ECs, primary human pulmonary artery ECs, primary human carotid artery ECs, primary human brain microvascular ECs, primary human vein ECs, primary human aortic ECs, mesenchymal stem cell-derived ECs, human induced pluripotent stem cell-derived ECs, or human embryonic stem cell-derived ECs.

In some aspects, the endothelial cells of the third layer are dysfunctional. In some aspects, the presence of dysfunctional endothelial cells makes the Nanomatrix vascular sheet a atherosclerotic nanomatrix vascular sheet. In some aspects, the cells are cultured to become dysfunctional before seeding as the third layer. In some aspects, the ECs do not become dysfunctional until after culturing with a culture medium comprising Dulbecco's Modified Eagle Medium (DMEM), TNF-α, Ox-LDL, and serum. In some aspects, when the nanomatrix vascular sheet is a atherosclerotic nanomatrix vascular sheet comprising dysfunctional ECs, the fibroblasts and the SMCs are inflammatory (or inflammation-activated) fibroblasts and inflammatory (or inflammation-activated) SMCs, respectively.

In some aspects, the concentration of TNF-α can be selected from a range of 10 ng/mL to 100 ng/mL. In some aspects, the concentration of TNF-α can be 40 ng/mL. In some aspects, the concentration of Ox-LDL can be selected from a range of 15 μg/mL to 50 μg/mL. In some aspects, the concentration of Ox-LDL can be at least 30 μg/mL. In some aspects, the concentration of serum can be selected from a range of 0.2% to 1%. In some aspects, the concentration of serum can be 0.5%. In some aspects, FBS can be used as the serum source.

iii. Nanomatrix Vascular Sheet with One or More Features of Atherosclerosis

Disclosed is a VS with one or more features of atherosclerosis, include a VS comprises dysfunctional endothelium (VS-DE), VS comprises monocyte adhesion (VS-MA), VS comprises macrophage formation (VS-MF), or VS with atherosclerosis (VS-AS). In some aspects, VS-AS is a atherosclerotic VS. In some aspects, the atherosclerosis feature includes, but not limited to, dysfunctional endothelium, monocytes, macrophages, foam cells, fatty steak, calcification, lipid, cytokines, chemokines, matrix metalloproteinases, growth factors, or reactive oxygen species

a. Nanomatrix Vascular Sheet with Dysfunctional Endothelium (VS-DE)

Disclosed is a VS-DE comprising a VS with dysfunctional endothelium.

Disclosed is a VS-DE comprising dysfunctional endothelium, inflammation-activated SMC, or inflammation-activated fibroblast layers, a key feature of atherosclerosis initiation. In some aspects, the cells are cultured to become dysfunctional before seeding as the third layer of VS to make VS-DE. In some aspects, the ECs for making VS-DE do not become dysfunctional until after culturing with a culture medium comprising Dulbecco's Modified Eagle Medium (DMEM), TNF-α, Ox-LDL, and serum. In some aspects, the concentration of TNF-α can be selected from a range of 10 ng/mL to 100 ng/mL. In some aspects, the concentration of TNF-α can be 40 ng/mL. In some aspects, the concentration of Ox-LDL can be selected from a range of 15 μg/mL to 50 μg/mL. In some aspects, the concentration of Ox-LDL can be at least 30 μg/mL. In some aspects, the concentration of serum can be selected from a range of 0.2% to 1%. In some aspects, the concentration of serum can be 0.5%. In some aspects, FBS can be used as the serum source.

Disclosed is a VS-ED comprising a first layer, wherein the first layer is a nanomatrix sheet having fibroblasts, a second layer, wherein the second layer is a nanomatrix sheet having smooth muscle cells (SMCs), and a third layer, wherein the third layer comprises endothelial cells (ECs) with or without PAs, wherein the endothelial cells are dysfunctional, wherein the fibroblasts are inflammation-activated fibroblasts, wherein the SMCs are inflammation-activated SMCs. In some aspects, VS-ED, comprise one or more additional features such as ROS, cytokines, growth factors, matrix metalloproteinases or chemokines.

Disclosed is a VS-DE comprising a first layer, wherein the first layer is a nanomatrix sheet having fibroblasts; a second layer, wherein the second layer comprises seeded fibroblasts or seeded SMCs; a third layer, wherein the third layer is a nanomatrix sheet having smooth muscle cells (SMCs), a fourth layer, wherein the fourth layer comprises seeded SMCs; and a fifth layer, wherein the fifth layer comprises endothelial cells (ECs), wherein the endothelial cells are dysfunctional, wherein the fibroblasts are inflammation-activated fibroblasts, wherein the SMCs are inflammation-activated SMCs. In some aspects, VS-ED comprise one or more additional features such as ROS, cytokines, growth factors, matrix metalloproteinases or chemokines.

b. Nanomatrix Vascular Sheet with Monocytes (VS-MO)

Disclosed is a VS-MO that is a VS-DE further comprising monocyte adhesion to the sheet.

Disclosed is a VS-MO comprising endothelial dysfunction, inflammation-activated SMC, or inflammation-activated fibroblast layers and monocyte adhesion to the sheet. In some aspects, the ECs are cultured to become dysfunctional before seeding as the third layer of VS to make VS-MO. In some aspects, the ECs for making VS-MO do not become dysfunctional until after culturing with monocytes and a culture medium comprising Dulbecco's Modified Eagle Medium (DMEM), TNF-α, Ox-LDL, and serum. In some aspects, the concentration of TNF-α can be selected from a range of 10 ng/mL to 100 ng/mL. In some aspects, the concentration of TNF-α can be 40 ng/mL. In some aspects, the concentration of Ox-LDL can be selected from a range of 15 μg/mL to 50 μg/mL. In some aspects, the concentration of Ox-LDL can be at least 30 μg/mL. In some aspects, the concentration of serum can be selected from a range of 0.2% to 1%. In some aspects, the concentration of serum can be 0.5%. In some aspects, FBS can be used as the serum source. In some aspects, the monocyte concentration can be selected from a range of 0.01 million/mL to 5 million/mL.

Disclosed is a VS-MO comprising a first layer, wherein the first layer is a nanomatrix sheet having fibroblasts, a second layer, wherein the second layer is a nanomatrix sheet having smooth muscle cells (SMCs), a third layer, wherein the third layer comprises endothelial cells (ECs), wherein the endothelial cells are dysfunctional, wherein the fibroblasts are inflammation-activated fibroblasts, wherein the SMCs are inflammation-activated SMCs, and monocytes adhesive to the sheet. In some aspects, VS-MO, comprise one or more additional features such as ROS, cytokines, growth factors, matrix metalloproteinases or chemokines.

Disclosed is a VS-MO comprising a first layer, wherein the first layer is a nanomatrix sheet having fibroblasts; a second layer, wherein the second layer comprises seeded fibroblasts or seeded SMCs; a third layer, wherein the third layer is a nanomatrix sheet having smooth muscle cells (SMCs), a fourth layer, wherein the fourth layer comprises seeded SMCs; a fifth layer, wherein the fifth layer comprises endothelial cells (ECs), wherein the endothelial cells are dysfunctional, wherein the fibroblasts are inflammation-activated fibroblasts, wherein the SMCs are inflammation-activated SMCs, and monocytes adhesive to the sheet. In some aspects, VS-MO comprise one or more additional features such as ROS, cytokines, growth factors, matrix metalloproteinases or chemokines.

In some aspects, the human monocytes can be derived from human monocyte cell lines, such as, but not limited to, THP-1, U937, MM12, MUTZ-3, or MDM2, or indirectly isolated from human blood as primary human monocytes.

c. Nanomatrix Vascular Sheet with Macrophages (VS-MA)

Disclosed is a VS-MA that is a VS-MO further comprising macrophage formation on the sheet.

Disclosed is a VS-MA comprising endothelial dysfunction, inflammation-activated SMC, inflammation-activated fibroblast layers, monocyte adhesion to the sheet, and macrophage formation on the sheet. In some aspects, the ECs are cultured to become dysfunctional before seeding as the third layer of VS to make VS-MO. In some aspects, the ECs for making VS-MO do not become dysfunctional until after culturing with a culture medium comprising Dulbecco's Modified Eagle Medium (DMEM), TNF-α, Ox-LDL, and serum. In some aspects, the concentration of TNF-α can be selected from a range of 10 ng/mL to 100 ng/mL. In some aspects, the concentration of TNF-α can be 40 ng/mL. In some aspects, the concentration of Ox-LDL can be selected from a range of 15 μg/mL to 50 μg/mL. In some aspects, the concentration of Ox-LDL can be at least 30 μg/mL. In some aspects, the concentration of serum can be selected from a range of 0.2% to 1%. In some aspects, the concentration of serum can be 0.5%. In some aspects, FBS can be used as the serum source.

Disclosed is a VS-MA comprising a first layer, wherein the first layer is a nanomatrix sheet having fibroblasts, a second layer, wherein the second layer is a nanomatrix sheet having smooth muscle cells (SMCs), a third layer, wherein the third layer comprises endothelial cells (ECs), wherein the endothelial cells are dysfunctional, wherein the fibroblasts are inflammation-activated fibroblasts, wherein the SMCs are inflammation-activated SMCs, monocytes adhesive to the sheet, and macrophage formation on the sheet. In some aspects, VS-MA, comprise one or more additional features such as ROS, cytokines, growth factors, matrix metalloproteinases, or chemokines.

Disclosed is a VS-MA comprising a first layer, wherein the first layer is a nanomatrix sheet having fibroblasts; a second layer, wherein the second layer comprises seeded fibroblasts or seeded SMCs; a third layer, wherein the third layer is a nanomatrix sheet having smooth muscle cells (SMCs), a fourth layer, wherein the fourth layer comprises seeded SMCs; a fifth layer, wherein the fifth layer comprises endothelial cells (ECs), wherein the endothelial cells are dysfunctional, wherein the fibroblasts are inflammation-activated fibroblasts, wherein the SMCs are inflammation-activated SMCs, monocytes adhesive to the sheet, and macrophage formation on the sheet. In some aspects, VS-MA comprise one or more additional features such as ROS, cytokines, growth factors, matrix metalloproteinases or chemokines.

In some aspects, the human macrophages can be derived from human monocyte cell lines or primary human monocytes. In some aspects, the macrophages can be differentiated macrophages. In some aspects, the macrophages can be either inflammatory M1 macrophages, anti-inflammatory M2 macrophages, or both.

d. Nanomatrix Vascular Sheet with Atherosclerosis (VS-AS)

Disclosed is VS-AS comprising the disclosed VS-MF and foam cells and fatty streak. In some aspects, the VS-AS further comprise calcification.

Disclosed is VS-AS having dysfunctional endothelium, monocytes, macrophages, foam cells, lipid, activated fibroblast and SMCs, and fatty streaks. In some aspect, the atherosclerotic nanomatrix vascular sheet can secret one or more key substances, such as cytokines and chemokine, matrix metalloproteinases, reactive oxygen species (ROS), and growth factors.

Disclosed is VS-AS comprising a first layer, wherein the first layer is a nanomatrix sheet having fibroblasts, a second layer, wherein the second layer is a nanomatrix sheet having smooth muscle cells (SMCs), and a third layer, wherein the third layer comprises endothelial cells (ECs), wherein the endothelial cells are dysfunctional, wherein the fibroblasts are inflammation-activated fibroblasts, wherein the SMCs are inflammation-activated SMCs, wherein the atherosclerotic nanomatrix vascular sheet comprises monocytes, macrophages, foam cells, lipid, fatty streaks, and one or more the key substances, such as cytokines, chemokines, matrix metalloproteinases, growth factors, and reactive oxygen species. In some aspects, the atherosclerotic vascular sheet further comprises calcification.

Disclosed are atherosclerotic nanomatrix vascular sheets comprising a first layer, wherein the first layer is a nanomatrix sheet having fibroblasts; a second layer, wherein the second layer comprises seeded fibroblasts or seeded SMCs; a third layer, wherein the third layer is a nanomatrix sheet having smooth muscle cells (SMCs), a fourth layer, wherein the fourth layer comprises seeded SMCs; and a fifth layer, wherein the fifth layer comprises endothelial cells (ECs), wherein the endothelial cells are dysfunctional, wherein the fibroblasts are inflammation-activated fibroblasts, wherein the SMCs are inflammation-activated SMCs, wherein comprises monocytes, macrophages, foam cells, lipid, fatty streaks, and one or more the key substances, such as cytokines, chemokines, matrix metalloproteinases, growth factors and reactive oxygen species. In some aspect, the atherosclerotic vascular sheet further comprises calcification.

In some aspects, the human monocytes of the disclosed VS-AS can be derived from human monocyte cell lines, such as, but not limited to, THP-1, U937, MM12, MUTZ-3, or MDM2, or indirectly isolated from human blood as primary human monocytes. Similarly, in some aspects, the human macrophages can be derived from human monocyte cell lines or primary human monocytes. In some aspects, the macrophages can be differentiated macrophages. In some aspects, the foam cells of the disclosed VS-AS can be lipid-laden macrophages or can originate from either inflammatory M1 macrophages or anti-inflammatory M2 macrophages. In some aspects, the lipids of the disclosed VS-AS can be low density lipoproteins, oxidized low density lipoproteins, or both. In some aspects, the cytokines can include, but are not limited to, inflammatory cytokines such as tumor necrosis factor-alpha (TNF-alpha), interleukin-1 beta (IL-1beta), interleukin-6 (IL-6), interleukin-8 (IL-8), interferon-gamma (IFN-gamma), interleukin-1alpha (IL-1α), or anti-inflammatory cytokines such as interleukin-10 (IL-10), interleukin-4 (IL-4), or interleukin-13 (IL-13). In some aspect, the chemokines, can be, but limited to, monocyte chemoattractant Protein-1 (MCP-1) or Macrophage Inflammatory Protein-Ia.

In some aspects, these components are key atherosclerotic features. Thus, the presence of these features can be indicative of atherosclerotic plaque in the nanomatrix vascular sheet.

In some aspects, a nanomatrix vascular sheet with atherosclerosis, which is a three-dimensional tissue sheet comprising multiple layers of inflammation activated nanomatrix vascular sheets and an atherosclerotic plaque, created in vitro.

In some aspects, the cytokines can be, but are not limited to, tumor necrosis factor-alpha (TNF-alpha), interleukin-1 beta (IL-1beta), interleukin-6 (IL-6), interleukin-8 (IL-8), interferon-gamma (IFN-gamma), interleukin-1alpha (IL-1α), or anti-inflammatory cytokines such as interleukin-10 (IL-10), interleukin-4 (IL-4), interleukin-13 (IL-13), or a combination thereof.

In some aspects, the macrophages are differentiated macrophages.

In some aspects, the atherosclerotic nanomatrix vascular sheet comprises an inflammation-activated nanomatrix vascular sheet and a plaque. In some aspects, an inflammation-activated nanomatrix vascular sheet refers to the trilayer Nanomatrix vascular sheet comprising an inflammation-activated nanomatrix fibroblast sheet, a layer of inflammation-activated fibroblast, an inflammation-activated nanomatrix SMC sheet, a layer of inflammation-activated SMCs, and a layer of dysfunctional ECs. In some aspects, the inflammation-activated nanomatrix fibroblast sheet comprises nanomatrix and inflammation-activated fibroblasts, wherein the inflammation-activated nanomatrix SMC sheet comprises nanomatrix and inflammation-activated SMCs. In some aspects, the nanomatrix fibroblast sheet and the nanomatrix SMC sheet become inflammation-activated nanomatrix fibroblast sheet and inflammation-activated nanomatrix SMC sheets once inducing atherosclerosis in the Nanomatrix vascular sheets. In some aspects, a plaque comprises one or more components, wherein the one or more components can be, but are not limited to, human monocyte, human macrophage, human EC, human fibroblast, human SMC, human foam cell, human low-density lipoproteins, fatty streak, cytokine, chemokine, matrix metalloproteinases, growth factors, reactive oxygen species, and/or calcification.

In some aspects, a plaque refers to the presence of components such as human monocytes, human macrophages, human ECs, human fibroblasts, human SMCs, human foam cells, human low-density lipoproteins, matrix metalloproteinases, growth factors, cytokines, chemokines, reactive oxygen species, fatty streak or calcification.

In some aspects, the fibroblasts and the SMCs in the atherosclerotic nanomatrix vascular sheet are inflammation-activated fibroblasts and inflammation-activated SMCs

### 2. Single-Layer Nanomatrix Cell Sheet

Each of the single-layer cell sheets used to make the disclosed nanomatrix vascular sheets are described herein.

In some aspects, the nanomatrix fibroblast sheet and/or Nanomatrix SMC sheet is square, circular, or Y shaped. In some aspect, the nanomatrix cell sheets can be any shape or size. In some aspects, the nanomatrix cell sheets are designed to fit a culture dish.

In some aspects, any of the disclosed nanomatrix cell sheets can further comprise a cross-linking agent such as, but not limited to, calcium chloride, MgCL₂, ZnCL₂, ALCL₃ or FeCL₃.

i. Nanomatrix Fibroblast Sheet

Disclosed are nanomatrix cell sheets comprising peptide amphiphiles and fibroblasts. In some aspects, this can be referred to as a nanomatrix fibroblast sheet or fibroblast nanomatrix sheet. In some aspects, a nanomatrix fibroblast sheet is defined as the presence of fibroblasts embedded in a nanomatrix hydrogel network. In some aspects, the nanomatrix fibroblast sheet is three-dimensional.

In some aspects, an example of the single-layer nanomatrix fibroblast sheet is pictured in FIG. TA (the top right image).

In some aspects, the fibroblasts can be, but are not limited to, human aortic adventitial fibroblasts, primary human coronary artery fibroblasts, primary human cardiac fibroblasts, primary human brain vascular fibroblasts, primary human carotid artery fibroblast, primary pulmonary vein fibroblast, or primary human vein fibroblasts.

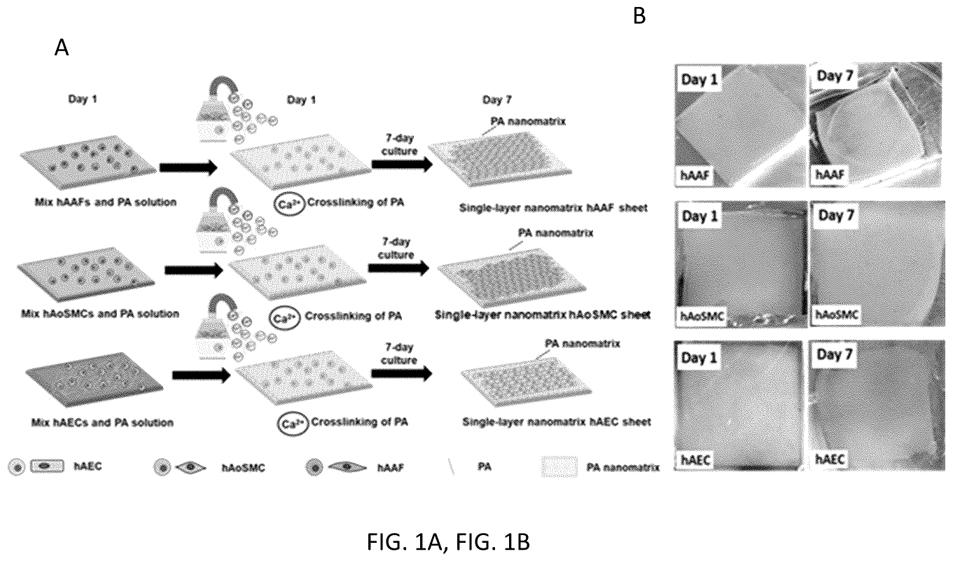

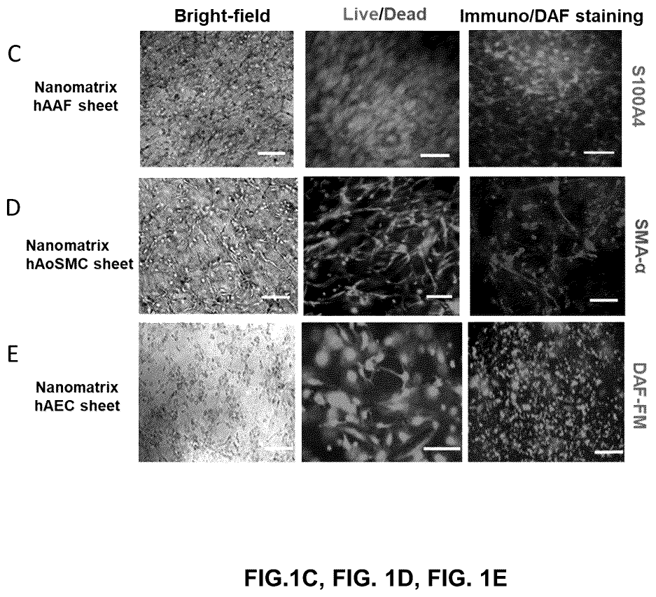

In some aspects, the Nanomatrix fibroblast sheet can further comprise a layer of seeded fibroblasts on its surface. In some aspects, these seeded fibroblasts are different from the fibroblasts that are embedded in the Nanomatrix because these fibroblasts remain on the surface of the nanomatrix cell sheet. In some aspects, the seeded fibroblasts are only present on one surface of the Nanomatrix fibroblast sheet. In some aspects, the seeded fibroblasts are present as a single layer on the surface of the Nanomatrix fibroblast sheet. In some aspects, an example of the single-layer Nanomatrix fibroblast sheet with a layer of seeded fibroblasts is pictured in FIG. 1A (middle, far right image).

ii. Nanomatrix Smooth Muscle Cell Sheet

Disclosed are nanomatrix cell sheets comprising peptide amphiphiles and SMCs. In some aspects, this can be referred to as a SMC nanomatrix sheet or nanomatrix SMC sheet. In some aspects, a nanomatrix SMC sheet is defined as the presence of SMCs embedded in a nanomatrix hydrogel network. In some aspects, the Nanomatrix SMC sheet is three-dimensional.

In some aspects, the smooth muscle cells can be, but are not limited to, primary human umbilical artery SMCs, primary human coronary artery SMCs, primary human pulmonary artery SMCs, primary human carotid artery SMCs, primary human brain vascular SMCs, primary human vein SMCs, primary human aortic SMCs, human stem cell-derived SMCs or a combination thereof.

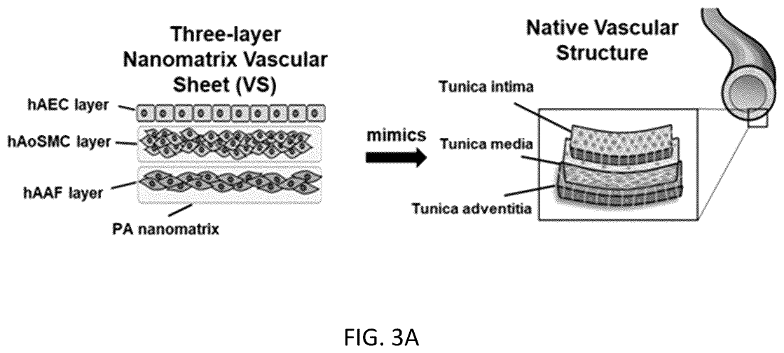

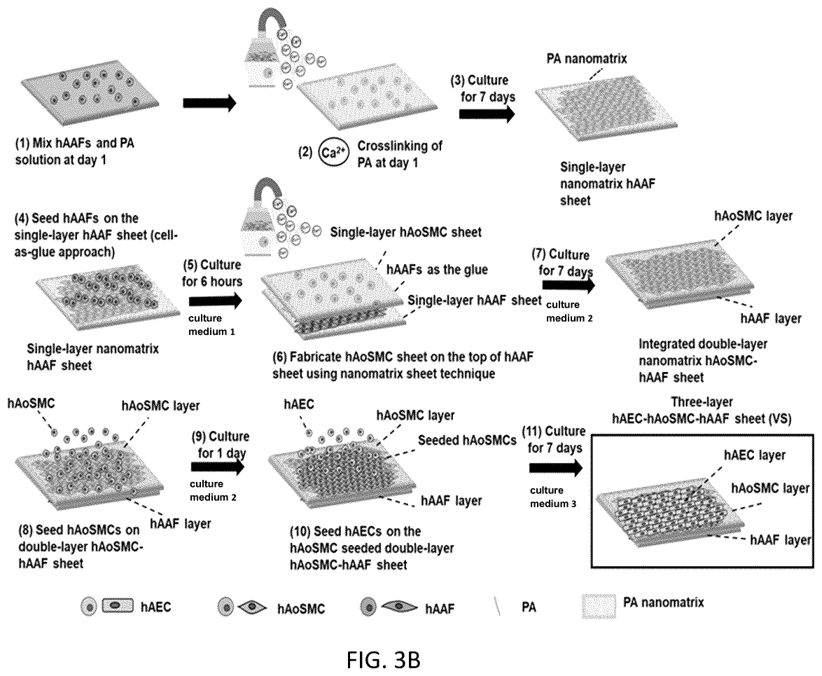

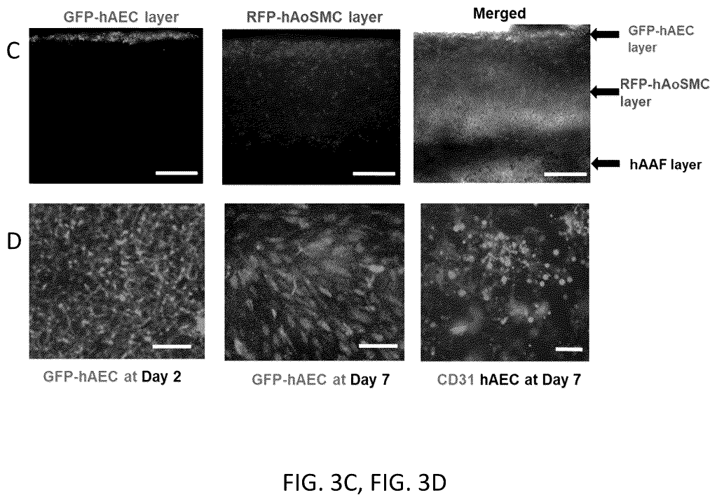

In some aspects, an example of the single-layer Nanomatrix SMC sheet is pictured in FIG. 3B (the top sheet in the middle image of the middle row).

In some aspects, the nanomatrix SMC sheet can further comprise a layer of seeded SMCs on its surface. In some aspects, these seeded SMCs are different from the SMCs that are embedded in the nanomatrix because these SMCs remain on the surface of the nanomatrix cell sheet. In some aspects, the seeded SMCs are only present on one surface of the Nanomatrix SMC sheet. In some aspects, the seeded SMCs are present as a single layer on the surface of the Nanomatrix SMC sheet.

In some aspects, an example of the single-layer Nanomatrix fibroblast sheet with a layer of seeded fibroblasts is pictured in FIG. 3B (the bottom sheet in the middle row, middle image).

iii. Nanomatrix for Both Nanomatrix Fibroblast Sheet and the Nanomatrix SMC Sheet

In some aspects, the nanomatrix for both the nanomatrix fibroblast sheet and the nanomatrix SMC sheet can be of similar components (except with different cell types).

In some aspects, the Nanomatrix fibroblast sheet and/or the Nanomatrix SMC sheet comprise cells embedded in the Nanomatrix (i.e. fibroblasts and SMCs, respectively).

In some aspects, the nanomatrix is a 3D hydrogel network, for example, a peptide amphiphile-based hydrogel network. In some aspects, the nanomatrix, of both the first and second layers, is a hydrogel.

In some aspects, the nanomatrix fibroblast sheet and the nanomatrix SMC sheet comprise peptide amphiphiles.

a. Peptide Amphiphiles

The peptide Amphiphiles are as described in the trilayer.

In some aspects, the nanomatrix fibroblast sheet and the nanomatrix SMC sheet comprise a peptide amphiphile comprising a hydrophobic tail and a hydrophilic peptide sequence.

In some aspects, the peptide amphiphile is PA-GTAGLIGQ-RGDS or a combination of PA-GTAGLIGQ-RGDS with other types of peptide amphiphiles. In some aspects, the peptide amphiphile is a combination of PA-GTAGLIGQ-RGDS and at least one PA selected from the group of PA-GTAGLIGQ-YIGSR, PA-KKKKK-GTAGLIGQ-YIGSR, PA-GTAGLIGQ-YIGSR-NO, PA-KKKKK-GTAGLIGQ-YIGSR-NO, PA-KKKKK-GTAGLIGQ, and PA-KKKKK-GTAGLIGQ-NO.

In some aspects, the peptide amphiphile is a combination of PA-GTAGLIGQ-RGDS and one or more extracellular matrix molecules. In some aspects, the one or more extracellular matrix molecules is collagen, elastin, fibronectin, hyaluronic acid, chondroitin sulfate, heparan sulfate, or proteoglycans.

In some aspects, the peptide amphiphiles in one or both of the nanomatrix fibroblast sheet and the nanomatrix SMC sheet are crosslinked.

iv. Single-Layer Nanomatrix Cell Sheet of One or More Atherosclerosis Features

Disclosed are single-layer nanomatrix cell sheet of one or more atherosclerosis features. In some aspects, the atherosclerosis feature includes, but not limited to, dysfunctional endothelium, monocytes, macrophages, foam cells, fatty steak, calcification, lipid, cytokines, chemokines, matrix metalloproteinases, growth factors, or reactive oxygen species.

a. Single-Layer Nanomatrix Cell Sheet with Monocytes (SL-MO)

Disclosed is SL-MO comprising inflammation activated single-layer nanomatrix cell sheet and monocytes, wherein the single-layer nanomatrix cell sheet is the disclosed nanomatrix fibroblast sheet or nanomatrix SMC sheet

Disclosed is SL-MO comprising inflammation-activated nanomatrix cell sheet and monocyte adhered to the sheet, wherein the inflammation-activated nanomatrix cell sheet is inflammation-activated fibroblast sheet or inflammation-activated SMC sheet. In some aspect, the single-layer nanomatrix cell sheet further comprise one or more additional features such as ROS, cytokines, growth factors, matrix metalloproteinases, or chemokines.

Disclosed is SL-MO comprising a first layer, wherein the first layer comprise an inflammation activated nanomatrix cell sheet, wherein the inflammation-activated nanomatrix cell sheet is inflammation-activated fibroblast sheet or inflammation-activated SMC sheet; a second layer, wherein the second layer comprises seeded fibroblasts, seeded SMCs, or seeded ECs; and monocyte adhesion to the sheet. In some aspect, the single-layer nanomatrix cell sheet further comprise one or more additional features such as ROS, cytokines, growth factors, matrix metalloproteinases, or chemokines.

In some aspects, the fibroblasts can be, but are not limited to, human aortic adventitial fibroblasts, primary human coronary artery fibroblasts, primary human cardiac fibroblasts, primary human brain vascular fibroblasts, primary human carotid artery fibroblast, primary pulmonary vein fibroblast, or primary human vein fibroblasts.

In some aspects, the smooth muscle cells can be, but are not limited to, primary human umbilical artery SMCs, primary human coronary artery SMCs, primary human pulmonary artery SMCs, primary human carotid artery SMCs, primary human brain vascular SMCs, primary human vein SMCs, primary human aortic SMCs, human stem cell-derived SMCs or a combination thereof. In some aspects, the fibroblasts can be, but are not limited to, human aortic adventitial fibroblasts, primary human coronary artery fibroblasts, primary human cardiac fibroblasts, primary human brain vascular fibroblasts, primary human carotid artery fibroblast, primary pulmonary vein fibroblast, or primary human vein fibroblasts.

In some aspects, the human monocytes can be derived from human monocyte cell lines, such as, but not limited to, THP-1, U937, MM12, MUTZ-3, or MDM2, or indirectly isolated from human blood as primary human monocytes.

b. Single-layer Nanomatrix Cell Sheet with Macrophages (SL-MA)

Disclosed is SL-MA comprising SL-MO and macrophages.

Disclosed is SL-MA comprising inflammation-activated nanomatrix cell sheet, monocyte adhered to the sheet, and macrophages, wherein the inflammation-activated nanomatrix cell sheet is inflammation-activated fibroblast sheet or inflammation-activated SMC sheet. In some aspect, the SL-MA further comprise one or more additional features such as ROS, cytokines, growth factors, matrix metalloproteinases, or chemokines. In some aspects, the macrophages can be derived from human monocyte cell lines or primary human monocytes. In some aspects, the macrophages can be differentiated human macrophages. In some aspects, the macrophages can be either inflammatory human M1 macrophages, anti-inflammatory human M2 macrophages, or both.

Disclosed is SL-MA comprising a first layer, wherein the first layer comprise an inflammation activated nanomatrix cell sheet, wherein the inflammation-activated nanomatrix cell sheet is inflammation-activated fibroblast sheet or inflammation-activated SMC sheet; a second layer, wherein the second layer comprises seeded fibroblasts, seeded SMCs, or seeded ECs; monocyte adhesion to the sheet, and macrophages. In some aspect, the SL-MA further comprise one or more additional features such as ROS, cytokines, growth factors, matrix metalloproteinases, or chemokines. In some aspects, the macrophages can be derived from human monocyte cell lines or primary human monocytes. In some aspects, the macrophages can be differentiated human macrophages. In some aspects, the macrophages can be either inflammatory human M1 macrophages, anti-inflammatory human M2 macrophages, or both.

c. Single-Layer Nanomatrix Cell Sheet with Foam Cell and Fatty Streak (SL-FC)

Disclosed is SL-FC comprising SL-MA, foam cell, and fatty streak, wherein foam cell is a lipid-laden macrophage, wherein the fatty streak is aggregation of foam cells. In some respects, the foam cells can be derived from either inflammatory human M1 macrophages, anti-inflammatory human M2 macrophages, or both.

### 3. Double-Layer

In some aspects, the single-layer sheets described herein can be combined (e.g. stacked on top of each other) to form a double-layer nanomatrix cell sheet.

Disclosed are double-layer nanomatrix cell sheets comprising a nanomatrix fibroblast sheet comprising peptide amphiphiles and fibroblasts; and a nanomatrix SMC sheet comprising peptide amphiphiles and smooth muscle cells.

In some aspects, there is a layer of fibroblasts between the nanomatrix fibroblast sheet and nanomatrix SMC sheet. Thus, disclosed are double-layer nanomatrix cell sheets comprising a nanomatrix fibroblast sheet comprising peptide amphiphiles and fibroblasts; a nanomatrix SMC sheet comprising peptide amphiphiles and smooth muscle cells; and a layer of fibroblasts between the nanomatrix fibroblast sheet and nanomatrix SMC sheet. In some aspects, the layer of fibroblasts between the Nanomatrix fibroblast sheet and nanomatrix SMC sheet are different than the fibroblasts in the nanomatrix fibroblast sheet because these fibroblasts are not embedded in a nanomatrix but rather are on the surface of the nanomatrix cell sheets. In some aspects, the fibroblasts in the layer of fibroblasts can be the same type of fibroblast that is present in the nanomatrix fibroblast sheet or can be a different fibroblast cell type.

In some aspects, the nanomatrix fibroblast sheet and the nanomatrix SMC sheet can be any of those as described herein.

In some aspects, the fibroblasts, both in the nanomatrix and in the fibroblast layer between the nanomatrices, can be, but are not limited to, human aortic adventitial fibroblasts, primary human coronary artery fibroblasts, primary human cardiac fibroblasts, primary human brain vascular fibroblasts, primary human carotid artery fibroblast, primary pulmonary vein fibroblast, primary human vein fibroblasts, or a combination thereof.

In some aspects, the smooth muscle cells can be, but are not limited to, primary human umbilical artery SMCs, primary human coronary artery SMCs, primary human pulmonary artery SMCs, primary human carotid artery SMCs, primary human brain vascular SMCs, primary human vein SMCs, primary human aortic SMCs, human stem cell-derived SMCs or a combination thereof.

In some aspects, an example of the double-layer nanomatrix is pictured in FIG. 3B (middle row, middle and far right images).

In some aspects, the double-layer nanomatrix can further comprise a layer of seeded SMCs on its surface. In some aspects, these seeded SMCs are different from the SMCs that are embedded in the Nanomatrix SMC sheet because these SMCs remain on the surface of the Nanomatrix cell sheet, specifically on the surface of the nanomatrix SMC sheet. In some aspects, the seeded SMCs are present as a single layer on the surface of the nanomatrix SMC sheet. In some aspects, the seeded SMCs are the same SMC type present in the nanomatrix SMC sheet. In some aspects, the seeded SMCs are a different SMC type than what is present in the nanomatrix SMC sheet.

In some aspects, an example of the double-layer nanomatrix cell sheet with a layer of seeded SMCs is pictured in FIG. 3B (bottom row, far left image).

In some aspects, the double-layer nanomatrix can further comprise a layer of endothelial cells. In some aspects, the addition of the layer of endothelial cells converts the double-layer Nanomatrix cell sheet to the trilayer Nanomatrix cell sheet described herein. In some aspects, the endothelial cells are seeded on the surface of the nanomatrix SMC sheet of the double-layer nanomatrix cell sheet. In some aspects, the endothelial cells are seeded on top of the SMCs that are seeded on the surface of the Nanomatrix SMC sheet of the double-layer nanomatrix cell sheet.

In some aspects, an example of the double-layer Nanomatrix cell sheet with a layer of seeded endothelial cells on top of the seeded SMCs is pictured in FIG. 3B (bottom row, middle and far right images).

In some aspects, the double-layer nanomatrix cell sheet is square, circular, or Y shaped. In some aspect, the double-layer nanomatrix cell sheet can be any shape or size. In some aspects, the double-layer nanomatrix cell sheet is designed to fit a culture dish.

In some aspects, any of the disclosed nanomatrix cell sheets can further comprise a cross-linking agent such as, but not limited to, calcium chloride, MgCL₂, ZnCL₂, ALCL₃ or FeCL₃.

i. Nanomatrix for Both Nanomatrix Fibroblast Sheet and the Nanomatrix SMC Sheet

In some aspects, the nanomatrix of the double-layer Nanomatrix cell sheet comprises the same components as described herein.

In some aspects, the nanomatrix for both the nanomatrix fibroblast sheet and the nanomatrix SMC sheet, of the double-layer Nanomatrix cell sheet, can be of similar components (except with different cell types).

In some aspects, the nanomatrix fibroblast sheet and the nanomatrix SMC sheet comprise cells embedded in the nanomatrix (i.e. fibroblasts and SMCs, respectively).

In some aspects, the nanomatrix is a 3D hydrogel network, for example, a peptide amphiphile-based hydrogel network. In some aspects, the nanomatrix, of both the first and second nanomatrix cell sheets, is a hydrogel.

In some aspects, the nanomatrix fibroblast sheet and the nanomatrix SMC sheet comprise peptide amphiphiles.

a. Peptide Amphiphiles

The peptide Amphiphiles are as described in the trilayer.

In some aspects, the nanomatrix fibroblast sheet and the nanomatrix SMC sheet comprise a peptide amphiphile comprising a hydrophobic tail and a hydrophilic peptide sequence.

In some aspects, the peptide amphiphile is PA-GTAGLIGQ-RGDS or a combination of PA-GTAGLIGQ-RGDS with other types of peptide amphiphiles. In some aspects, the peptide amphiphile is a combination of PA-GTAGLIGQ-RGDS and at least one PA selected from the group of PA-GTAGLIGQ-YIGSR, PA-KKKKK-GTAGLIGQ-YIGSR, PA-GTAGLIGQ-YIGSR-NO, PA-KKKKK-GTAGLIGQ-YIGSR-NO, PA-KKKKK-GTAGLIGQ, and PA-KKKKK-GTAGLIGQ-NO. In some aspects, the peptide amphiphile is a combination of PA-GTAGLIGQ-RGDS and one or more extracellular matrix molecules. In some aspects, the one or more extracellular matrix molecules is collagen, elastin, fibronectin, hyaluronic acid, chondroitin sulfate, heparan sulfate, or proteoglycans.

In some aspects, the peptide amphiphiles in one or both of the nanomatrix fibroblast sheet and the nanomatrix SMC sheet are crosslinked.

ii. Double-Layer Nanomatrix Cell Sheet with One or More Atherosclerosis Features

Disclosed are double-layer nanomatrix cell sheet of one or more atherosclerosis features. In some aspects, the atherosclerosis feature includes, but not limited to, dysfunctional endothelium, monocytes, macrophages, foam cells, fatty steak, calcification, lipid, cytokines, chemokines, matrix metalloproteinases, growth factors, or reactive oxygen species

a. Double-Layer Nanomatrix Cell Sheet with Monocytes (DL-MO)

Disclosed is a DL-MO comprising a first layer, wherein the first layer is a nanomatrix sheet having fibroblasts, a second layer, wherein the second layer is a nanomatrix sheet having smooth muscle cells (SMCs), wherein the fibroblasts are inflammation-activated fibroblasts, wherein the SMCs are inflammation-activated SMCs; and monocytes. In some aspects, the double-layer nanomatrix cell sheet with monocyte adhesion further comprise one or more additional features such as ROS, cytokines, growth factors, matrix metalloproteinases, or chemokines.

Disclosed are DL-MO comprising a first layer, wherein the first layer is a nanomatrix sheet having fibroblasts; a second layer, wherein the second layer comprises seeded fibroblasts or seeded SMCs; a third layer, wherein the third layer is a nanomatrix sheet having SMCs, a fourth layer, wherein the fourth layer comprises seeded SMCs, wherein the fibroblasts are inflammation-activated fibroblasts, wherein the SMCs are inflammation-activated SMCs; and monocytes. In some respects, the double-layer nanomatrix cell sheet with monocytes further comprises one or more additional features such as ROS, cytokines, growth factors, matrix metalloproteinases, or chemokines.

b. Double-Layer Nanomatrix Cell Sheet with Macrophages (DL-MA)

Disclosed is a DL-MA comprising DL-MO and macrophages.

Disclosed is a DL-MA comprising a first layer, wherein the first layer is a nanomatrix sheet having fibroblasts, a second layer, wherein the second layer is a nanomatrix sheet having smooth muscle cells (SMCs), wherein the fibroblasts are inflammation-activated fibroblasts, wherein the SMCs are inflammation-activated SMCs, monocytes, and macrophages. In some aspects, DL-MA, comprise one or more additional features such as ROS, cytokines, growth factors, matrix metalloproteinases, or chemokines.

Disclosed is a DL-MA comprising a first layer, wherein the first layer is a nanomatrix sheet having fibroblasts; a second layer, wherein the second layer comprises seeded fibroblasts or seeded SMCs; a third layer, wherein the third layer is a nanomatrix sheet having smooth muscle cells (SMCs); wherein the fibroblasts are inflammation-activated fibroblasts, wherein the SMCs are inflammation-activated SMCs, monocytes adhesive to the sheet, and macrophage formation on the sheet. In some aspects, VS-MA comprise one or more additional features such as ROS, cytokines, growth factors, matrix metalloproteinases or chemokines.

Disclosed is a DL-MA comprising a first layer, wherein the first layer is a nanomatrix sheet having fibroblasts; a second layer, wherein the second layer comprises seeded fibroblasts or seeded SMCs; a third layer, wherein the third layer is a nanomatrix sheet having smooth muscle cells (SMCs), a fourth layer, wherein the fourth layer comprises seeded SMCs; wherein the fibroblasts are inflammation-activated fibroblasts, wherein the SMCs are inflammation-activated SMCs; monocytes adhesive to the sheet, and macrophage formation on the sheet. In some aspects, VS-MA comprise one or more additional features such as ROS, cytokines, growth factors, matrix metalloproteinases or chemokines.

In some aspects, the macrophages of the disclosed DL-MA can be derived from human monocyte cell lines or primary human monocytes. In some aspects, the macrophages of the disclosed DL-MA can be differentiated macrophages. In some aspects, the macrophages of the disclosed DL-MA can be either inflammatory M1 macrophages, anti-inflammatory M2 macrophages, or both.

c. Double-Layer Nanomatrix Cell Sheet with Foam Cells and Fatty Streak (DL-FC)

Disclosed is a DL-FC comprising DL-MA, foam cells and fatty streak.

Disclosed is a DL-FC comprising a first layer, wherein the first layer is a nanomatrix sheet having fibroblasts, a second layer, wherein the second layer is a nanomatrix sheet having smooth muscle cells (SMCs), wherein the fibroblasts are inflammation-activated fibroblasts, wherein the SMCs are inflammation-activated SMCs; monocytes, macrophages, foam cells, and fatty streak. In some aspects, DL-FC, comprise one or more additional features such as ROS, cytokines, growth factors, matrix metalloproteinases, or chemokines.

Disclosed is a DL-FC comprising a first layer, wherein the first layer is a nanomatrix sheet having fibroblasts; a second layer, wherein the second layer comprises seeded fibroblasts or seeded SMCs; a third layer, wherein the third layer is a nanomatrix sheet having smooth muscle cells (SMCs), a fourth layer, wherein the fourth layer comprises seeded SMCs; wherein the fibroblasts are inflammation-activated fibroblasts, wherein the SMCs are inflammation-activated SMCs, monocytes adhesive to the sheet, and macrophage formation on the sheet. In some aspects, DL-FC comprise one or more additional features such as ROS, cytokines, growth factors, matrix metalloproteinases or chemokines.

In some aspects, the macrophages of DL-FC can be derived from human monocyte cell lines or primary human monocytes. In some aspects, the macrophages of DL-MA can be differentiated macrophages. In some aspects, the macrophages of DL-MA can be either inflammatory M1 macrophages, anti-inflammatory M2 macrophages, or both. In some aspects, the foam cell is a lipid-laden macrophages and the fatty streak is aggregation of foam cells. In some respects, the foam cells can be derived from either inflammatory human M1 macrophages, anti-inflammatory human M2 macrophages, or both.

### C. Methods

### 1. Methods of Producing

Disclosed are methods of producing one or more of the disclosed Nanomatrix cell sheets.

i. Producing Single-Layer Nanomatrix Cell Sheets

Disclosed are methods of producing a single-layer nanomatrix fibroblast sheet. In some aspects, are methods of producing a nanomatrix fibroblast sheet comprise combining fibroblast cells with a peptide amphiphile solution, applying a layer of the peptide amphiphile solution containing fibroblast cells over or in a substrate (e.g. multi-wells, 3D Printed PDMS chamber, glass chamber), crosslinking the peptide amphiphiles to form nanomatrix, culturing the nanomatrix sheet with fibroblast in the substrate in fibroblast complete expansion medium with 20% FBS, 1% penicillin-streptomycin and 150 ug/mL ascorbic acid to allow for cell spreading and remodeling, thereby forming a nanomatrix fibroblast sheet. In some aspects, the substrate can be, but not limited to, a well of 48 well, 96 well, or 386 well plates. The substrate can be any size or shape. In some aspect, the fibroblast density for making nanomatrix fibroblast sheet is in a range between 0.05 million cells/cm² to 5 million cells/cm². In some aspects, the culture time for nanomatrix with fibroblast cells to form nanomatrix fibroblast sheet is in the range of 1-30 days.

Disclosed are methods of producing a nanomatrix SMC sheet. In some aspects, disclosed are methods of producing a nanomatrix SMC sheet comprise combining SMCs with a peptide amphiphile solution, applying a layer of peptide amphiphile solution containing SMCs over a substrate, crosslinking the peptide amphiphiles to form an nanomatrix, culturing the nanomatrix with SMCs in SMC complete expansion medium with 20% FBS, 1% penicillin-streptomycin, and 150 μg/mL ascorbic acid to form a nanomatrix SMC sheet. In some aspects, the culture time for nanomatrix with SMCs is in the range of 1-30 days. In some aspects, the substrate can be, but not limited to, a well of 48 well, 96 well, or 386 well plates. In some aspect, the substrate can be any size or shape. In some aspect, the SMC density is in a range between 0.05 million cells/cm² to 5 million cells/cm².

In some aspects, the nanomatrix fibroblast sheet and/or the nanomatrix SMC sheet can be cultured in a humidified tissue culture incubator maintained at 37° C., 5% CO₂, and 20% O₂.

In some aspects, the crosslinking of the peptide amphiphiles in producing the nanomatrix fibroblast sheet and/or nanomatrix SMC sheet is performed in the presence of calcium chloride. In some aspects, crosslinking occurs by misting the peptide amphiphiles with calcium chloride. In some aspects, the cross-linking agent can be, but is not limited to CaCl₂, MgCL₂, ZnCL₂, ALCL₃ and FeCL₃. In some aspects, the CaCl₂ can be in the form of a mist or a solution. In some aspects, the concentration of CaCl₂ can range from 0.01M to 0.1M. In some aspects, a humidifier can be used to generate a CaCl₂ mist, which is then applied to the cells along with the peptide amphiphile solution. In some aspects, a spray bottle can be used to apply a CaCl₂ solution to the cells with the peptide amphiphile solution.

In some aspects, the fibroblasts can be, but are not limited to, human aortic adventitial fibroblasts, primary human coronary artery fibroblasts, primary human cardiac fibroblasts, primary human brain vascular fibroblasts, primary human carotid artery fibroblast, primary pulmonary vein fibroblast, or primary human vein fibroblasts.

In some aspects, the smooth muscle cells can be, but are not limited to, primary human umbilical artery SMCs, primary human coronary artery SMCs, primary human pulmonary artery SMCs, primary human carotid artery SMCs, primary human brain vascular SMCs, primary human vein SMCs, primary human aortic SMCs, or human stem cell-derived SMCs.

In some aspects, the peptide amphiphile solution comprises one or more of the peptide amphiphiles described herein and water. In some aspects, the peptide amphiphile solution comprises peptide amphiphiles comprising a hydrophobic tail and a hydrophilic peptide sequence. For example, In some aspects, the peptide amphiphile is PA-GTAGLIGQ-RGDS or a combination of PA-GTAGLIGQ-RGDS with other types of peptide amphiphiles. In some aspects, the peptide amphiphile is a combination of PA-GTAGLIGQ-RGDS and at least one PA selected from the group of PA-GTAGLIGQ-YIGSR, PA-KKKKK-GTAGLIGQ-YIGSR, PA-KKKKK-GTAGLIGQ-YIGSR-NO, PA-KKKKK-GTAGLIGQ, and PA-KKKKK-GTAGLIGQ-NO. In some aspects, the peptide amphiphile solution can further comprise one or more proteins or biomolecules typically found in the extracellular matrix, including but not limited to, collagen, elastin, fibronectin, hyaluronic acid, chondroitin sulfate, heparan sulfate, and proteoglycans.

ii. Producing Double-Layer Nanomatrix Cell Sheet

Disclosed are methods of producing a double-layer nanomatrix cell sheet. In some aspects, in order to produce a double-layer nanomatrix cell sheet, each single-layer Nanomatrix fibroblast sheet and Nanomatrix SMC sheet are first produced and then the remaining steps for producing a double-layer nanomatrix cell sheet can be performed. In some aspects, methods of producing a double-layer nanomatrix cell sheet comprise producing or forming a nanomatrix fibroblast sheet and then producing the nanomatrix SMC sheet on top of the nanomatrix fibroblast sheet.

a. Starting with Pre-Formed Nanomatrix Fibroblast Sheet and/or Nanomatrix SMC Sheet

In some aspects, the single-layer nanomatrix fibroblast sheet and/or nanomatrix SMC sheet are pre-formed and therefore, producing the double-layer nanomatrix cell sheet can begin with seeding fibroblasts on the pre-formed nanomatrix fibroblast sheet and using them as a glue to hold the nanomatrix fibroblast sheet and nanomatrix SMC sheet together.

In some aspects, both the nanomatrix fibroblast sheet and nanomatrix SMC sheet are pre-formed, meaning they are not produced as part of the disclosed method. Thus, disclosed are methods of producing a double-layer nanomatrix cell sheet comprising seeding fibroblasts on a nanomatrix fibroblast sheet; culturing the nanomatrix fibroblast sheet with seeded fibroblast to allow the seeded fibroblasts to spread on the top of nanomatrix fibroblast sheet, then depositing a nanomatrix SMC sheet on top of the seeded fibroblasts followed by culturing, thereby forming a double-layer nanomatrix cell sheet. In some aspects, the culture time is in the range of 1-30 days. In some aspect, the method can further comprise seeding SMCs on the double-layer nanomatrix cell prior to EC seeding. In some aspect, the seeding density for fibroblasts is in the range between 0.01 million cells/cm² to 1 million cells/cm². In some aspects, the culture duration for double-layer nanomatrix cell sheet with seeded SMCs is in the range of 2-32 days prior to EC seeding. In some aspects, the culture duration for double-layer nanomatrix cell sheet with seeded SMCs is in the range of 1-24 hours prior to EC seeding.

In some aspects, a nanomatrix fibroblast sheet is pre-formed, meaning it is not produced as part of the disclosed method. Thus, disclosed are methods of producing a double-layer nanomatrix cell sheet comprising seeding fibroblasts on a nanomatrix fibroblast sheet; culturing the nanomatrix fibroblast sheet containing seeded fibroblasts to allow the seeded fibroblasts to spread on the top of nanomatrix fibroblast sheet, then producing a Nanomatrix SMC sheet on top of the seeded fibroblasts, and followed by culturing, thereby forming a double-layer nanomatrix cell sheet. In some aspect, the seeding density for fibroblasts is in the range between 0.01 million cells/cm² to 1 million cells/cm². In some aspects, the culture duration for nanomatrix fibroblast sheet with seeded fibroblast is in the range of 1-24 hours prior to producing the nanomatrix SMC sheet. In some aspects, the culture time is in the range of 1-30 days. In some aspect, the method can further comprise seeding SMCs on the double-layer Nanomatrix cell sheet prior to EC seeding. In some aspect, the seeding density for SMCs is in the range between 0.01 million cells/cm² to 1 million cells/cm². In some aspects, the culture duration for double-layer nanomatrix cell sheet with seeded SMCs is in the range of 2-32 days prior to EC seeding. In some aspects, the culture duration for double-layer nanomatrix cell sheet with seeded SMCs is in the range of 1-24 hours prior to EC seeding.

In some aspects, a nanomatrix fibroblast sheet is pre-formed meaning it is not produced as part of the disclosed method. Thus, disclosed are methods of producing a double-layer nanomatrix sheet comprising seeding fibroblasts on a nanomatrix fibroblast sheet, culturing the nanomatrix fibroblast sheet with seeded fibroblast to allow the seeded fibroblasts to spread on the top of nanomatrix fibroblast sheet, and producing a Nanomatrix SMC sheet on top of the seeded fibroblasts. In some aspects, the method can further comprise culturing to form a double-layer nanomatrix cell sheet. In some aspect, the seeding density for fibroblasts is in the range between 0.01 million cells/cm² to 1 million cells/cm². In some aspects, the culture duration for nanomatrix fibroblast sheet with seeded fibroblast is in the range of 1-48 hours prior to producing the nanomatrix SMC sheet. In some aspects, the culture time is in the range of 2-30 days. In some aspect, the method can further comprise seeding SMCs on the double-layer Nanomatrix cell sheet prior to EC seeding. In some aspect, the seeding density for fibroblasts is in the range between 0.01 million cells/cm² to 1 million cells/cm². In some aspects, the culture duration for double-layer nanomatrix cell sheet with seeded SMCs is in the range of 1-48 hours or 2 to 32 days prior to EC seeding.

In some aspects, the nanomatrix SMC sheet is pre-formed, meaning it is not produced as part of the disclosed method. Thus, disclosed are methods of producing a double-layer nanomatrix cell sheet combining fibroblast cells with a peptide amphiphile solution first and then spread a layer of peptide amphiphile solution containing fibroblast cells over a substrate, and crosslinking the peptide amphiphiles, thereby forming a nanomatrix fibroblast sheet, seeding fibroblasts on the nanomatrix fibroblast sheet, culturing the nanomatrix fibroblast sheet with seeded fibroblast to allow the seeded fibroblasts to spread on the top of nanomatrix fibroblast sheet, depositing a nanomatrix SMC sheet on top of the seeded fibroblasts, followed by culturing, thereby forming a double-layer nanomatrix cell sheet. In some aspects, the method can further comprise seeding SMCs on the double-layer Nanomatrix cell sheet. In some aspect, the fibroblast density for producing nanomatrix fibroblast sheet is in a range between 0.05 million cells/cm² to 5 million cells/cm², the seeding density for fibroblasts on the nanomatrix fibroblast sheet is in the range between 0.01 million cells/cm² to 1 million cells/cm², and the seeding density for SMC on the nanomatrix SMC sheet is in the range of 0.01 million cells/cm² to 1 million cells/cm². In some aspects, the culture time after deposition of nanomatrix SMC sheet for forming double-layer nanomatrix sheet is in the range of 1-24 hours, or 2 to 30 days.

In some aspects, the culturing and seeding steps as described herein for forming the Nanomatrix fibroblast sheet and nanomatrix SMC sheet as part of the method can also be performed with the methods of using the pre-formed sheets.

In some aspects, the fibroblasts and SMCs are the same as described herein for forming the nanomatrix fibroblast sheet and nanomatrix SMC sheet as part of the method.

b. Forming the Nanomatrix Fibroblast Sheet and Nanomatrix SMC Sheet

Disclosed are methods of producing a double-layer nanomatrix cell sheet comprising producing a nanomatrix fibroblast sheet and a nanomatrix SMC sheet as part of the method. Thus, disclosed are methods of producing a double-layer nanomatrix cell sheet comprising combining fibroblast cells with a peptide amphiphile solution first and then spread a layer of peptide amphiphile solution containing fibroblast cells over a substrate, then crosslinking the peptide amphiphiles, thereby forming a nanomatrix fibroblast sheet, thereby forming a nanomatrix fibroblast sheet, seeding fibroblasts on the Nanomatrix fibroblast sheet; culturing the nanomatrix fibroblast sheet with seeded fibroblast cells to allow the seeded fibroblast cells to spread on the top of nanomatrix fibroblast sheet, and producing a nanomatrix SMC sheet on top of the seeded fibroblasts, thereby forming a double-layer nanomatrix cell sheet. In some aspects, producing a Nanomatrix SMC sheet on top of the seeded fibroblasts of the nanomatrix fibroblast sheet comprises combining SMCs with a peptide amphiphile solution first and then spread a layer of peptide amphiphile solution containing SMCs over the nanomatrix fibroblast sheet with seeded fibroblasts, then crosslinking the peptide amphiphiles and followed by culturing.

In some aspects, before seeding with fibroblasts, the methods further comprise culturing the nanomatrix fibroblast sheet in fibroblast complete expansion medium with 20% FBS and 1% penicillin-streptomycin to allow for cell spreading and remodeling within the nanomatrix and sheet maturation. In some aspects, the culturing duration can be 1-30 days. In some aspects, the culture medium comprises rhFGF, rhinsulin, L-glutamine, hydrocortisone hemisuccinate, ascorbic acid, and fetal bovine serum (FBS). In some aspects, the culture medium comprises FBS at a concentration of 20%. In some aspects, the culture medium comprises ascorbic acid at a concentration of 100 to 500 μg/mL. In some aspects, the culture medium is referred to as culture medium 1.

In some aspects, regardless of whether the method uses a pre-formed nanomatrix fibroblast sheet and/or pre-formed Nanomatrix SMC sheet or produces both as part of the method, the seeded fibroblasts act as a glue between the Nanomatrix fibroblast sheet and the Nanomatrix SMC sheet. In some aspects, after seeding fibroblasts on the nanomatrix fibroblast sheet, the fibroblasts, along with the nanomatrix fibroblast sheet, are cultured in culture medium. In some aspects, the culture medium is culture medium 1. In some aspects, culture duration is in the range of 1-48 hours. In some aspects, the nanomatrix fibroblast sheet is cultured with the seeded fibroblasts for 6 hours. In some aspect, the seeding density for fibroblasts is in the range between 0.01 million cells/cm² to 1 million cells/cm². In some aspects, the culture duration for double-layer nanomatrix cell sheet maturation is in the range of 1-30 days prior to SMC seeding.

In some aspects, the methods of producing a double-layer nanomatrix cell sheet further comprises seeding SMCs on the nanomatrix SMC sheet of the double-layer Nanomatrix cell sheet. In some aspect, the seeding density for SMCs is in the range between 0.01 million cells/cm² to 1 million cells/cm². In some aspects, the methods further comprise culturing the double-layer nanomatrix cell sheet containing seeded SMCs prior to seeding with ECs. In some aspects, the double-layer Nanomatrix cell sheet containing seeded SMCs is cultured in a culture medium. In some aspects, the culture medium is culture medium 2. In some aspects, the culture duration is in the range of 1-48 hours.

In some aspects, the culture medium is culture medium 2. In some aspects, culture medium 2 is a mix of two culture mediums, wherein the first culture medium comprises culture medium 1 (e.g. medium comprising rhFGF, rhinsulin, L-glutamine, hydrocortisone hemisuccinate, ascorbic acid, and fetal bovine serum (FBS)) and wherein the second culture medium is a primary smooth muscle cell expansion medium. In some aspects, the primary smooth muscle cell expansion medium comprises rhFGF, rhinsulin, L-glutamine, rhEGF, ascorbic acid, and FBS. In some aspects, culture medium 2 comprises FBS at a concentration of 20% in the first culture medium (e.g. culture medium 1). In some aspects, culture medium 2 comprises FBS at a concentration of 20% in the primary smooth muscle cell expansion medium. In some aspects, culture medium 2 comprises ascorbic acid at a concentration of 100 to 500 μg/mL in the first culture medium (e.g. culture medium 1). In some aspects, culture medium 2 comprises ascorbic acid at a concentration of 100 to 500 μg/mL in the primary smooth muscle cell expansion medium. In some aspects, the mixture of the two culture mediums (culture medium 1 and primary smooth muscle cell expansion medium) is different ratios, including 1:1, 1:2, 1:3, 1:4, 4:1, 3:1, 2:1, 1:1. Thus, in some aspects, culture medium 2 can be used to culture the double-layer Nanomatrix cell sheet for maturation and to culture the double-layer Nanomatrix cell sheet after seeding with SMCs.

In some aspects, regardless of whether the method uses a pre-formed nanomatrix fibroblast sheet and/or pre-formed nanomatrix SMC sheet or produces both as part of the method, the double-layer nanomatrix cell sheet can comprise SMCs in between the nanomatrix fibroblast sheet and the nanomatrix SMC sheet. Thus, in some aspects, instead of seeding fibroblasts on the nanomatrix fibroblast sheet, SMCs can be seeded on the nanomatrix fibroblast sheet. In some aspects, the nanomatrix fibroblast sheet with seeded SMCs are cultured for 1-48 hours in culture medium 2. In some aspects, SMCs can be used as the glue between the nanomatrix fibroblast sheet and the Nanomatrix SMC sheet.

In some aspects, the fibroblasts can be, but are not limited to, human aortic adventitial fibroblasts, primary human coronary artery fibroblasts, primary human cardiac fibroblasts, primary human brain vascular fibroblasts, primary human carotid artery fibroblast, primary pulmonary vein fibroblast, or primary human vein fibroblasts.

In some aspects, the smooth muscle cells can be, but are not limited to, primary human umbilical artery SMCs, primary human coronary artery SMCs, primary human pulmonary artery SMCs, primary human carotid artery SMCs, primary human brain vascular SMCs, primary human vein SMCs, primary human aortic SMCs, or human stem cell-derived SMCs.

In some aspects, the peptide amphiphile solution comprises one or more of the peptide Amphiphiles described herein and water. In some aspects, the peptide amphiphile solution comprises peptide amphiphiles comprising a hydrophobic tail and a hydrophilic peptide sequence. For example, In some aspects, the peptide amphiphile is PA-GTAGLIGQ-RGDS or a combination of PA-GTAGLIGQ-RGDS with other types of peptide amphiphiles. In some aspects, the peptide amphiphile is a combination of PA-GTAGLIGQ-RGDS and at least one PA selected from the group of PA-GTAGLIGQ-YIGSR, PA-KKKKK-GTAGLIGQ-YIGSR, PA-KKKKK-GTAGLIGQ-YIGSR-NO, PA-KKKKK-GTAGLIGQ, and PA-KKKKK-GTAGLIGQ-NO. In some aspects, the peptide amphiphile solution can further comprise one or more proteins or biomolecules typically found in the extracellular matrix, including but not limited to, collagen, elastin, fibronectin, hyaluronic acid, chondroitin sulfate, heparan sulfate, and proteoglycans.

In some aspects, the crosslinking of the peptide amphiphiles in producing the nanomatrix fibroblast sheet and/or nanomatrix SMC sheet is performed in the presence of calcium chloride. In some aspects, crosslinking occurs by misting the peptide amphiphiles with calcium chloride. In some aspects, the cross-linking agent can be, but is not hinted to CaCl₂), MgCL₂, ZnCL₂, ALCL₃ and FeCL₃. In some aspects, the CaCl₂ can be in the form of a mist or a solution. In some aspects, the concentration of CaCl₂ can range from 0.01M to 0.1M. In some aspects, a humidifier can be used to generate a CaCl₂ mist, which is then applied to the cells along with the peptide amphiphile solution. In some aspects, a spray bottle can be used to apply a CaCl₂ solution to the cells with the peptide amphiphile solution.

In some aspects, when including each of the culturing steps, disclosed are methods of producing a double-layer nanomatrix cell sheet comprising combining fibroblast cells with a peptide amphiphile solution first and then spread a layer of peptide amphiphile solution containing fibroblast cells over a substrate, and crosslinking the peptide amphiphiles, thereby forming a nanomatrix fibroblast sheet; culturing the nanomatrix fibroblast sheet in a culture medium to form a nanomatrix fibroblast sheet; seeding fibroblasts on the nanomatrix fibroblast sheet, thereby forming a fibroblast seeded nanomatrix fibroblast sheet; culturing the fibroblast seeded nanomatrix fibroblast sheet in a culture medium; producing a Nanomatrix SMC sheet on top of the seeded fibroblasts, thereby forming a double-layer nanomatrix cell sheet; culturing the double-layer nanomatrix cell sheet in a culture medium to form a double-layer nanomatrix cell sheet; seeding SMCs on the double-layer Nanomatrix cell sheet, thereby forming a smooth muscle cell seeded double-layer nanomatrix cell sheet; and culturing the smooth muscle cell seeded double-layer nanomatrix cell sheet in a culture medium. In some aspects, producing a Nanomatrix SMC sheet on top of the seeded fibroblasts comprises combining SMCs with a peptide amphiphile solution first and then spread a layer of peptide amphiphile solution containing SMCs over the nanomatrix fibroblast sheet with seeded fibroblasts, and then crosslinking the peptide amphiphiles. The method can further comprise culturing step to form a double-layer nanomatrix cell sheet.

iii. Producing Trilayer Nanomatrix Vascular Sheet

In some aspects, a trilayer Nanomatrix cell sheet is a nanomatrix vascular sheet.

In some aspects a nanomatrix vascular sheet can be made using a combination of two or more of the methods of producing single-layer and/or double-layer Nanomatrix cell sheets disclosed. In some aspects, different layers can be made separately and then combined to form the trilayer Nanomatrix. In some aspects, all layers are produced as part of the disclosed methods.

a. Starting with Pre-Formed Nanomatrix Fibroblast Sheet and/or Nanomatrix SMC Sheet

Disclosed are methods of producing a nanomatrix vascular sheet comprising seeding fibroblasts on a nanomatrix fibroblast sheet; optionally, culturing the nanomatrix fibroblast sheet containing seeded fibroblast, depositing a nanomatrix SMC sheet on top of the seeded fibroblasts, thereby forming a double-layer nanomatrix cell sheet; culturing the double-layer nanomatrix cell sheet, seeding SMCs on the double-layer nanomatrix cell sheet; culturing double-layer nanomatrix cell sheet with seeded SMCs, and seeding endothelial cells on top of the seeded SMCs, and followed by culturing to form trilayer nanomatrix vascular sheet. In some aspects, the nanomatrix fibroblast sheet and/or the nanomatrix SMC sheet are pre-formed and therefore are not produced as part of the disclosed method. In some aspect, the seeding density for fibroblasts is in the range between 0.01 million cells/cm² to 1 million cells/cm². In some aspects, the culture duration for forming a double-layer nanomatrix cell sheet is in the range of 1-30 days prior to SMC seeding. In some aspect, the seeding density for SMCs is in the range between 0.01 million cells/cm² to 1 million cells/cm². In some aspects, the double-layer nanomatrix sheet with seeded SMCs are cultured for 1-48 hours prior to EC seeding. In some aspects, the density for EC seeding is in the range of 0.01 million cells/cm² to 2 million cells/cm². In some aspects, the culture duration for forming triple nanomatrix vascular sheet is in the range of 1-30 days.

In some aspects, the nanomatrix fibroblast sheet is pre-formed, meaning it is not produced as part of the disclosed method. Thus, disclosed are methods of producing a nanomatrix vascular sheet comprising seeding fibroblasts on a nanomatrix fibroblast sheet; culturing the nanomatrix fibroblast sheet containing seeded fibroblast, producing a Nanomatrix SMC sheet on top of the seeded fibroblasts and followed by culturing to form a double-layer nanomatrix sheet, seeding SMCs on the double-layer Nanomatrix cell sheet; culturing the double-layer cell sheet with SMCs, and seeding endothelial cells on top of the seeded SMCs and followed by culturing to form a trilayer Nanomatrix vascular sheet. In some aspect, the seeding density for fibroblasts is in the range between 0.01 million cells/cm² to 1 million cells/cm². In some aspects, the culture duration for forming a double-layer nanomatrix is in the range of 1-30 days prior to SMC seeding. In some aspect, the seeding density for SMCs is in the range between 0.01 million cells/cm² to 1 million cells/cm² In some aspects, the double-layer nanomatrix cell sheet with seeded SMCs are cultured on the Nanomatrix fibroblast sheet for 1-48 hours prior to EC seeding. In some aspects, the density for EC seeding is in the range of 0.01 million cells/cm² to 2 million cells/cm². In some aspects, the culture duration after EC seeding for forming triple nanomatrix vascular sheet is in the range of 1-30 days.

In some aspects, the nanomatrix SMC sheet is pre-formed, meaning it is not produced as part of the disclosed method. Thus, disclosed are methods of producing a nanomatrix vascular sheet comprising combining fibroblast cells with a peptide amphiphile solution, applying a layer of peptide amphiphile solution containing fibroblast cells over a substrate, and crosslinking the peptide amphiphiles, followed by culturing to form nanomatrix fibroblast sheet, seeding fibroblasts on the nanomatrix fibroblast sheet; culturing the nanomatrix fibroblast sheet with seeded fibroblasts, depositing a nanomatrix SMC sheet on top of the seeded fibroblasts, followed by culturing to form double-layer nanomatrix cell sheet, seeding SMCs on the double-layer nanomatrix cell sheet; culturing the double-layer nanomatrix cell sheet seeded with SMCs, and seeding endothelial cells on top of the seeded SMCs on the double-layer nanomatrix cell sheet, and followed by culturing to form the trilayer nanomatrix vascular sheet. In some aspect, the fibroblast density for producing nanomatrix fibroblast sheet is in a range between 0.05 million cells/cm² to 5 million cells/cm². In some aspects, the culture duration for forming nanomatrix fibroblast sheet is in the range of 1-30 days. In some aspects, the seeding density for fibroblasts is in the range between 0.01 million cells/cm² to 1 million cells/cm². In some aspects, the culture duration for seeded fibroblasts prior to depositing a nanomatrix SMC sheet is in the range of 1-48 hours. In some aspects, the culture duration for forming double-layer nanomatrix cell sheet is in the range of 1-30 days prior to SMC seeding. In some aspect, the seeding density for SMCs is in the range between 0.01 million cells/cm² to 1 million cells/cm² In some aspects, the double layer nanomatrix cell sheet with seeded SMCs are cultured for 1-48 hours prior to EC seeding. In some aspects, the density for EC seeding is in the range of 0.01 million cells/cm² to 2 million cells/cm². In some aspects, the culture duration for forming triple nanomatrix vascular sheet after EC seeding is in the range of 1-30 days.

In some aspects, the culturing and seeding steps as described herein for forming the Nanomatrix fibroblast sheet and a nanomatrix SMC sheet as part of the method can also be performed with the methods of using the pre-formed sheets. In some aspects, the culturing steps can be performed as described for the methods including forming the nanomatrix fibroblast sheet and nanomatrix SMC sheet.

In some aspects, the fibroblasts and SMCs are the same as described herein for forming the Nanomatrix fibroblast sheet and a nanomatrix SMC sheet as part of the method.

b. Forming the Nanomatrix Fibroblast Sheet and Nanomatrix SMC Sheet

Disclosed are methods of producing a nanomatrix vascular sheet comprising combining fibroblast cells with a peptide amphiphile solution first and then spread a layer of peptide amphiphile solution containing fibroblast cells over a substrate, crosslinking the peptide amphiphiles, followed by culturing to form nanomatrix fibroblast sheet, seeding fibroblasts on nanomatrix fibroblast sheet; producing a Nanomatrix SMC sheet on top of the seeded fibroblasts, followed by culturing to form double-layer nanomatrix cell sheet, seeding SMCs on the double-layer Nanomatrix cell sheet; culturing the SMC seeded double-layer nanomatrix cell sheet; and seeding endothelial cells on top of the seeded SMCs, and followed by culturing to form a trilayer nanomatrix vascular sheet. In some aspects, producing a Nanomatrix SMC sheet on top of the seeded fibroblasts comprises combining SMCs with a peptide amphiphile solution first and applying a layer of peptide amphiphile solution containing SMCs over the fibroblast seeded nanomatrix fibroblast sheet, and then crosslinking the peptide amphiphiles, and followed by culturing, thereby forming a double-layer nanomatrix cell sheet, wherein the double-layer nanomatrix cell sheet comprises a nanomatrix smooth muscle sheet and a nanomatrix fibroblast sheet. In some aspects, culturing the nanomatrix fibroblast sheet in fibroblast complete expansion medium with FBS to allow for cell spreading and remodeling and sheet maturation before seeding the Nanomatrix fibroblast sheet with fibroblasts. In some aspects, this culturing step can allow the fibroblasts within the Nanomatrix fibroblast sheet to spread throughout to form nanomatrix fibroblast sheet. In some aspects, the culture medium comprises rhFGF, rhinsulin, L-glutamine, hydrocortisone hemisuccinate, ascorbic acid, and fetal bovine serum (FBS). In some aspects, the culture medium comprises FBS at a concentration range of 20 to 50%. In some aspects, the culture medium comprises ascorbic acid at a concentration range of 100 to 500 μg/mL. In some aspects, the culture medium is referred to as culture medium 1.

In some aspects, the methods further comprise culturing the nanomatrix fibroblast sheet after seeding with fibroblasts and prior to depositing a nanomatrix SMC sheet or producing a nanomatrix SMC sheet on top. Thus, in some aspects, after seeding fibroblasts on the nanomatrix fibroblast sheet, the fibroblasts, along with the nanomatrix fibroblast sheet, are cultured in culture medium. In some aspects, the culture medium comprises rhFGF, rhinsulin, L-glutamine, hydrocortisone hemisuccinate, ascorbic acid, and fetal bovine serum (FBS). In some aspects, the culture medium comprises FBS at a concentration range of 20 to 50%. In some aspects, the culture medium comprises ascorbic acid at a concentration range of 100 to 500 μg/mL. Thus, in some aspects, the culture medium is culture medium 1. In some aspects, culturing the nanomatrix fibroblast sheet, with the seeded fibroblasts, is conducted for 1-48 hours in culture medium prior to producing or depositing a nanomatrix SMC sheet. In some aspects, the nanomatrix fibroblast sheet is cultured with the seeded fibroblasts for 1-48 hours. In some aspects, the nanomatrix fibroblast sheet is cultured with the seeded fibroblasts for 6 hours.

In some aspects, the seeded fibroblasts act as a glue between the Nanomatrix fibroblast sheet and the Nanomatrix SMC sheet. In some aspects, the seeding density for fibroblasts is in the range between 0.01 million cells/cm² to 1 million cells/cm².

In some aspects, the methods further comprise culturing the double layer nanomatrix cell sheet after depositing a nanomatrix SMC sheet or producing a nanomatrix SMC sheet on top of the seeded fibroblasts and prior to seeding the SMCs on the double-layer nanomatrix cell sheet. Thus, in some aspects, immediately after depositing a nanomatrix SMC sheet or producing a nanomatrix SMC sheet on top of the seeded fibroblasts, the double-layer Nanomatrix cell sheet is cultured in culture medium. In some aspects, culturing the double-layer nanomatrix cell sheet is conducted for 1-30 days in culture medium. In some aspects, the culture medium is culture medium 2. In some aspects, culture medium 2 is a mix of two culture mediums, wherein the first culture medium comprises culture medium 1 (e.g. medium comprising rhFGF, rhinsulin, L-glutamine, hydrocortisone hemisuccinate, ascorbic acid, and fetal bovine serum (FBS)) and wherein the second culture medium is a primary smooth muscle cell expansion medium. In some aspects, the primary smooth muscle cell expansion medium comprises rhFGF, rhinsulin, L-glutamine, rhEGF, ascorbic acid, and FBS. In some aspects, culture medium 2 comprises FBS at a concentration of 20% in the first culture medium (e.g. culture medium 1). In some aspects, culture medium 2 comprises FBS at a concentration range of 20 to 50% in the primary smooth muscle cell expansion medium. In some aspects, culture medium 2 comprises ascorbic acid at a concentration range of 100 to 500 μg/mL in the first culture medium (e.g. culture medium 1). In some aspects, culture medium 2 comprises ascorbic acid at a concentration range of 100 to 500 μg/mL in the primary smooth muscle cell expansion medium. In some aspects, the mixture of the two culture mediums (culture medium 1 and primary smooth muscle cell expansion medium) is different ratios, including, a 1:1, 1:2, 1:3, 1:4, 1:5; 5:1 4:1, 3:1, 2:1.

In some aspects, the methods further comprise culturing the double-layer nanomatrix cell sheet after seeding SMCs on the double-layer nanomatrix cell sheet and prior to seeding with ECs. Thus, in some aspects, immediately after seeding the SMCs on the double-layer Nanomatrix cell sheet, the double-layer Nanomatrix cell sheet is cultured in culture medium. In some aspects, the culture medium is culture medium 2. In some aspects, culturing the double-layer nanomatrix cell sheet with the seeded SMCs is conducted for 1-48 hours in culture medium. In some aspects, the culturing is for 6 hours. In some aspects, the culture medium is culture medium 2. In some aspects, culture medium 2 is a mix of two culture mediums, wherein the first culture medium comprises culture medium 1 (e.g. medium comprising rhFGF, rhinsulin, L-glutamine, hydrocortisone hemisuccinate, ascorbic acid, and fetal bovine serum (FBS)) and wherein the second culture medium is a primary smooth muscle cell expansion medium. In some aspects, the primary smooth muscle cell expansion medium comprises rhFGF, rhinsulin, L-glutamine, rhEGF, ascorbic acid, and FBS. In some aspects, culture medium 2 comprises FBS at a concentration of 20% in the first culture medium (e.g. culture medium 1). In some aspects, culture medium 2 comprises FBS at a concentration range of 20 to 50% in the primary smooth muscle cell expansion medium. In some aspects, culture medium 2 comprises ascorbic acid at a concentration range of 100 to 500 μg/mL in the first culture medium (e.g. culture medium 1). In some aspects, culture medium 2 comprises ascorbic acid at a concentration range of 100 to 500 μg/mL in the primary smooth muscle cell expansion medium. In some aspects, the mixture of the two culture mediums (culture medium 1 and primary smooth muscle cell expansion medium) is different ratios, including, 1:1, 1:2, 1:3, 1:4, 1:5; 5;1; 4:1, 3:1, 2:1 ratio. Thus, in some aspects, culture medium 2 can be used to culture the double-layer Nanomatrix cell sheet and to culture the double-layer Nanomatrix cell sheet after seeding with SMCs

In some aspects, the methods further comprise culturing the trilayer nanomatrix vascular sheet. Thus, in some aspects, immediately after seeding the ECs on the seeded SMCs, the trilayer nanomatrix vascular sheet is cultured in culture medium. In some aspects, the culture medium is culture medium 3. In some aspects, culture medium 3 comprises a mixture of two culture mediums, wherein the first culture medium culture medium 2 and wherein the second culture medium is an endothelial cell expansion medium. In some aspects, the endothelial cell expansion medium comprises rhFGF, ascorbic acid, hydrocortisone hemisuccinate, L-glutamine, rhIGF-1, rhEGF, rhVEGF, and heparin sulfate, and FBS. In some aspect, culture medium 3 comprises FBS at a concentration of 2-50% and ascorbic acid at a concentration of 50 to 500 μg/mL in the endothelial cell expansion medium. In some aspects, the concentration of rhFGF is at a concentration of 5 ng/mL, the concentration of hydrocortisone hemisuccinate is 1 μg/mL, the concentration of L-glutamine is 10 mM, the concentration of rhEGF is 15 ng/mL, the concentration of rhVEGF is 5 ng/mL and the concentration of Heparin Sulfate is 0.75 U/mL. In some aspects, culture medium 3 is a mixture of the two culture mediums, culture medium 2 and endothelial cell expansion medium, in a 1:1, 1:2, 1:3, 1:4, 1:5; 5;1; 4:1, 3:1, 2:1 ratio, respectively.

In some aspects, culturing the trilayer nanomatrix vascular sheet is conducted for 1-30 days in culture medium 3.

Thus, when including each of the culturing steps, disclosed are methods of producing a nanomatrix vascular sheet comprising combining fibroblast cells with a peptide amphiphile solution, applying a layer of peptide amphiphile solution containing fibroblast cells over a substrate, and crosslinking the peptide amphiphiles, followed by culturing to form nanomatrix fibroblast sheet; seeding fibroblasts on the Nanomatrix fibroblast sheet; culturing the fibroblast seeded nanomatrix fibroblast sheet in a culture medium; producing a Nanomatrix SMC sheet on top of the seeded fibroblasts, followed by culturing in a culture medium to form a double-layer matrix sheet, seeding SMCs on the double-layer Nanomatrix cell sheet, thereby forming a smooth muscle cell seeded double-layer nanomatrix cell sheet; culturing the smooth muscle cell seeded double-layer nanomatrix cell sheet in a culture medium; seeding endothelial cells on top of the smooth muscle cell seeded double-layer nanomatrix cell sheet, followed by culturing it in a culture medium, thereby producing a trilayer nanomatrix vascular sheet.

In some aspects, producing a Nanomatrix SMC sheet on top of the seeded fibroblasts, comprises combining SMCs with a peptide amphiphile solution first and then spread a layer of peptide amphiphile solution containing SMCs over the nanomatrix fibroblast sheet with seeded fibroblasts, and then crosslinking the peptide amphiphiles and followed by culturing, thereby forming a double-layer nanomatrix cell sheet.

In some aspects, the fibroblasts can be, but are not limited to, human aortic adventitial fibroblasts, primary human coronary artery fibroblasts, primary human cardiac fibroblasts, primary human brain vascular fibroblasts, primary human carotid artery fibroblast, primary pulmonary vein fibroblast, or primary human vein fibroblasts.

In some aspects, the smooth muscle cells can be, but are not limited to, primary human umbilical artery SMCs, primary human coronary artery SMCs, primary human pulmonary artery SMCs, primary human carotid artery SMCs, primary human brain vascular SMCs, primary human vein SMCs, primary human aortic SMCs, or human stem cell-derived SMCs.

In some aspects, the endothelial cells can be, but are not limited to, human primary endothelial cells. In some aspects, the human primary endothelial cells are primary human umbilical vein ECs, primary human coronary artery ECs, primary human pulmonary artery ECs, primary human carotid artery ECs, primary human brain microvascular ECs, primary human vein ECs, primary human aortic ECs, mesenchymal stem cell-derived ECs, human induced pluripotent stem cell-derived ECs, or human embryonic stem cell-derived ECs.

In some aspects, the peptide amphiphile solution comprises one or more of the peptide Amphiphiles described herein and. In some aspects, the peptide amphiphile is PA-GTAGLIGQ-RGDS or a combination of PA-GTAGLIGQ-RGDS with other types of peptide amphiphiles. In some aspects, the peptide amphiphile is a combination of PA-GTAGLIGQ-RGDS and at least one PA selected from the group of PA-GTAGLIGQ-YIGSR, PA-KKKKK-GTAGLIGQ-YIGSR, PA-KKKKK-GTAGLIGQ-YIGSR-NO, PA-KKKKK-GTAGLIGQ, and PA-KKKKK-GTAGLIGQ-NO. In some aspects, the peptide amphiphile solution can further comprise one or more proteins or biomolecules typically found in the extracellular matrix, including but not limited to, collagen, elastin, fibronectin, hyaluronic acid, chondroitin sulfate, heparan sulfate, and proteoglycans.

In some aspects, the crosslinking of the peptide amphiphiles in producing the nanomatrix fibroblast sheet and/or nanomatrix SMC sheet is performed in the presence of calcium chloride. In some aspects, crosslinking occurs by misting the peptide amphiphiles with calcium chloride. In some aspects, the cross-linking agent can be, but is not hinted to CaCl₂, MgCL₂, ZnCL₂, ALCL₃ and FeCL₃. In some aspects, the CaCl₂ can be in the form of a mist or a solution. In some aspects, the concentration of CaCl₂ can range from 0.01M to 0.1M. In some aspects, a humidifier can be used to generate a CaCl₂ mist, which is then applied to the cells along with the peptide amphiphile solution. In some aspects, a spray tool, such as a spray bottle, can be used to apply a CaCl₂ solution to the cells with the peptide amphiphile solution.

In some aspects, the culturing can be under either static or dynamic conditions. In some aspects, culturing in a multi-well plate, petri dish, or home-made 3D printed chamber or chip, no flow stimulation is used. In some aspects, culturing uses a perfusable bioreactor system that provides physiological laminar flow and pressure. In some aspects, this system can help to generate a microenvironment that mimics or reproduces the conditions that are present in native tissue architecture in vitro. In some aspects, the multi-well plate, petri dish, or home-made 3D printed chamber or chip containing the nanomatrix cell sheets, including single layer nanomatrix cell sheet, double layer nanomatrix cell sheet, or triple layer nanomatrix vascular sheet, is connected to the bioreactor, which can deliver growth medium, nutrients, and factors to the wells or channels of the multi-well plate.

### 2. Methods of Inducing One or More Atherosclerosis Features on Trilayer Nanomatrix Vascular Sheet

After producing a trilayer Nanomatrix cell sheet, specifically a Nanomatrix vascular sheet, as disclosed throughout, atherosclerosis can be induced in the Nanomatrix vascular sheet.

In some aspects, any of the disclosed methods of producing trilayer Nanomatrix cell sheets, or Nanomatrix vascular sheets, can further comprise the following steps to induce nanomatrix vascular sheet with one or more key atherosclerotic features.

i. Inducing Endothelial Cell Dysfunction

In some aspects, after producing the Nanomatrix vascular sheet, the methods can further comprise inducing endothelial cell dysfunction to initiate atherosclerosis on the nanomatrix vascular sheet. In some aspects, inducing endothelial cell dysfunction comprises culturing the nanomatrix vascular sheet in a culture medium comprising Dulbecco's Modified Eagle Medium (DMEM), TNF-α, Ox-LDL, and serum. In some aspects, the concentration of TNF-α is 10 ng/mL to 500 ng/mL. In some aspects, the concentration of TNF-α is 40 ng/mL. In some aspects, the concentration of Ox-LDL is 15 μg/mL to 200 μg/mL. In some aspects, the concentration of Ox-LDL is 50 μg/mL. In some aspects, the concentration of serum is 0.10% to 2%. In some aspects, the concentration of serum is 0.5%. In some aspects, the serum is FBS. In some aspects, the culture medium can further comprise an antibiotic, such as but not limited to, penicillin-streptomycin. In some aspects, the culture medium comprises DMEM containing TNF-α (40 ng/mL), and Ox-LDL (50 μg/mL) and 0.5% FBS and 1% penicillin-streptomycin. In some aspects, the culture medium is referred to as culture medium 4. In some aspects, the culturing to induce endothelial cell dysfunction can comprises culturing for 1-24 hours, or 1-3 days. In some aspects, the nanomatrix vascular sheet after inducing endothelial cell dysfunction produces the earlier disclosed VS-ED. In some aspects, induction of endothelial cell dysfunction can be determined by determining the presence of endothelial dysfunction markers, such as but not limited to, ICAM-1, and vascular cell adhesion protein 1 (VCAM-1).

ii. Inducing Monocyte Recruitment

In some aspects, the methods can further comprise inducing monocyte recruitment to VS-ED. In some aspects, inducing monocyte recruitment occurs after inducing endothelial cell dysfunction as the dysfunctional endothelial cells can recruit the monocytes. In some aspects, inducing monocyte recruitment comprises culturing the nanomatrix vascular sheet, after inducing endothelial cell dysfunction, in a culture medium comprising DMEM, serum, and monocytes. In some aspects, the serum is a concentration of 0.1% to 2%. In some aspects, the serum is a concentration of 0.5%. In some aspects, the serum is FBS. In some aspects, the monocytes are a human monocyte cell line. In some aspects, the human monocyte cell line can be, but is not limited to, THP-1, U937, MM12, MUTZ-3, or MDM2. In some aspects, the monocytes are primary human monocytes. In some aspects, the monocytes are at a concentration of 0.5 million/mL to 5 million/mL. In some aspects, the monocytes are at a concentration of 1 million/mL. In some aspects, the culture medium is referred to as culture medium 5. In some aspects, the culturing to induce monocyte recruitment can comprising culturing for 1-48 hours or 1-3 days. In some aspects, the culturing to induce monocyte recruitment can comprising culturing for 6 hours to 2 days. In some aspects, the VS-ED after inducing monocyte recruitment produces the earlier disclosed VS-MO.

iii. Inducing Macrophage Formation

In some aspects, after inducing monocyte recruitment, the methods can further comprise inducing macrophage formation on the VS-MO. In some aspects, inducing macrophage formation comprises culturing the nanomatrix vascular sheet in a culture medium comprising DMEM, Ox-LDL, M-CSF, GM-CSF, IFN-γ, serum, and monocytes. In some aspects, the serum is a concentration of 0.1% to 2%. In some aspects, the serum is a concentration of 0.5%. In some aspects, the serum is FBS. In some aspects, the GM-CSF is a concentration of 20 ng/mL to 500 ng/mL. In some aspects, the GM-CSF is a concentration of 25 ng/mL. In some aspects, the M-CSF is a concentration of 20 ng/mL to 500 ng/mL. In some aspects, the IFN-γ is a concentration of 50 ng/mL to 500 ng/mL. In some aspects, the IFN-γ is a concentration of 100 ng/mL. In some aspects, the monocytes are at a concentration of at least 0.1 million/mL to 3 million/mL. In some aspects, the Ox-LDL is at a concentration of 15 μg/mL to 2000 μg/mL. In some aspects, the Ox-LDL is at a concentration of 50 μg/mL. In some aspects, the culture medium is referred to as culture medium 6. In some aspects, the culturing to induce macrophage formation can comprise culturing for 1-21 days. In some aspects, the VS-MO after this step, inducing macrophage formation, produces the earlier disclosed VS-MA.

iv. Inducing Foam Cell and Fatty Streak Formation

In some aspects, after inducing macrophage formation, the methods further comprise inducing foam cell and fatty streak formation on VS-MA. In some aspects, inducing foam cell and fatty streak formation comprises culturing the nanomatrix vascular sheet in a culture medium comprising DMEM, Ox-LDL, M-CSF, GM-CSF, IFN-γ, serum, and monocytes. In some aspects, the culture medium can be the same as that used for inducing macrophage formation except the concentration range of the Ox-LDL is different. In some aspects, the serum is a concentration of 0.1% to 2%. In some aspects, the serum is a concentration of 0.5%. In some aspects, the serum is FBS. In some aspects, the GM-CSF is a concentration of 20 ng/ml to 500 ng/ml. In some aspects, the GM-CSF is a concentration of 25 ng/ml. In some aspects, the M-CSF is a concentration of 20 ng/mL to 500 ng/mL. In some aspects, the IFN-γ is a concentration of 50 ng/mL to 500 ng/mL. In some aspects, the monocytes are at a concentration of at least 0.1 million/mL to 3 million/mL. In some aspects, the monocyte concentration is 0.1 million/mL. In some aspects, the Ox-LDL is at a concentration of 100 μg/mL to 500 μg/mL. In some aspects, the culture medium is referred to as culture medium 7. In some aspects, the only difference between culture medium 6 and culture medium 7 is the concentration of Ox-LDL. In some aspects, the concentration of Ox-LDL in culture medium 6 is chosen from 15 μg/mL to 50 μg/mL, for example a concentration of at least 30 μg/mL. In some aspects, the concentration of Ox-LDL in culture medium 7 is selected from 100 μg/mL to 200 μg/mL, for example a concentration of 150 μg/mL. In some aspects, the culturing to induce foam cell formation can comprise culturing for 1-24 hours or 1-21 days. In some aspects, the VS-MA inducing foam cell and fatty streak formation produces the earlier disclosed nanomatrix vascular sheet with atherosclerosis (VS-AS).

In some aspects, one or more of the culturing steps is performed in a multi-well plate or petri dish or 3D printed chambers or chips. In some aspects, the multi-well plate is a 24-well plate, 48-well plate, 96-well plate, or 384 well plate. In some aspects, the multi-well plate can be printed with channels using Polydimethylsiloxane. In some aspects, the channels can be in the shape of, but not limited to, a square, rectangle, circle, oval, oblong, triangle, Y, or any combination of shapes.

In some aspects, the culturing can be under either static or dynamic conditions. In some aspects, culturing under no flow stimulation is used. In some aspects, culturing uses a perfusable bioreactor system that provides physiological laminar flow and pressure. In some aspects, this system can help to generate a microenvironment that mimics or reproduces the conditions that are present in native tissue architecture in vitro. In some aspects, the culturing uses a culturing system that provides disturbed flow. In some aspects, disturbed flow can be in the form of oscillating flow, which is generated and controlled by a bioreactor or a shaker. In some aspects, the oscillating flow creates a microenvironment that mimics the shear stress experienced by cells in atherosclerosis in vivo. In some aspects, the multi-well plate, petri dish, or home-made 3D printed chamber containing VS-DE, VS-MO, VS-MA, or VS-AS, is connected to the bioreactor or placed on a shake that supplied with atherosclerosis induced medium, such as culture medium 4, 5, 6, or 7.

In some aspects of the disclosed methods, the atherosclerotic nanomatrix vascular sheet comprises an inflammation-activated nanomatrix vascular sheet, a plaque, or both. In some aspects, an inflammation-activated nanomatrix vascular sheet refers to the trilayer Nanomatrix vascular sheet comprising an inflammation-activated nanomatrix fibroblast sheet, a layer of inflammation-activated fibroblast, an inflammation-activated nanomatrix SMC sheet, a layer of inflammation-activated SMCs, and a layer of dysfunctional ECs. In some aspects, the inflammation-activated nanomatrix fibroblast sheet comprises nanomatrix and inflammation-activated fibroblasts, wherein the inflammation-activated nanomatrix SMC sheet comprises nanomatrix and inflammation-activated SMCs. In some aspects, the nanomatrix fibroblast sheet and the nanomatrix SMC sheet become inflammation-activated nanomatrix fibroblast sheet and inflammation-activated nanomatrix SMC sheets once inducing atherosclerosis in the Nanomatrix vascular sheets. In some aspects, a plaque comprises one or more components, wherein the one or more components can be, but are not limited to, human monocyte, human macrophage, human EC, human fibroblast, human SMC, human foam cell, human low-density lipoproteins, cytokine, chemokine, reactive oxygen species, growth factors, fatty streak, and/or calcification.

In some aspects, a plaque refers to the presence of components such as human monocytes, human macrophages, human ECs, human fibroblasts, human SMCs, human foam cells, human low-density lipoproteins, cytokines, chemokines, reactive oxygen species, growth factors, fatty streak and/or calcification.

### 3. Methods of Inducing One or More Atherosclerosis Features on Single or Double Nanomatrix Cell Sheet

In some aspects, the methods for inducing endothelial cell dysfunction and monocyte recruitment on VS are used to produce inflammation-activated single-layer nanomatrix fibroblast or SMC sheet and induce monocyte recruitment on those sheet, thereby producing SL-MO.

In some aspects, the methods for inducing endothelial cell dysfunction and monocyte recruitment on VS can be used to produce inflammation-activated double-layer nanomatrix cell sheet and induce monocyte recruitment on the double-layer nanomatrix cell sheet, thereby producing DL-MO.

In some aspects, the methods for inducing macrophage formation on VS can be used to induce macrophage formation on single or double layer nanomatrix cell sheet, thereby producing SL-MA or DL-MA

In some aspects, the methods for inducing foam cell or fatty streak formation on VS can be used to inducing foam cell or fatty streak formation on single-layer or double-layer nanomatrix cell sheet, thereby producing SL-FC or DL-FC.

### 4. Methods of Screening

Disclosed are methods of using the disclosed nanomatrix vascular sheet to evaluate the therapeutic cytotoxicity (safety) or methods of using the disclosed nanomatrix vascular sheet or nanomatrix vascular sheet with one or more atherosclerosis features (VS-ED, VS-MR, VS-MF, or VS-AS) to identify therapeutic candidates for preventing or treating atherosclerosis.

i. Methods of Screening Therapeutic Cytotoxicity (Safety) Using VS-Based Assay

Disclosed are methods of making and using the disclosed VS-based assay to screen the cytotoxicity of therapeutic candidates. In some aspects, determining changes the cell viability by detecting activity of mitochondrial enzymes in live cells, the changes in the morphology of VS in response to a therapeutic candidate can be used to identify a therapeutic cytotoxicity.

a. Methods of Screening Therapeutic Cytotoxicity (Safety) Using VS-Based MTT Assay

Disclosed are methods of making and using the disclosed VS-based assay to screen the cytotoxicity of therapeutic candidates by determine the changes of cell viability via detecting the changes in the activity of mitochondrial enzymes in live cells of VS, comprising making VS in a multi-well plate, culturing VS in the presence of the therapeutic drug candidate of different concentrations in the multi-well plate; culturing a VS in the absence of the therapeutic candidate (control group) in the multi-well plate; detecting cell viability by detecting the activity of mitochondrial enzymes in live cells in VS cultured in the presence and absence of the therapeutic candidate; calculating the percentage of cell viability for each concentration compared to the control group (no candidate); wherein the highest concentration of the therapeutic candidate where cell viability remains near 100% is the highest non-cytotoxic concentration of the therapeutic; wherein the concentration of the candidate that reduces cell viability by 50% is the IC50 value of the therapeutic. The duration for culturing is in the range from 24-72 hours. In some respects, the multi-well plate can be, but not limited to, 48-well, 96-well, or 384-well plate.

b. Methods of Screening Therapeutic Cytotoxicity (Safety) Using VS-Based Live and Dead Cell Staining or Assay

Disclosed are the methods of making and using the disclosed VS-based assay to screen the cytotoxicity of therapeutic candidates by determine change in cell viability via measuring the intensity of live cell staining dye for live cells and dead cell staining dyes for dead cells of the VS, comprising making VS in a multi-well plate, culturing VS in the presence of the therapeutic drug candidate of different concentrations (treated groups) in the multi-well plate; culturing a VS in the absence of the therapeutic candidate (control group) in the multi-well plate; detecting cell viability by measuring the intensity of live cell staining dye for live cells and dead cell staining dyes for dead cells of the VS in the treated groups and control group; calculating the percentage of cell viability for treated group compared to the control groups; wherein the highest concentration of the therapeutic candidate where the ratio of live to total cells is near 100% is the highest non-cytotoxic concentration of the therapeutic. In some aspects, the live cell staining dye can be, but not limited to, Calcein-AM, Hoechst Dyes, CellTracker Green, and Fluorescein Diacetate. In some aspects, the dead cell staining dye can be, but not limited to, Ethidium Homodimer-1, Yo-Pro-1, Sytox Dyes, Propidium Iodide, and Annexin V. In some aspects, the method further comprising imaging the live cells and dead cells in the VS in the presence of therapeutic or absence of therapeutic using fluorescent microscope and employing image analysis software to quantify the number of live and dead cells in each image; wherein the highest concentration of the therapeutic candidate where the ratio of live to total cells is near 100% is the highest non-cytotoxic concentration of the therapeutic. In some aspects, the duration for culturing is in the range from 24-72 hours. In some respects, the multi-well plate can be, but not limited to, 48-well, 96-well, or 384-well plate.

ii. Methods of Screening Therapeutic Candidates to Identify Potential Therapeutic for Preventing or Treating Atherosclerosis Using VS-Based Assay
a. Methods of Screening Therapeutic Candidates to Identify Potential Therapeutic for Preventing Atherosclerosis Using VS

Disclosed are methods of using the disclosed VS to screen therapeutic candidate to identify potential therapeutics for preventing atherosclerosis development. In some aspects, the methods comprising determining the changes in EC activation, NO production, secretion of anti-or, -pro-inflammatory cytokines, ROS level, monocyte recruitment, monocyte-to-macrophage transition, monocyte apoptosis, macrophage polarization, scavenger receptor expression, phagocytosis, macrophage apoptosis, or autophagy in response to a therapeutic candidate to identify a potential therapeutic. In some aspects, the therapeutic candidates that can be screened with include but are not limited to naturally occurring substances such as herbs, plants, or minerals, synthesized chemical compounds in a laboratory to create new drugs, biological agents, lipids, proteins, peptides, antibodies, polysaccharides, nucleic acids such as microRNA, DNA, or siRNA. In some aspects, the potential therapeutics that can be identified using VS can be, but not limited to, endothelial cell activation modulators, nitric oxide (NO) pathway boosters, macrophage or monocyte apoptosis suppressor, antioxidant, or anti-inflammatory agents.

### (A) Methods of Identifying Endothelial Cell Activation Suppressor Using VS Based Assay

Disclosed are the methods of using the disclosed VS-based assay to identify Endothelial Cell Activation suppressor candidates for preventing atherosclerosis development, comprising making VS in a multi-well plate; culturing VS in the presence of the various therapeutic drug candidates of different concentrations (treated groups) in the multi-well plate; culturing a VS in the absence of the various therapeutic candidates (control group) in the multi-well plate; conducting endothelial activation marker analysis of the VS in protein level by performing immunostaining, western blotting, or both; imaging the VS and quantifying fluorescence intensity, protein band intensity, or both of those capture images using image analysis software, calculating the fold change in marker expression of treated groups compared to the untreated VS control; In some aspects, conducting endothelial activation marker analysis of the VS in protein level by quantifying pro-inflammatory cytokine release from the VS culture supernatant and calculating the fold change in amount of pro-inflammatory cytokine release from treated groups compared to the untreated VS control. In some respects, the method further comprises conducting quantitative PCR to measure expression of endothelial activation marker in gene level and calculating the fold change in marker expression of treated groups compared to the untreated VS control. In some aspects, the endothelial activation marker includes but not limited to, E-selectin, P-selectin, ICAM-1, VCAM-1, IL-1, IL-6, IL-8, MCP-1, and Nuclear Factor-KB. In some aspects, the therapeutic candidate result in statistically significant reduction in endothelial activation marker expression compared to the control in gene, protein or both levels is identified as a treatment as endothelial cell activation suppressor. In some respects, the multi-well plate can be, but not limited to, 48-well, 96-well, or 384-well plate. In some respects, the culture medium is selected from one or more from culture medium 4, 5, 6, or 7. In some aspects, the duration for culturing in culture medium 4, 5, 6, or 7 is selected from the range from 1-24 hours or 1 day to 60 days.

### (B) Methods of Identifying Nitric Oxide (NO) Pathway Boosters Using VS Based Assay

Disclosed are the methods of using the disclosed VS-based assay to identify nitric oxide (NO) pathway boosters for preventing atherosclerosis development, comprising making VS in a multi-well plate; culturing VS in the presence of the various therapeutic drug candidates of different concentrations (treated groups) in the multi-well plate; culturing a VS in the absence of the various therapeutic candidates (control group) in the multi-well plate; using nitric oxide detection reagents to measure intracellular, extracellular NO levels, or both levels, measure the fluorescent intensity or absorbance of the NO detection reagent using a fluorescence microplate reader or absorbance reader, and quantifying the increasing in NO levels between the control group and treated groups. In some aspects, the nitric oxide detection reagents includes, but not limited to, DAF-2 diacetate, DAF-FM, and Griess Reagent. In some respects, the method further comprises conducting quantitative PCR to measure expression of NO production and calculating the fold change in marker expression of treated groups compared to the untreated VS control. In some aspects, the therapeutic candidate resulted in statistically significant enhancing in NO production compared to the control is identified as a potential therapeutic as nitric oxide (NO) pathway boosters. In some respects, the multi-well plate can be, but not limited to, 48-well, 96-well, or 384-well microplate. In some respects, the culture medium is selected from one or more from culture medium 4, 5, 6, or 7. In some aspects, the duration for culturing in culture medium 4, 5, 6, or 7 is selected from the range from 1-24 hours or 1 day to 60 days.

### (C) Methods of Identifying Antioxidant Using VS Based Assay.

Disclosed are the methods of using the disclosed VS-based assay to identify antioxidant for preventing atherosclerosis development, comprising making VS in a multi-well plate; culturing VS in the presence of the various therapeutic drug candidates of different concentrations (treated groups) in the multi-well plate; culturing a VS containing TNF-alpha and Ox-LDL in the absence of the various therapeutic candidates (control group) in the multi-well plate; measuring intracellular ROS levels in VS using ROS detection reagents; quantifying the decreasing in ROS levels between the treated group and control group. In some aspects, the ROS detection reagents includes, but not limited to Dihydroethidium (DHE), 2′,7′-Dichlorodihydrofluorescein diacetate (DCFH-DA), CellROX Green Reagent, and Hydroxyphenyl fluorescein. In some aspects, the therapeutic candidate resulted in statistically significant reduction in ROS level compared to the control is as a treatment as antioxidant. In some respects, the multi-well plate can be, but not limited to, 48-well, 96-well, or 384-well plate. In some respects, the culture medium is selected from one or more from culture medium 4, 5, 6, or 7. In some aspects, the duration for culturing in culture medium 4, 5, 6, or 7 is selected from the range from 1-24 hours or 1 day to 60 days.

### (D) Methods of Identifying Anti-Inflammatory Agent Using VS Based Assay.

Disclosed are the methods of using the disclosed VS-based assay to identify anti-inflammatory agent for preventing atherosclerosis development, comprising making VS in a multi-well plate; culturing VS in the presence of the various therapeutic drug candidates of different concentrations (treated groups) in the multi-well plate; culturing a VS in the absence of the various therapeutic candidates (control group) in the multi-well plate; quantifying the reduction in cytokine levels by comparing the amount of pro-inflammatory cytokine in the treated group to the control group. In some aspects, the pro-inflammatory cytokines includes, IL-6, IL-1, IL-8, MCP-1, and IFN-γ. In some aspects, the therapeutic candidate resulted in statistically significant reduction in pro-inflammatory cytokines amount in the treated group compared to the control is identified as a treatment as anti-inflammatory agent. In some respects, the multi-well plate can be, but not limited to, 48-well, 96-well, or 384-well plate. In some respects, the culture medium is selected from one or more from culture medium 4, 5, 6, or 7. In some aspects, the duration for culturing in culture medium 4, 5, 6, or 7 is selected from the range from 1-24 hours or 1 day to 60 days.
(E) Methods of identifying therapeutics with bi or multifunction using VS based assay.

Disclosed are the methods of using the disclosed VS-based assay to identify therapeutics with bi or multifunction for preventing atherosclerosis development, comprising making VS in a multi-well plate; culturing VS in the presence of the various therapeutic drug candidates of different concentrations (treated groups) in the multi-well plate; culturing a VS in the absence of the various therapeutic candidates (control group) in the multi-well plate; quantifying two or more changes in the expression or production of markers, such as the endothelial activation markers, NO, ROS, or pro-inflammatory cytokines in treated group compared to the control group; the therapeutic candidate resulted in statistically significant changes in two or more markers preventing atherosclerosis development in the treated group compared to the control is identified as treatment as bi or multifunctional agent. In some respects, the multi-well plate can be, but not limited to, 48-well, 96-well, or 384-well plate. In some respects, the culture medium is selected from one or more from culture medium 4, 5, 6, or 7. In some aspects, the duration for culturing in culture medium 4, 5, 6, or 7 is selected from the range from 1-24 hours or 1 day to 60 days.

### (F) Methods of Identifying Therapeutics Using SL or DL Nanomatrix Sheet Based Assay

The disclosed methods above associated with VS can be applied to identify therapeutics in a less complicated environment and cellular components for evaluating therapeutic cytotxity or preventing atherosclerosis development using SL or DL nanomatrix sheet based assay for identifying therapeutics for preventing atherosclerosis development b. Methods of Screening Therapeutic Candidates to Identify Potential Therapeutic for Preventing Atherosclerosis Using VS-DE, VS-MO, or VS-MF based assay.

The disclosed methods above associated with VS can be applied to identify therapeutics in more complicated environment and cellular components for preventing atherosclerosis development using VS-ED for identifying therapeutics for preventing atherosclerosis development as endothelial cell activation modulators, monocyte recruitment, nitric oxide (NO) pathway boosters, antioxidant, anti-inflammatory agents, or bi- or multi-functional agents. The disclosed methods above associated with VS can be applied to identify therapeutics in a more complicated environment comprising more and cellular components for preventing atherosclerosis development using VS-MO for identifying therapeutics for preventing atherosclerosis development as endothelial cell activation modulators, nitric oxide (NO) pathway boosters, antioxidant, anti-inflammatory agents, or bi- or multi-functional agents. In some respects, the multi-well plate can be, but not limited to, 48-well, 96-well, or 384-well plate. In some respects, the culture medium is selected from one or more from culture medium 4, 5, 6, or 7. In some respects, the duration for culturing in culture medium 4, 5, 6, or 7 is selected from the range from 1-24 hours or 1 day to 60 days.

iii. Methods of Screening Therapeutic Efficacy for Treating Atherosclerosis Using VS-AS-Based Assay

Disclosed are methods of using the disclosed atherosclerotic nanomatrix vascular sheets (VS-AS) to test or identify therapeutic candidates for treat atherosclerosis. In some aspects, determining changes in foam cell amount, secretion of inflammatory or anti-inflammatory cytokines, and/or alterations in atherosclerosis-related gene expression in the atherosclerotic nanomatrix vascular sheets in response to a therapeutic candidate can be used to identify a treatment.

Disclosed are methods of screening for a therapeutic candidate comprising culturing an atherosclerotic nanomatrix vascular sheet in the presence of the therapeutic drug candidate; culturing an atherosclerotic nanomatrix vascular sheet in the absence of the therapeutic candidate; and detecting the presence of lipids in the atherosclerotic nanomatrix vascular sheet cultured in the presence and absence of the therapeutic candidate; wherein fewer lipids in the atherosclerotic nanomatrix vascular sheet cultured in the presence of the therapeutic candidate compared to the atherosclerotic nanomatrix vascular sheet cultured in the absence of the therapeutic candidate indicates the therapeutic candidate is effective at treating atherosclerosis.

In some aspects, detecting the presence of lipids comprises staining the atherosclerotic nanomatrix vascular sheet with a lipid-detection staining agent. In some aspects, the lipid-detection staining agent is BODIPY or Oil-Red-O. In some aspects, BODIPY or Oil-Red-O can be used to visualize foam cell formation. Thus, in some aspects, the disclosed methods, instead of detecting the presence of lipids, can also be used for detecting the presence of changes in foam cell formation. In some aspects, foam cell number can be reduced in the presence of a therapeutic candidate.

Disclosed are methods of screening for a therapeutic candidate comprising culturing an atherosclerotic nanomatrix vascular sheet in the presence of the therapeutic candidate; culturing an atherosclerotic nanomatrix vascular sheet in the absence of the therapeutic candidate; and detecting the amount of an atherosclerosis biomarker in the atherosclerotic nanomatrix vascular sheet cultured in the presence and absence of the therapeutic candidate; wherein a change in the amount of the atherosclerosis biomarker in the atherosclerotic nanomatrix vascular sheet cultured in the presence of the therapeutic drug candidate compared to the atherosclerotic nanomatrix vascular sheet cultured in the absence of the therapeutic candidate indicates the therapeutic candidate is effective at treating atherosclerosis.

In some aspects, the atherosclerosis biomarker is a cytokine, enzyme, gene that promotes atherosclerosis progression, or gene that suppresses atherosclerosis progression.

In some aspects, the cytokine is a pro-inflammatory cytokine. In some aspects, the proinflammatory cytokine is IL-1, IL-6, IL-8, IL-12, IFN-gamma, or MCP-1. In some aspects, the change in amount of pro-inflammatory cytokine that indicates a therapeutic drug is a decrease in pro-inflammatory cytokine. In some aspects, the cytokine is an anti-inflammatory cytokine. In some aspects, the anti-inflammatory cytokine can be, but is not limited to, IL-13, IL-4, IL-10, or IL-5. In some aspects, the change in amount of anti-inflammatory cytokine that indicates a therapeutic drug is an increase in pro-inflammatory cytokine.

In some aspects, the enzyme is matrix metalloproteinases, lipoprotein-associated phospholipase A2, or cyclooxygenase. In some aspects, the change in amount, activity or both of enzyme that indicates a therapeutic drug is a decrease in enzyme amount, activity, or both.

In some aspects, a therapeutic candidate that downregulates the expression of genes that promote atherosclerosis progression and/or upregulates the expression of genes that suppress atherosclerosis promotion in atherosclerotic Nanomatrix vascular sheets can be identified in the disclosed methods. In some aspects, a gene that promotes atherosclerosis progression can be, but is not limited to, IL-1β, TNF-α, LDLR, IFNγ, IL-6, IL-12, IL-23, IL-18, IL-13, MCP-1, VCAM-1, ICAM-1, PCSK9, CD68, CD14, or CD36. In some aspects, a gene that suppresses atherosclerosis progression can, but is not limited to, CETP, ABCA1, APOE, IL-10, or IL-4. In some aspects, expression levels of genes can be detected and in some aspects, the levels of protein associated with atherosclerosis can be detected. For example, in some aspects a protein associated with atherosclerosis can be, but is not limited to, matrix metalloproteinases (MMP-2, MMP-9, and MMP-3), lipoprotein-associated phospholipase A2, cyclooxygenase, IL-8, IL-1β, TNF-α, IL-6, IL-12, IL-18, IFN-γ, IL-23, IL-13, IL-10, IL-5, or MCP-1. In some aspects, the change in amount of gene or protein that promotes atherosclerosis progression is a decrease in the gene or protein. In some aspects, the change in amount of gene or protein that suppresses atherosclerosis progression is an increase in the gene or protein.

In some aspects, the culturing comprises a culture medium and therapeutic candidate In some aspects, the culture duration is in the range of 1-24 hours, 1-7 day, or 1 week to 12 weeks. In some aspects, a therapeutic drug candidate can be dissolved using culture medium. The therapeutic drug candidate can then be added to an atherosclerotic nanomatrix vascular sheet with culture medium. In some aspects, a therapeutic drug candidate can be used at concentration ranges from nM to uM. In some aspects, the culture medium is selected from culture medium 4, 5, 6, or 7.

In some aspects of the disclosed methods, the atherosclerotic nanomatrix vascular sheet comprises an inflammatory nanomatrix vascular sheet, a plaque, or both. In some aspects, an inflammatory nanomatrix vascular sheet refers to the trilayer Nanomatrix vascular sheet comprising an inflammatory nanomatrix fibroblast sheet, a layer of inflammatory fibroblast, an inflammatory nanomatrix SMC sheet, a layer of inflammatory SMCs, and a layer of dysfunctional ECs. In some aspects, the inflammatory nanomatrix fibroblast sheet comprises nanomatrix and inflammatory fibroblasts, wherein the inflammatory nanomatrix SMC sheet comprises nanomatrix and inflammatory SMCs. In some aspects, the nanomatrix fibroblast sheet and the nanomatrix SMC sheet become inflammatory nanomatrix fibroblast sheet and inflammatory nanomatrix SMC sheets once inducing atherosclerosis in the Nanomatrix vascular sheets. In some aspects, a plaque comprises one or more components, wherein the one or more components can be, but are not limited to, human monocyte, human macrophage, human EC, human fibroblast, human SMC, human foam cell, human low-density lipoproteins, cytokine, chemokine, reactive oxygen species, and/or calcification.

In some aspects, a plaque refers to the presence of components such as human monocytes, human macrophages, human ECs, human fibroblasts, human SMCs, human foam cells, human low-density lipoproteins, cytokines, chemokines, reactive oxygen species, or calcification.

In some aspects, a therapeutic candidate can be any substance that has the potential to cure, mitigate or prevent atherosclerosis. In some aspects, a therapeutic candidate includes, but is not limited to, commercially available drugs, naturally occurring substances, synthesized chemical compounds, biological agents, lipids, proteins, peptides, antibodies, polysaccharides, nucleic acids.

In some aspects, after identifying a therapeutic using the disclosed methods of screening, the drug can then be administered to a subject having atherosclerosis. In some aspects, a therapeutic candidate can work by interacting with specific receptors, enzymes, or other molecular targeting in the body to produce a desired therapeutic effect for atherosclerosis. In some aspects, a therapeutic candidate can be loaded into therapeutic delivery carriers. The therapeutic delivery carriers include but not limited to nanogel, polymeric particles liposomes, micelles, dendrimers, and inorganic nanoparticles. In some aspects, the therapeutics come in various forms, including tablets, capsules, injectables, patches, creams, gels, solutions, suspensions, and powders.

As used herein, the term “therapeutic delivery carriers” refer to materials or systems that are designed to transport a therapeutic to specific sites within the body, with the goal of enhancing its therapeutic efficacy and reducing potential side effects. These carriers can take the form of simple drug molecules or more complex materials such as liposomes, nanoparticles, micelles, and dendrimers. They can be engineered to target specific cells or tissues, to release drugs in a controlled and sustained manner, or to protect the therapeutic from degradation or clearance by the body's immune system. The use of these carriers can improve drug delivery, increase drug stability, and enable more precise and effective treatment strategies for a range of diseases and medical conditions.

iv. The Method for Making Layered Construct Comprising Multiple Nanomatrix Cell Sheets.

Disclosed are methods of making a layered construct comprising multiple nanomatrix cell sheets comprising step 1: producing a first nanomatrix cell sheet by combining cells with a peptide amphiphile solution, applying a layer of peptide amphiphile solution containing cells over a substrate, crosslinking the peptide amphiphiles; step 2: culturing the nanomatrix cell sheet; step 3: seeding cells on the first nanomatrix cell sheet; step 4: producing a second nanomatrix cell sheet on top of the first nanomatrix cell sheet with seeded cells to form a layered construct; step 5: culturing the layered construct; step 6: seeding cells on the top of the layered structure construct; step 7: culturing the layered construct with seeded cells; step 8: producing a third nanomatrix cell sheet on the layered construct; repeating steps 5-8 until a specific number of nanomatrix cell sheet layers is produced in the construct. In some aspects, the number of nanomatrix cell sheet layers at least 1. In some aspects, the cells for producing a nanomatrix cell sheet can be the same or different. In some aspects, the cells for seeding can be the same or different. In some aspects, the cells for seeding can be the same as or different from the cells for producing the nanomatrix cell sheet. In some aspects, the substrate size is at least 0.1 cm². In some aspects, the cell density for producing each nanomatrix cell sheet is in a range between 0.05 million cells/cm² to 5 million cells/cm². In some aspects, the cell density of cell seeding is in a range 0.01 million cells/cm² to 2 million cells/cm². In some aspects, each nanomatrix cell sheet is one layer of the construct. In some aspects, the cells can be from, but are not limited to, artery, vein, skin, kidney, brain, intestine, liver, or lung.

### D. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the nanomatrix vascular sheets, nanomatrix cell sheets, compositions, cells, or combinations thereof described herein.

The disclosed kits can also include directions for making the nanomatrix vascular sheets.

### Examples

### A. Example 1: Fabrication of a Single-Layer Nanomatrix Vascular Sheet (VS)

Experimental: The hAAF sheet was fabricated by encapsulating 2.5 million hAAFs/cm² into 1 wt % PA with vapor from a 0.1M CaCl₂) solution for 1 minute, as shown in Figure TA. Subsequently, the hAAF sheet was cultured in hAAF complete expansion medium with 20% FBS and 1% penicillin-streptomycin for 7 days to allow for cell spreading and remodeling. A similar approach was used to fabricate single-layer nanomatrix hAoSMC and hAEC sheets, which hAoSMC and hAEC were cultured in complete expansion medium supplemented with 20% FBS and 150 μg/mL ascorbic acid, respectively. All single-layer nanomatrix VS were cultured in a humidified tissue culture incubator maintained at 37° C., 5% CO₂, and 20% O₂.

Results: The findings showed that, regardless of the cell types, the vascular cells in the 1 wt % PA nanomatrix exhibited an elongated morphology with appropriate cell spreading on day 7, as depicted in FIG. 1C-D (first column). Moreover, it was demonstrated that an integrated sheet could be formed as early as one day after its fabrication for all three cell types (FIG. 1B). These sheets became more robust and mature after 7-day culture and showed excellent viability (FIG. 1C-E, middle column). It was also confirmed that the cells within the sheet maintained their phenotypes through the immunostaining of typical phenotype markers for hAAF (S100A4) and hAoSMC (SMA-a) (FIG. 1C-D, last column). Furthermore, the DAF-FM staining showed active nitric oxide secretion from hAECs (FIG. 1E, last column), indicating the proper endothelial function of the hAECs of the VS.

### B. Example 2: Fabrication of a Double-Layer Nanomatrix Vascular Sheet (VS)

Experimental: A single-layered hAAF nanomatrix cell sheet was successfully fabricated using the process outlined in Example 1. To construct a double-layered hAAF-hAoSMC nanomatrix cell sheet, hAAFs were seeded on the hAAF sheet and allowed to culture for six hours. Following this, a single-layer hAoSMC nanomatrix cell sheet was fabricated and deposited on top of the hAAF-seeded hAAF nanomatrix cell sheet using a similar method as in Example 1. The resulting hAAF-hAoSMC nanomatrix cell sheet was then cultured for seven days using complete hAoSMC and hAAF expansion medium (11) supplemented with 20% FBS and 150 μg/mL ascorbic acid for cell spreading and remodeling. All double-layer nanomatrix VS were cultured in a humidified tissue culture incubator maintained at 37° C., 5% CO₂, and 20% O₂.

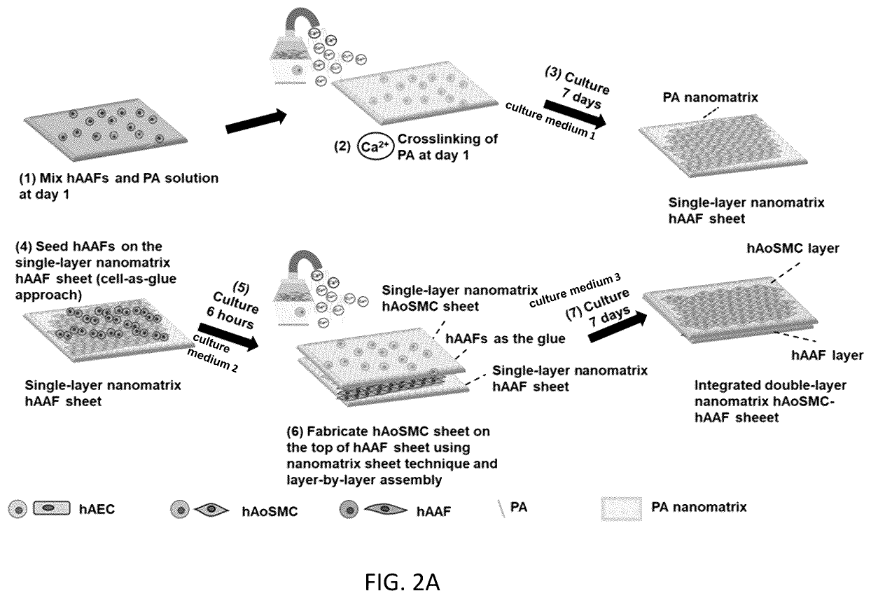

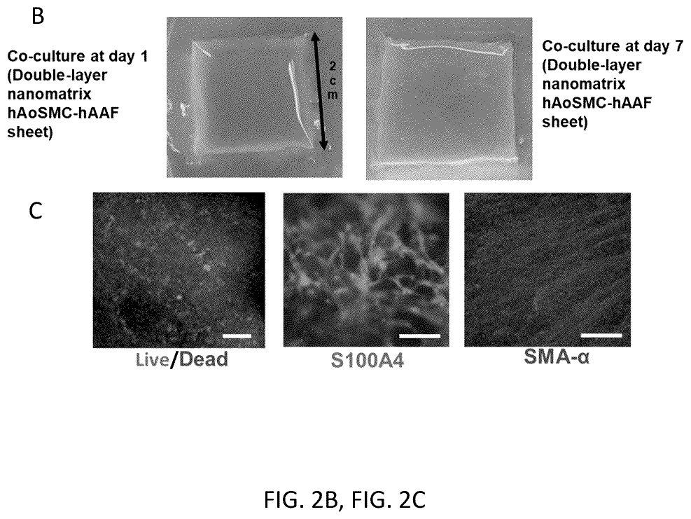

Results: To mimic the discrete vascular layered structure with adventitia and media, a layer-by-layer assembly method was used where a single-layer hAAF sheet was fabricated first, followed by the fabrication and deposition of a single-layer hAoSMC sheet on the hAAF sheet (FIG. 2A). A cell-as-glue approach was developed to promote the integration of the two single-layer nanomatrix VSs into one integrated double-layer VS, taking advantage of the cell-adhesive ligands of PA. Specifically, hAAFs were used as glue and seeded on the hAAF sheet prior to sheet fabrication (FIG. 2A). The hypothesis was that the hAAFs between the two single-layer nanomatrix VSs could bind the cell adhesive ligands from both sheets, thus gluing the two sheets to form an integrated double-layer VS. As expected, a firmly integrated hAoSMC-hAAF sheet was obtained after one day of co-culture, and it maintained its integrity after 7-day culture (FIG. 2B). In contrast, without hAAF seeding (FIG. 2A, step 4), the two single-layer nanomatrix VSs failed to integrate into one sheet, because of a detached and shrinking hAoSMC sheet formed on the hAAF sheet. Furthermore, the encapsulated hAoSMCs and hAAFs within the hAoSMC-hAAF sheet also maintained excellent viability and their respective phenotypes, similar to the cells in the single-layer nanomatrix VSs (FIG. 2C).

### C. Example 3: Fabrication of a Three-Layer Nanomatrix Vascular Sheet (VS)

Experimental: The hAAF-hAoSMC nanomatrix cell sheet was used to fabricate the three-layer nanomatrix VS. hAoSMCs were seeded on the hAAF-hAoSMC sheet (0.1 million hAoSMC/cm²) and cultured for one day. Then, hAECs were directly seeded on the hAoSMC-seeded hAAF-hAoSMC sheet (1 million/cm²) and cultured for an additional 7 days in complete hAoSMC, hAAF, and hAEC medium (1:1:1) supplemented with 20% FBS and 150 μg/mL ascorbic acid to promote cell spreading, remodeling, and endothelialization (FIG. 3B). All three-layer nanomatrix VSs were cultured in a humidified tissue culture incubator maintained at 37° C., 5% CO₂, and 20% O₂.

Results: To create the inner layer of the three-layer nanomatrix VS, direct seeding of hAECs on the hAAF-hAoSMC sheet was used, mimicking the human arterial endothelium's monolayer of ECs. A three-layer VS was fabricated using hAAFs for the bottom layer, RFP-hAoSMCs for the middle layer, and GFP-hAECs for the top layer. This three-layer nanomatrix VS was engineered to mimic the three-layer structure of the vessel wall (FIG. 3A). Notably, no penetration of GFP-hAECs was observed when RFP-hAoSMC seeding occurred before GFP-hAEC seeding. Using this approach, a well-defined three-layer nanomatrix VS was obtained, with hAAF (tunica adventitia), RFP-hAoSMC (tunica media), and GFP-hAEC (tunica intima) layers, from bottom to top, respectively (FIG. 3B). Importantly, the top layer of the VS achieved excellent endothelialization and expressed proper endothelial phenotype, CD31 (FIG. 3C).

### D. Example 4: Fabrication of a Three-Layer Nanomatrix Vascular Sheet with Atherosclerosis (VSA)

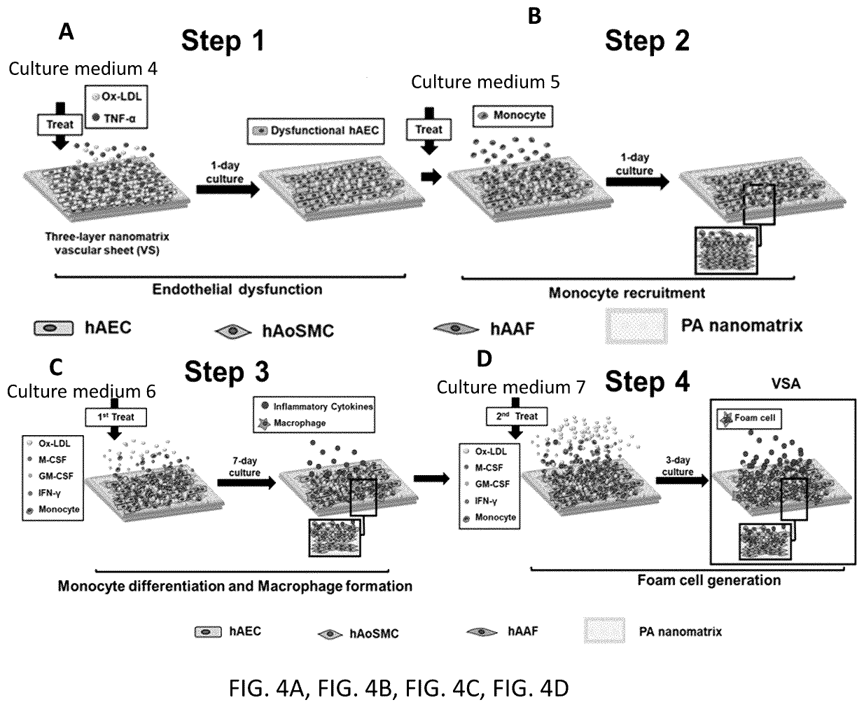

Experimental: The generation of VSA, has four steps, including inducting endothelial dysfunction, monotype recruitment, monocyte differentiation, and macrophage formation, and foam cell generation (FIG. 4A-4C). Endothelial dysfunction was induced using a Dulbecco's modified Eagle's medium (DMEM) (Corning) containing TNF-α (40 ng/mL), and Ox-LDL (50 μg/mL) and 0.5% FBS and 1% penicillin-streptomycin for 1 day (FIG. 4A). The monocyte adhesion was induced via the addition of a DMEM containing U937 monocytes (1 million/mL) and 0.5% FBS, for 1 day (FIG. 4B), and the macrophage formation were induced by adding culture medium 6, comprising a DMEM containing Ox-LDL (50 μg/mL), recombinant human (rh) M-CSF (100 ng/mL), rh GM-CSF (25 ng/mL), rh IFN-γ (100 ng/mL), and 0.5% FBS as well as U937 monocytes (0.1 million/mL) followed by culturing the sheet for 7 days (FIG. 4C). Fresh medium was added into the VSA after 3-day culture. Then, foam cell formation was achieved by adding the culture medium 7 with higher Ox-LDL concentration comprising a DMEM containing Ox-LDL (150 μg/mL), rhM-CSF (100 ng/mL), rhGM-CSF (25 ng/mL), rhIFN-γ (100 ng/mL), 0.5% FBS and U937 monocytes (0.1 million/mL), followed by culturing the sheet for 3 days (FIG. 4D). All culture for induction were cultured in a humified tissue culture incubator maintained at 37° C., 5% CO₂, and 20% O₂.

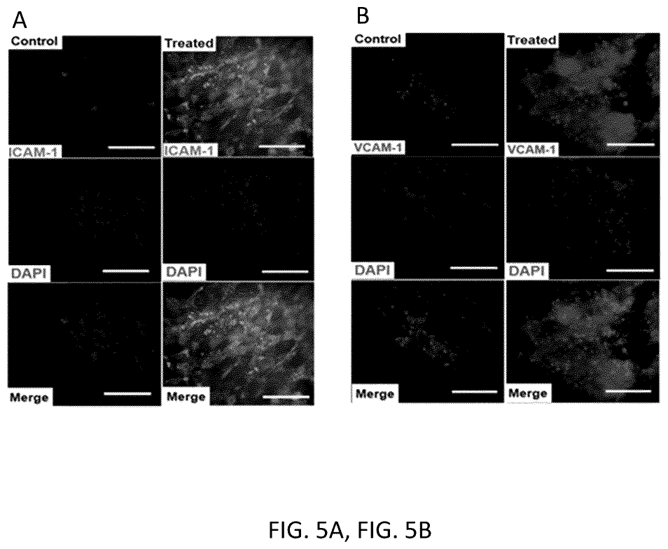

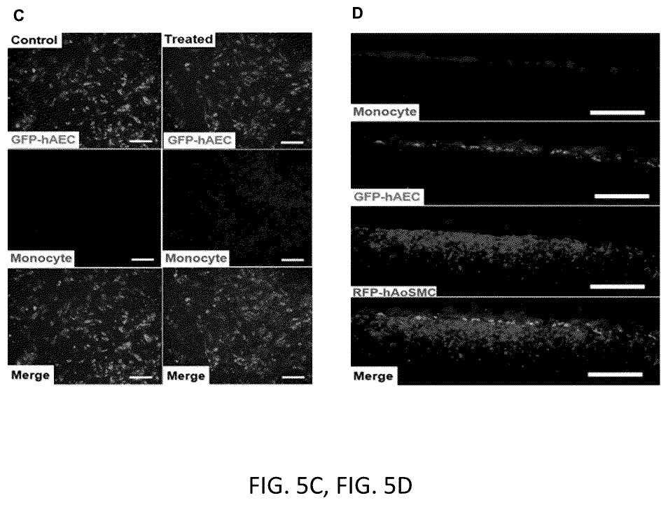

Results: To induce endothelial dysfunction in the VS, a combination of tumor necrosis factor-α TNF-α and Ox-LDL was used. Successful induction of endothelial dysfunction on the VS was demonstrated by a greater expression of endothelial dysfunction markers, ICAM-1, and vascular cell adhesion protein 1 (VCAM-1), compared with the untreated controls (FIG. 5A, B). Additionally, the dysfunctional endothelial layer was observed to recruit more monocytes in great contrast to the untreated VS (FIG. 5C). Furthermore, a cross-section view of the sheet also exhibited a significant number of monocytes (blue) recruited on top of the GFP-hAEC-RFP-hAoSMC-hAAF sheet (FIG. 5D).

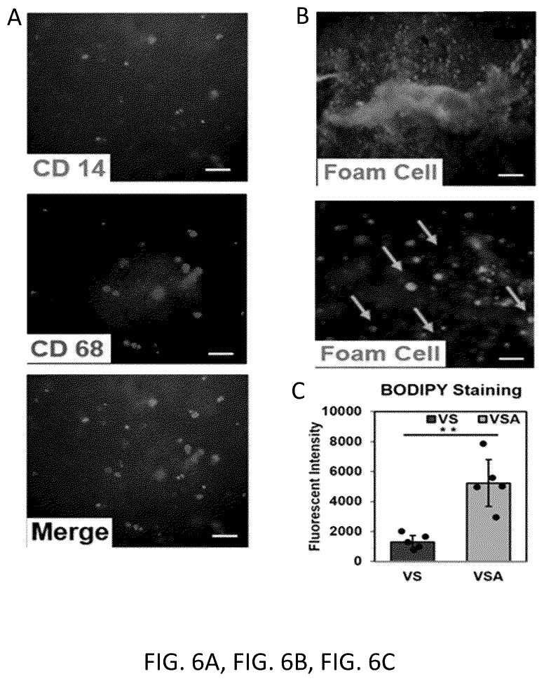

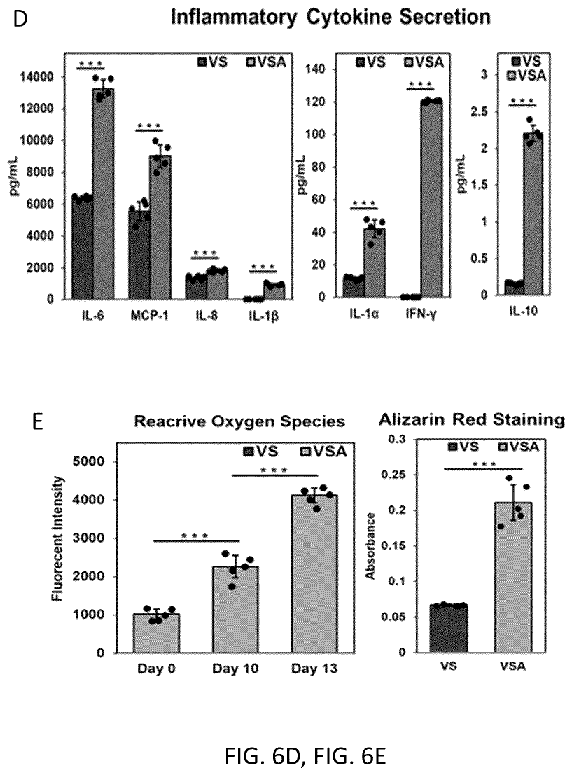

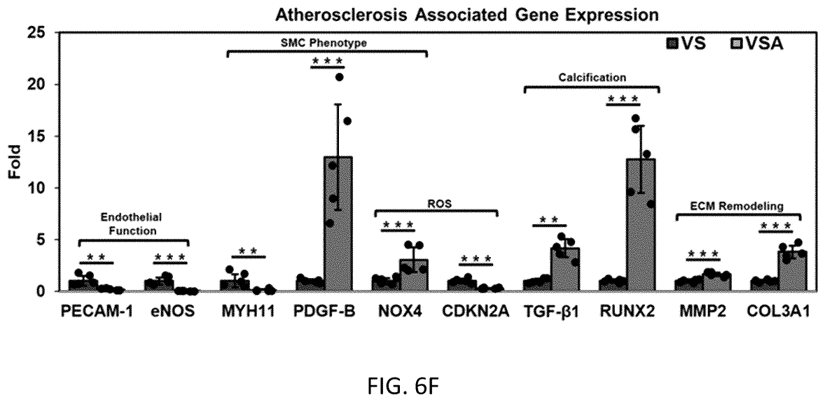

Following endothelial dysfunction and monocyte recruitment, monocyte differentiation into macrophages is critical for promoting inflammatory progression by secreting various pro-inflammatory cytokines and taking up Ox-LDL to form foam cells, following the induction of endothelial dysfunction and monocyte recruitment on the VS, a culture medium 7 and monocytes was employed twice to induce monocytes differentiated into macrophage and the generation of foam cells, thereby forming VSA). Then, the atherosclerotic features of VSA were comprehensively evaluated at the histological level (macrophage marker expression and foam cell generation), molecular level (inflammatory cytokine secretion and oxidative stress level), and genetic level (atherosclerosis-associated gene expression). FIG. 6A shows the VSA demonstrated the positive expression of cluster of differentiation 14 (CD14), a monocyte marker, and cluster of differentiation 68 (CD68), a macrophage marker, expressed by the atherosclerotic macrophages in human plaque, indicating atherosclerotic macrophage formation on the sheet. Notably, as shown in FIG. 6B-C, the boron-dipyrromethene (BODIPY) fluorescent signal of VSA was 4.54±2.02 fold that of the untreated control, indicating a significant amount of foam cells and lipid accumulation induced on the sheet using this approach. Notably, aggregations of foam cells, similar to fat streaks or lipid pools, which is a critical atherosclerotic feature reported in human atherosclerotic plaque, were also observed in the VSA (FIG. 6c). In addition, critical inflammatory cytokines secreted by human atherosclerotic plaque were also produced by the VSA, including interleukin 1α (IL-1α) (3.60±0.51 fold vs. control), IL-1β (44.39±5.32 fold vs. control), monocyte chemoattractant protein-1 (MCP-1) (1.63±0.14 fold vs. control), IFN-γ (354.37±23.11 fold vs. control), and interleukin 6 (IL-6) (2.07±0.072 fold vs. control), and interleukin 8 (IL-8) (1.34±0.076 fold vs. control), indicating an additional key atherosclerotic feature, inflammation, was induced in the VSA (FIG. 6D). In addition to pro-inflammatory cytokines, the secretion of IL-10, an anti-inflammatory cytokine, by the VSA was observed.

Although atherosclerosis is a chronic inflammatory disease, anti-inflammatory cytokines are also generated during inflammatory resections. One typical example is IL-10, found in human advanced atherosclerotic plaque. The expression of both pro-inflammatory and anti-inflammatory cytokines indicated that M1 and M2 macrophages may exist in the VSA. Meanwhile, the dynamic inflammation progression was demonstrated by the reactive oxygen species (ROS) level—an indicator of oxidative stress. It was found that ROS level increased with time during the induction process, with a 2.22±0.41 fold increase from days 0 to 10 and a 1.85±0.24 fold increase from days 10 to 13 (FIG. 6E).

In addition to the molecular level, the atherosclerosis-related gene expression on the VSA was also characterized and classified into five categories: 1) endothelial function, 2) SMC phenotype, 3) inflammation, 4) calcification, and 5) ECM remodeling. As shown in FIG. 6F, compared to the control, the downregulation of platelet endothelial cell adhesion molecule-1 (PECAM-1) (18.31±7.88% vs. control) and endothelial nitric oxide synthase (eNOS) (1.94±0.82% vs. control) indicated the impaired endothelial function of the VSA. Additionally, a decreased expression of SMC contractile phenotype marker, muscle myosin heavy chain 11 (MYH11) (11.58±10.13% vs. control) but an increased expression of platelet-derived growth factor subunit B (PDGF-B) (12.95±5.09 fold vs. control) was observed. SMC phenotype plays a significant role in atherosclerosis-related stenosis, and SMC phenotype transition is a hallmark of atherosclerosis. Normal arterial SMCs maintain their quiescent contractile phenotype, but during atherogenesis, an abundance of PDGF-B is generated, and the SMCs dedifferentiate into a proliferative synthetic phenotype, contributing to atherosclerosis progression and stenosis development. Thus, the decreased MYH11 and increased PDGF-B expression indicates that the SMCs in the VSA may transition from contractile to synthetic phenotypes and atherosclerosis progression in the VSA. Moreover, an upregulation of transforming growth factor-beta 1 (TGF-β1) (4.15±0.88 fold vs. control), nicotinamide adenine dinucleotide phosphate oxidase 4 (NOX4) (3.05±1.17 fold vs. control), and a down-regulation of cyclin-dependent kinase inhibitor 2A (CDKN2A) (29.02±3.90% vs. control) was observed. These markers are related to several crucial pathways associated with atherosclerosis and indicate atherogenesis. For example, the increase of TGF-β1 was reported to lead to fibrotic matrix accumulation, thereby making the ECM susceptible to calcification. TGF-β1 was also reported to induce NOX4 expression in SMCs, which contributes to ROS elevation, agreeing with the results shown previously in FIG. 6E. As reported, CDKN2A is strongly associated with macrophage and monocyte proliferation, the decreased expression of CDKN2A indicates the increased presence of pro-inflammatory monocytes and macrophages on the VSA. The expressions of two genes (matrix metalloproteinases (MMP-2) and collagen type III alpha 1 (COL3A1) associated with ECM remodeling were also evaluated, another feature of atherosclerosis. MMP-2 is strongly related to ECM degradation leading to vulnerable plaque and rupture, resulting in myocardial infarction, while COL3A1 is associated with stenosis and plaque stability. In FIG. 6F, the increased expressions of MMP-2 (1.65±0.17 fold vs. control) and COL3A1 (3.81±0.60 fold vs. control) indicated aggressive ECM remodeling in the VSA. Interestingly, an increased expression of runt-related transcription factor 2 (RUNX2) (12.73±3.25 fold vs. control), a typical early calcification marker, was also noticed, indicating calcification may have initiated on the sheet, which was confirmed by alizarin red staining (ARS) (3.14±0.29 fold vs. control) (FIG. 6E).

In short, using the disclosed methods, VSA was successfully generated with the critical atherosclerosis multi-features, including endothelial dysfunction, monocyte recruitment, macrophage and foam cell generation, cytokine secretion, SMC phenotype transition, calcification initiation, and ECM remodeling.

### E. Example 5. Fabrication and Use of VSAs for Evaluating the Classic Drug (Rosuvastatin and Sirolimus) for Foam Cell Formation

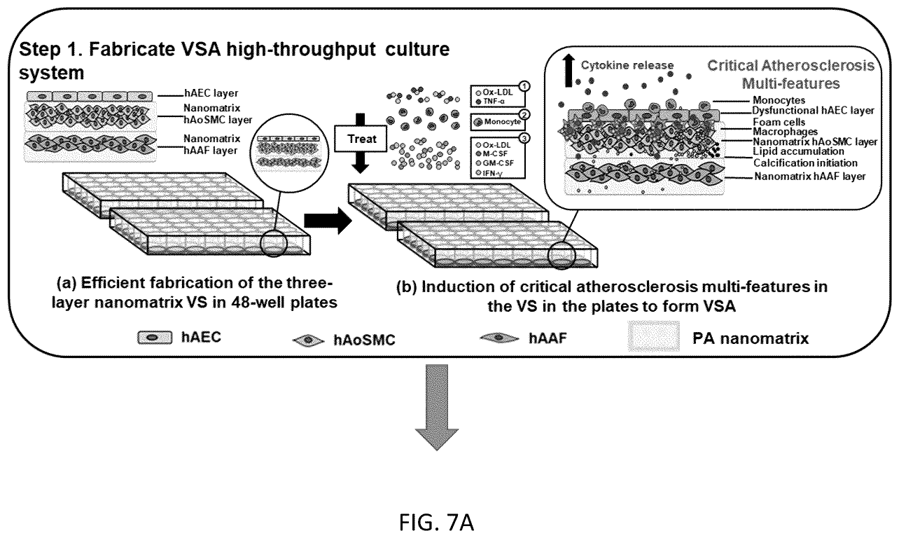

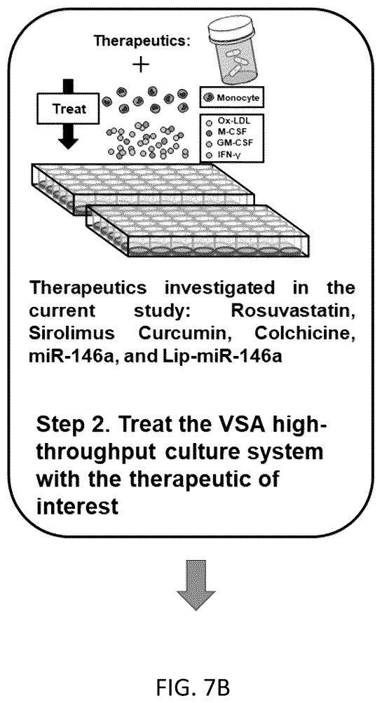

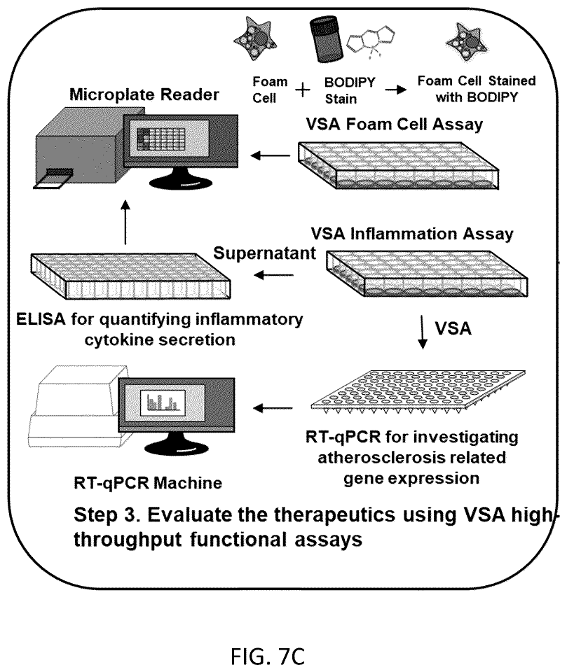

Experimental: (1) Fabrication of large-scale VSA high throughput culture systems: Individual VSAs were fabricated in each well of a 48-well tissue culture plate, following the method described in Example 4 (FIG. 7A, step 1), to create a high throughput culture system for testing drug effects on atherosclerosis. (2) The VSAs were divided into two groups: treated and untreated. (3) The treated group was treated with rosuvastatin (10 uM) or sirolimus (10 nM) by dilution of the stock solution into the “culture medium 7” on day 0 (FIG. 7B, step 2). (4) The VSAs, both treated and untreated, were co-cultured for 10 days with a fresh culture medium 7 with monocytes added on day 3 and day 7, respectively. (5) BODIPY staining was used to quantify foam cell formation: On day 10, a 10 mM BODIPY stock solution was prepared by dissolving in DMSO and stored at −20° C. Before the experiment, a 100 times diluted working solution was prepared from the stock solution using PBS. The VSAs were gently washed with PBS, then BODIPY was added, incubated in the dark for 30 min, imaged immediately using a microscope, and quantified at ex/em 485 nm/520 nm.

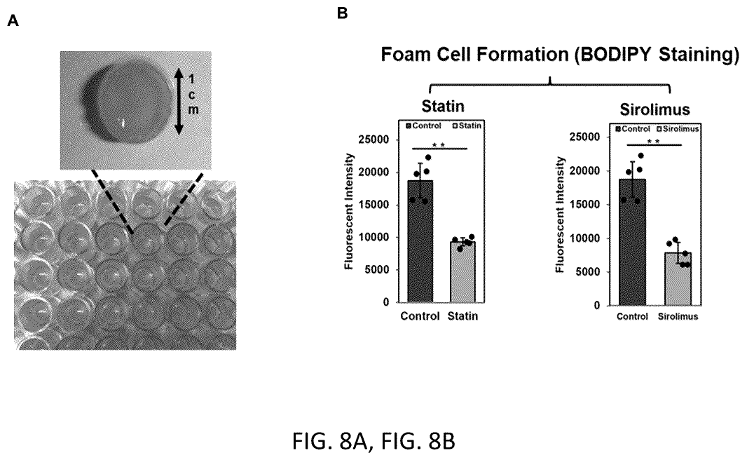

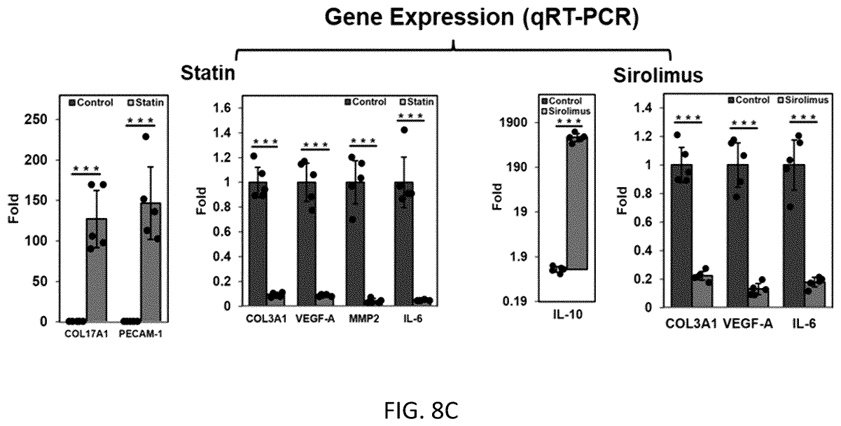

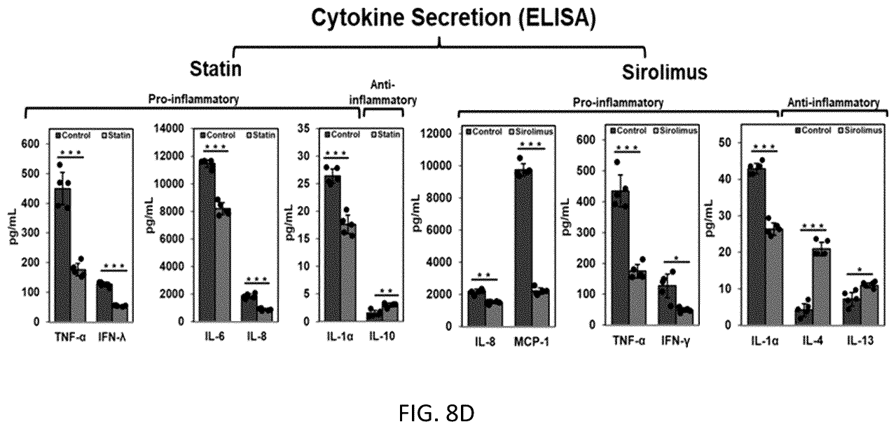

Results: The foam cell assay based on BODIPY-stained VSAs showed that rosuvastatin and sirolimus significantly reduced foam cell formation on the VSA (statin vs. control: 50.77±8.32% vs. 100%, sirolimus vs. control: 42.73±10.62% vs. 100%, FIG. 8B). Additionally, qRT-PCR revealed that rosuvastatin could regulate endothelial function by upregulating the expression of PECAM-1 (146.76±44.63 fold vs. control), indicating its potential for preventing atherosclerosis.

### F. Example 6. Fabrication and Use of VSAs for Evaluating the Classic Drug (Rosuvastatin and Sirolimus) on Atherosclerosis Associated Gene Expression

Experimental: To create a large-scale VSA high throughput culture system, individual VSAs were fabricated in each well of a 48-well tissue culture plate using the method described in Example 4 (FIG. 7A, step 1). The VSAs were then divided into two groups. One group was treated with rosuvastatin (10 uM) and sirolimus (10 nM) by diluting the stock solution into the “culture medium 7” containing monocytes on day 0 (FIG. 7B, step 2). The treated and untreated VSAs were co-cultured for 10 days with fresh culture medium 7 with monocytes added on day 3 and day 7, respectively. On day 10, RNA was extracted using the TRIzol™ reagent (Invitrogen) and purified using the Direct-Zol™ RNA Miniprep Plus kit following the protocol (Zymo Research). EDTA was added before phase separation to bind the calcium and help disintegrate the nanomatrix VSAs. The upper transparent aqueous phase was collected and RNA was suspended in nuclease-free water. The concentration and purity of RNA for each sample were determined using an ND-1000 UV spectrophotometer (Nanodrop). Complementary DNA was synthesized using 50 ng of RNA and reverse transcribed in a 2720 Thermo Cycler (Applied Biosystems) using a Verso cDNA Synthesis Kit (Thermo Fisher) according to the manufacturer's protocol. Samples were prepared in a 96-well PCR plate using the TaqMan Master Mix protocol. Each sample consisted of 2 μL of cDNA solution, 10 μL of 2× master mix, 7 μL of RNA-free water, and 1 μL of gene primer from a TaqMan Gene Expression Assay kit (Applied Biosytems). The PCR plate was run in a LightCycler 480 (Roche Life Science) for the following cycles: pre-incubation at 50° C. for 2 minutes and 95° C. for 10 min; amplification for 45 cycles, at 95° C. for 15 seconds, and 60° C. for 1 minute during each cycle; melting at 95° C. for 5 seconds and 65° C. for 1 minute, and cooling at 40° C. for 30 seconds.

Results: The VSA inflammation gene assay, evaluated through qRT-PCR, revealed that rosuvastatin may suppress intraplaque angiogenesis. The expression of vascular endothelial growth factor A (VEGF-A) was significantly lower in the rosuvastatin-treated group compared to the control. This indicates that rosuvastatin has an inhibitory effect on angiogenesis. Furthermore, the effect of rosuvastatin on extracellular matrix (ECM) remodeling was demonstrated by a 127.03±35.49 fold higher expression of COL17A1 in the rosuvastatin-treated group compared to the control group, indicating an increase in ECM stability. Additionally, there was only a 4.46±1.75% expression of MMP-2 in the rosuvastatin-treated group compared to the control, further supporting the role of rosuvastatin in reducing ECM degradation. Finally, the expression of IL-6 in the rosuvastatin-treated group was only 5.20±0.38% of that in the control group, indicating an anti-inflammatory effect of rosuvastatin.

### G. Example 7. Fabrication and Use of VSAs for Evaluating the Classic Drug (Rosuvastatin and Sirolimus) on Atherosclerosis Associated Cytokine

Experimental: (1) Fabrication of VSA high throughput culture systems: large-scale VSA high throughput culture systems were achieved by fabricating individual VSAs in each well of the 48-well tissue culture plate, following the same method described in Example 4 (FIG. 7A, step 1). (2) Divide the VSAs into two groups. (3) Treatment of VSAs in one group with rosuvastatin (10 uM) or sirolimus (10 nM) by dilution of the stock solution into the culture medium 7 on day 0 (FIG. 7B, step 2). (4) The VSAs, treated or untreated, were culturing in culture medium 7 for 10 days. A fresh culture medium 7 was added to the co-culture system on day 3 and day 7, respectively. (5) ELISA: On day 10, the supernatant from VSAs was collected for ELISA to measure the significant inflammatory cytokine secretion.

Results: Using VSA-based Cytokine Assays, the regulation of several inflammatory cytokines by these two drugs was investigated. Significantly, as indicated in FIG. 8D, rosuvastatin reduced the production of crucial pro-inflammatory cytokines responsible for atherosclerosis-associated inflammation, including IL-1α (66.78±6.52% vs. control), IL-6 (71.50±4.34% vs. control), TNF-α (39.69±5.53% vs. control), IL-8 (48.64±6.17% vs. control), and IFN-γ (44.03±3.20% vs. control). Meanwhile, statin improved the production of the anti-inflammatory cytokine IL-10; 2.10±0.50 fold of secretion in the drug-treated group was observed compared with the control (FIG. 8e). Similarly, the inflammation resolution induced by sirolimus was also detected using the VSA inflammation assay. Specifically, sirolimus reduced pro-inflammatory cytokine secretion, including IL-1α, TNF-α, IL-8, MCP-1, and IFN-γ. Notably, the production for MCP-1, TNF-α, and IFN-γ decreased to 22.76±2.56%, 40.67±6.42%, and 43.64±17.04% of the control group, respectively. Moreover, it was found that sirolimus could improve autophagy-related cytokine secretion (6.42±3.59 fold for interleukin 4 (IL-4) and 1.67±0.59 fold for interleukin 13 (IL-13) relative to the control) (FIG. 8D).

### H. Example 8: Fabrication and Use of VSAs for Evaluating the Classic Drug (Curcumin and Colchicine) for Foam Cell Formation

Experimental: (1) Fabrication of VSA high throughput culture systems: the large-scale VSA high throughput culture system was achieved by fabricating individual VSAs in each well of the 48-well tissue culture plate, following the same method described in Example 4 (FIG. 7A, step 1). (2) Divide the VSAs into two groups. (3) Treatment of VSAs in one group with curcumin (200 uM) or colchicine (100 nM) by dilution of the stock solution into the “culture medium 7” containing monocytes on day 0 (FIG. 7B, step 2). (4) The VSAs, treated or untreated, were co-cultured for 10 days. A fresh culture medium 7 was added to the co-culture system on day 3 and day 7, respectively. (5) BODIPY staining: On day 10, 10 mM BODIPY stock solution was prepared by dissolving in DMSO and stored at −20° C. Before the experiment, 100 times diluted working solution was prepared from stock solution using PBS. Then, the VSAs treated or untreated with curcumin (200 uM), and colchicine (100 nM) were gently washed with PBS, then BODIPY was added and incubated in the dark.

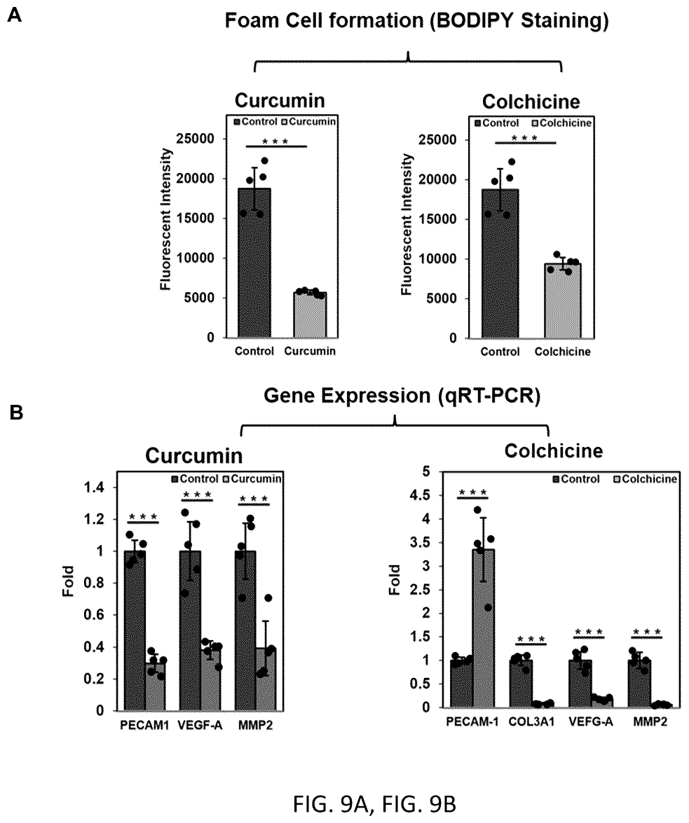

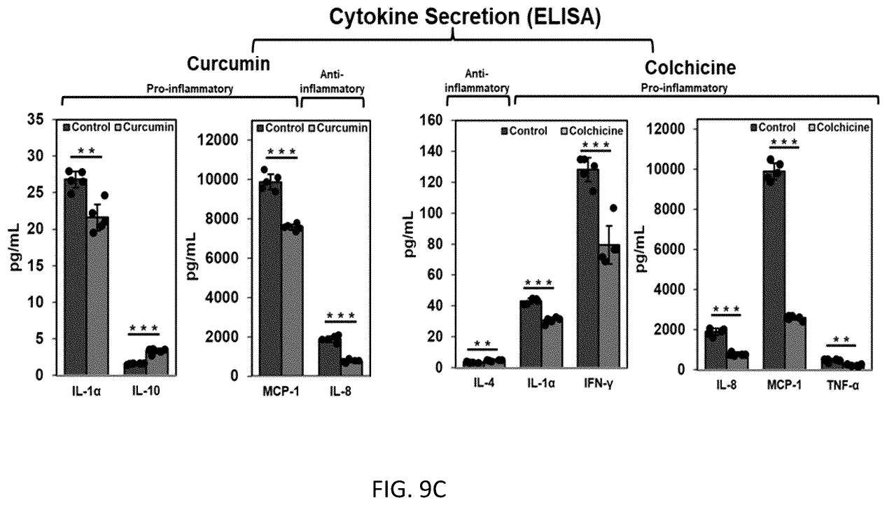

Results: the VSA foam cell assay showed that these two drugs could also reduce foam cell generation significantly (curcumin: 31.23±5.68% vs. control, colchicine: 50.86±4.57% vs. control; FIG. 9A).

### I. Example 9. Fabrication and Use of VSAs for Evaluating the Classic Drug (Curcumin and Colchicine) on Atherosclerosis Associated Gene Expression

Experimental: (1) Fabrication of VSA high throughput culture systems: the large-scale VSA high throughput culture system was achieved by fabricating individual VSAs in each well of the 48-well tissue culture plate, following the same method described in Example 4 (FIG. 7A, step 1). (2) Divide the VSAs into two groups. (3) Treatment of VSAs in one group with curcumin (200 uM) or colchicine (100 nM) by dilution of the stock solution into the “culture medium 7” containing monocytes on day 0 (FIG. 7B, step 2). (4) The VSAs, treated or untreated, were co-cultured for 10 days. A fresh culture medium 7 with monocytes was added to the co-culture system on day 3 and day 7, respectively. (5) RNA extraction and real-time qRT-PCR: On day 10, before phase separation, EDTA (Ethylenediaminetetraacetic acid) was added to bind the calcium and help disintegrate the nanomatrix VSAs. For phase separation, TRIzol™ reagent was added to the samples (>9 times of the sample volume) and incubated for 5 min. Then chloroform was added, mixed by shaking, and centrifuged (11,600 g, 15 min, 4° C.) to obtain phase separation. The upper transparent aqueous phase was collected and purified using the Direct-Zol™ RNA Miniprep Plus kit following the protocol. RNA was suspended in nuclease-free water, and an ND-1000 UV spectrophotometer was used to quantify the concentration and purity of RNA for each sample. Complementary DNA was then synthesized using 50 ng of RNA, which was reverse transcribed in a 2720 Thermo Cycler (Applied Biosystems) using a Verso cDNA Synthesis Kit according to the manufacturer's protocol. Samples were prepared in a 96-well PCR plate using the TaqMan Master Mix protocol. Each sample consisted of 2 μL of cDNA solution, 10 μL of 2× master mix, 7 μL of RNA-free water, and 1 μL of gene primer from a TaqMan Gene Expression Assay kit. The PCR plate was run in a LightCycler 480 for the following cycles: pre-incubation at 50° C. for 2 minutes and 95° C. for 10 min; amplification for 45 cycles, at 95° C. for 15 seconds, and 60° C. for 1 minute during each cycle; melting at 95° C. for 5 seconds and 65° C. for 1 minute, and cooling at 40° C. for 30 seconds.

Results: the results of the VSA gene assay revealed that curcumin participated in the regulation of genes associated with angiogenesis (VEGF-A: 38.01±5.68% vs. control), ECM remodeling (MMP-2: 39.22±7.14% vs. control). Interestingly, although curcumin showed a potential anti-atherosclerotic effect, the assay demonstrated that curcumin might impair endothelial function, as downregulation of PECAM-1 expression was observed in the curcumin-treated group compared to the control (PECAM-1: 29.71±5.68% vs. control) (FIG. 9C). A similar effect of curcumin on the PECAM-1 expression of activated ECs was also reported in the other study. While in contrast, it was noted that colchicine can improve endothelial function by increasing PECAM-1 expression (3.35±0.68 fold vs. control) (FIG. 9B). Additionally, colchicine was shown to regulate ECM remodeling, as demonstrated by decreased COL3A1 (8.93±1.30% vs. control) and MMP-2 (6.36±1.44% vs. control) expression in colchicine-treated VSAs compared to the untreated controls.

### J. Example 10. Fabrication and Use of VSA-based Assays for Evaluating the Potential Drug (Curcumin and Colchicine) on Atherosclerosis Associated Cytokine

Experimental: (1) Fabrication of VSA high throughput culture systems: the large-scale VSA high throughput culture system was achieved by fabricating individual VSAs in each well of the 48-well tissue culture plate, following the same method described in Example 4 (FIG. 7A, step 1). (2) Divide the VSAs into two groups. (3) Treatment of VSAs in one group with curcumin (200 uM) or colchicine (100 nM) by dilution of the stock solution into the culture medium 7 on day 0 (FIG. 7B, step 2). (4) The VSAs, treated or untreated, were cultured using culture medium 7 for 10 days. A fresh culture medium 7 was added to the co-culture system on day 3 and day 7, respectively. (5) ELISA: On day 10, the supernatant from VSAs was collected for ELISA to measure inflammatory cytokine secretion.

Results: The VSA Cytokine Assays also showed that these two drugs could regulate inflammation. For example, curcumin showed an anti-inflammatory effect, as demonstrated by doubling the production of IL-10 (2.10±0.29 fold vs. control) in the VSAs compared with the untreated control and reducing the production of pro-inflammatory cytokines, such as IL-1α (80.58±4.91% vs. control), IL-8 (40.90±5.72% vs. control), and MCP-1 (76.87±3.35% vs. control). Similarly, colchicine showed a potent anti-inflammatory effect via regulating a broad range of inflammatory cytokine secretions. It improved anti-inflammatory activity by increasing the production of IL-4 (1.39±0.14 fold vs. control) while decreasing the production of cytokines, including IL-1α (71.31±5.84%), IL-8 (42.57±1.80% vs. control), MCP-1 (26.31±1.77% vs. control), TNF-α (53.16±7.92% vs. control), and IFN-γ (61.95±7.79% vs. control) (FIG. 9C). Thus, the results from the VSA functional assay indicated that curcumin and colchicine have significant anti-atherosclerosis effects and are worthy of further study.

### K. Example 11: Fabrication and Use of VSAs for Evaluating the Potential Gene Therapy (Free miR-146a and Lip-miR-146a) on Atherosclerosis Associated Cytokine

Experimental: (1) Fabrication of VSA high throughput culture systems: the large-scale VSA high throughput culture system was achieved by fabricating individual VSAs in each well of the 48-well tissue culture plate, following the same method described in Example 4 (FIG. 7A, step 1). (2) Divide the VSAs into two groups. (3) Treatment of VSAs in one group with free miR-146a (600 nM) or Lip-miR-146a (600 nM) by dilution of the stock solution into the culture medium 7 on day 0 (FIG. 7B, step 2). (4) The VSAs, treated or untreated, were co-cultured for 10 days. Afresh culture medium 7 was added to the co-culture system on day 3 and day 7, respectively. (5) ELISA: On day 10, the supernatant from VSAs was collected for ELISA to measure the significant inflammatory cytokine secretion.

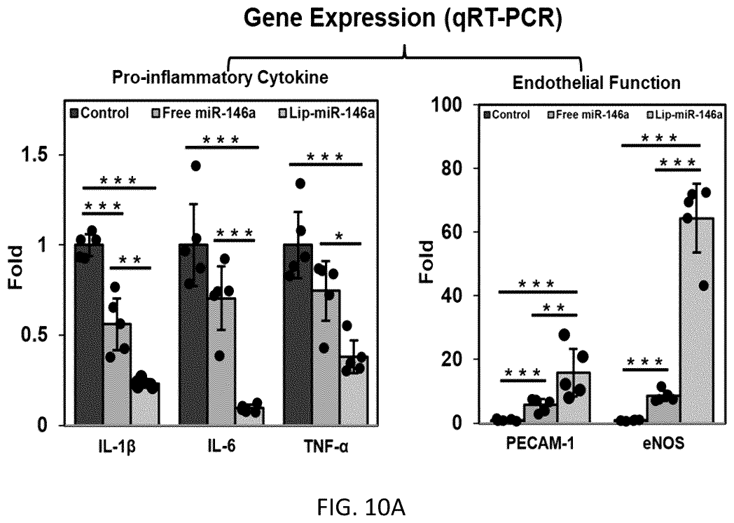

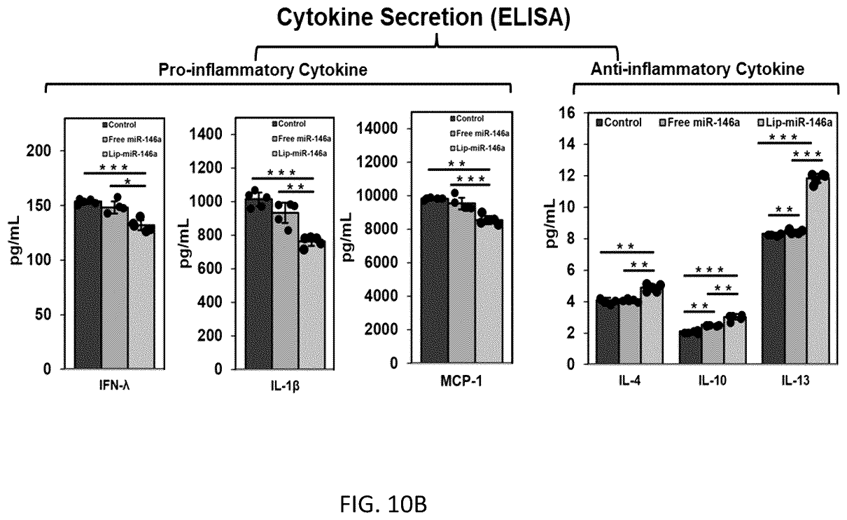

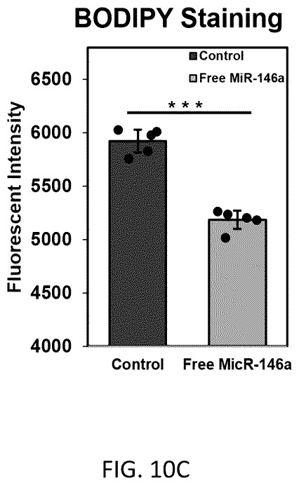

Results: As demonstrated in FIG. 10B, it was found that Lip-miR-146a could reduce the secretion of three pro-inflammatory cytokines that were known to exacerbate atherogenesis (IL-1β: 75.48±5.00% vs. control, IFN-γ: 85.84±3.05% vs. control, MCP-1: 87.04±3.10% vs. control) but increase the production of anti-inflammatory cytokines (IL-10: 1.43±0.10 fold vs. control, IL-13: 1.42±0.04 fold vs. control). In addition, by using the assay, it was found that the efficacy of free miR-146a for inflammation resolution was compromised compared to Lip-miR-146a.

### L. Example 12: Fabrication and Use of VSAs for Evaluating the Potential Gene Therapy (Free miR-146a and Lip-miR-146a) on Atherosclerosis Associated Genes

Experimental: (1) Fabrication of VSA high throughput culture systems: the large-scale VSA high throughput culture system was achieved by fabricating individual VSAs in each well of the 48-well tissue culture plate, following the same method described in Example 4 (FIG. 7A, step 1). (2) Divide the VSAs into two groups. (3) Treatment of VSAs in one group with free miR-146a (600 nM) or Lip-miR-146a (600 nM) by dilution of the stock solution into the “culture medium 7” containing monocytes on day 0 (FIG. 7B, step 2). (4) The VSAs, treated or untreated, were co-cultured for 10 days. A fresh culture medium 7 with monocytes was added to the co-culture system on day 3 and day 7, respectively. (5) RNA extraction and real-time qRT-PCR: On day 10, before phase separation, EDTA (Ethylenediaminetetraacetic acid) was added to bind the calcium and help disintegrate the nanomatrix VSAs. For phase separation, TRIzol™ reagent was added to the samples (>9 times of the sample volume) and incubated for 5 min. Then chloroform was added, mixed by shaking, and centrifuged (11,600 g, 15 min, 4° C.) to obtain phase separation. The upper transparent aqueous phase was collected and purified using the Direct-Zol™ RNA Miniprep Plus kit following the protocol. RNA was suspended in nuclease-free water, and an ND-1000 UV spectrophotometer was used to quantify the concentration and purity of RNA for each sample. Complementary DNA was then synthesized using 50 ng of RNA, which was reverse transcribed in a 2720 Thermo Cycler using a Verso cDNA Synthesis Kit according to the manufacturer's protocol. Samples were prepared in a 96-well PCR plate using the TaqMan Master Mix protocol. Each sample consisted of 2 μL of cDNA solution, 10 μL of 2× master mix, 7 μL of RNA-free water, and 1 μL of gene primer from a TaqMan Gene Expression Assay kit. The PCR plate was run in a LightCycler 480 for the following cycles: pre-incubation at 50° C. for 2 minutes and 95° C. for 10 min; amplification for 45 cycles, at 95° C. for 15 seconds, and 60° C. for 1 minute during each cycle; melting at 95° C. for 5 seconds and 65° C. for 1 minute, and cooling at 40° C. for 30 seconds.

Results: As demonstrated in FIG. 10A, regarding inflammation resolution, at the gene level, the downregulation of inflammatory genes by Lip-miR-146a (IL-1β: 23.30±2.26% vs. control; IL-6: 9.54±1.87% vs. control; TNF-α: 38.14±9.05% vs. control) were observed. The lip-microRNA-146a could improve endothelial function, as demonstrated by the significant upregulation of PECAM-1 and eNOS (PECAM-1: 15.89±7.42 fold vs. control; eNOS: 64.38±10.87 fold vs. control).

### M. Example 13: Fabrication and Use of VSAs for Evaluating the Potential Gene Therapy (Free miR-146a) on Foam Cell Formation

Experimental: (1) Fabrication of VSA high throughput culture systems: the large-scale VSA high throughput culture system was achieved by fabricating individual VSAs in each well of the 48-well tissue culture plate, following the same method described in Example 4 (FIG. 7A, step 1). (2) Divide the VSAs into two groups. (3) Treatment of VSAs in one group with free miR-146a (600 nM) by dilution of the stock solution into the culture medium 7 on day 0 (FIG. 7B, step 2). (4) The VSAs, treated or untreated, were co-cultured for 10 days. Afresh culture medium 7 with monocytes was added to the co-culture system on day 3 and day 7, respectively. BODIPY staining: On day 10, 10 mM BODIPY stock solution was prepared by dissolving in DMSO and stored at −20° C. Before the experiment, 100 times diluted working solution was prepared from stock solution using PBS. Then, the VSAs treated or untreated with free miR-146a were gently washed with PBS, then BODIPY was added and incubated in the dark.

Results: The free miR-146a effect was evaluated using the VSA foam cell assay. Promisingly, the effect of free miR-146a was observed on foam cell generation using the VSA foam cell assay. Compared with control, foam cell generation was suppressed by the free miR-146a (87.55±2.53% vs. control) (FIG. 10C).

### N. Example 14: Fabrication of Single Layer and Double Layer Vein Sheet

Experimental: The vein single layer sheet will be fabricated via the same approach as the fabrication of artery sheet but using human saphenous vein endothelial cells (hSVECs) and human umbilical vein smooth muscle cells (hUVSMCs). Considering the vein has a small number of SMCs in its media layer, the vein single layer sheet has been fabricated to compose a thin vein SMC layer with a few numbers of hUVSMCs (0.5 million hUVSMCs/cm²). Specifically, for making the vein double-layer sheet, hUVSMCs (0.5 million hUVSMCs/cm²) and hSVECs (1 million/cm²) are used.

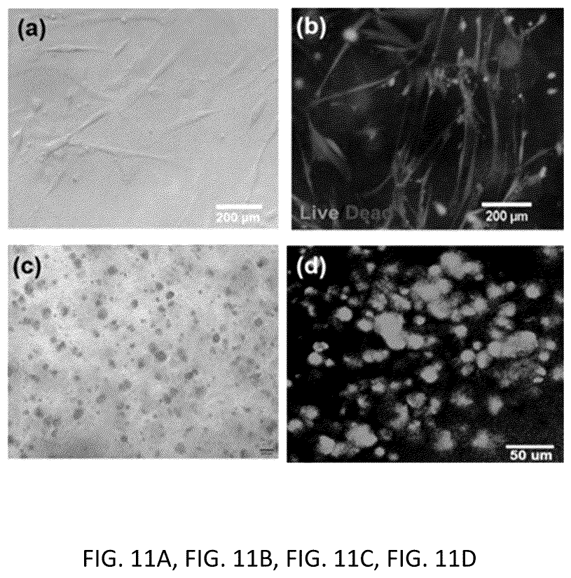

Results: hUVSMC within the sheet showed excellent viability. Furthermore, hSVECs were seeded on the hUVSMC sheet to form hSVEC-hUVSMC sheet (FIG. 11A-B).

The DAF-stained image of hSVECs indicated active nitric oxide secretion from hSVECs (FIG. 11CD), showing the endothelial function of hSVECs on the hSVEC-hUVSMC sheet. The vein cells encapsulated in the nanomatrix thin gel demonstrate excellent viability, cell phenotype markers, cell spreading, and significant ECM production and remodeling after 7 days of static condition.

Those skilled in the art will recognize, or be able to ascertain using no more than routine experimentation, many equivalents to the specific embodiments of the method and compositions described herein. Such equivalents are intended to be encompassed by the following claims.

1. A nanomatrix vascular sheet comprising:
a first layer, wherein the first layer is a nanomatrix cell sheet having fibroblasts,
a second layer, wherein the second layer is a nanomatrix cell sheet having smooth muscle cells (SMCs), and
a third layer, wherein the third layer comprises endothelial cells (ECs).

2. The nanomatrix vascular sheet of claim 1 further comprising:
a layer in between the first layer and second layer, wherein the layer in between the first layer and second layer comprises seeded fibroblasts or seeded SMCs;
a layer in between the second layer and third layer, wherein the layer in between the second layer and third layer comprises seeded SMCs.

3. The nanomatrix vascular sheet of claim 1, wherein the nanomatrix is a hydrogel.

4. The nanomatrix vascular sheet of claim 1, wherein the endothelial cells are human primary endothelial cells.

5. The nanomatrix vascular sheet of claim 4, wherein the human primary endothelial cells are primary human umbilical vein ECs, primary human coronary artery ECs, primary human pulmonary artery ECs, primary human carotid artery ECs, primary human brain microvascular ECs, primary human vein ECs, primary human aortic ECs, mesenchymal stem cell-derived ECs, human induced pluripotent stem cell-derived ECs, or human embryonic stem cell-derived ECs.

6. The nanomatrix vascular sheet of claim 1, wherein the nanomatrix comprises a peptide amphiphile comprising a hydrophobic tail and a hydrophilic peptide sequence.

7. The nanomatrix vascular sheet of claim 6, wherein the peptide amphiphile is (i) PA-GTAGLIGQ-RGDS; (ii) a combination of PA-GTAGLIGQ-RGDS and at least one PA-GTAGLIGQ-YIGSR, PA-GTAGLIGQ-KKKKK-YIGSR, or PA-KKKKK-GTAGLIGQ; (iii) a combination of PA-GTAGLIGQ-RGDS and at least one PA-GTAGLIGQ-KKKKK-YIGSR-NO, or PA-KKKKK-GTAGLIGQ-NO; or (iv) a combination of PA-GTAGLIGQ-RGDS and one or more extracellular matrix molecules.

8. (canceled)

9. (canceled)

10. (canceled)

11. The nanomatrix vascular sheet of claim 6, wherein the peptide amphiphile is-a combination of PA-GTAGLIGQ-RGDS and at least one PA-GTAGLIGQ-KKKKK-YIGSR-NO, or PA-KKKKK-GTAGLIGQ-NO; or a combination of PA-GTAGLIGQ-RGDS and one or more extracellular matrix molecules and wherein the one or more extracellular matrix molecules is collagen, elastin, fibronectin, hyaluronic acid, chondroitin sulfate, heparan sulfate, or proteoglycans.

12. The nanomatrix vascular sheet of claim 1, wherein the nanomatrix vascular sheet is square, circular, or Y shaped.

13. The nanomatrix vascular sheet of claim 1, wherein the smooth muscle cells are primary human umbilical artery SMCs, primary human coronary artery SMCs, primary human pulmonary artery SMCs, primary human carotid artery SMCs, primary human brain vascular SMCs, primary human vein SMCs, primary human aortic SMCs, or human stem cell-derived SMCs.

14. The nanomatrix vascular sheet of claim 1, wherein the fibroblasts are primary human aortic adventitial fibroblasts, primary human coronary artery fibroblasts, primary human cardiac fibroblasts, primary human brain vascular fibroblasts, primary human carotid artery fibroblast, primary pulmonary vein fibroblast, or primary human vein fibroblasts.

15. The nanomatrix vascular sheet of claim 1,
wherein the endothelial cells are dysfunctional,
wherein the fibroblasts are inflammation-activated fibroblasts,
wherein the SMCs are inflammation-activated SMCs, and
wherein the nanomatrix vascular sheet comprises one or more of monocytes, macrophages, foam cells, low-density lipoproteins, cytokines, chemokines, reactive oxygen species, or calcification.

16. The nanomatrix vascular sheet of claim 2,
wherein the endothelial cells are dysfunctional,
wherein the fibroblasts are inflammation-activated fibroblasts,
wherein the SMCs are inflammation-activated SMCs, and
wherein the nanomatrix vascular sheet comprises one or more of monocytes, macrophages, foam cells, low-density lipoproteins, cytokines, chemokines, reactive oxygen species, or calcification.

17. The nanomatrix vascular sheet of claim 15, wherein the cytokines are tumor necrosis factor-alpha (TNF-alpha), interleukin-1 beta (IL-1beta), interleukin-6 (IL-6), interleukin-8 (IL-8), interferon-gamma (IFN-gamma), interleukin-1alpha (IL-1α), or anti-inflammatory cytokines such as interleukin-10 (IL-10), interleukin-4 (IL-4), interleukin-13 (IL-13), or a combination thereof.

18. The nanomatrix vascular sheet of claim 15, wherein the macrophages are differentiated macrophages.

19.-54. (canceled)

55. A method of producing a nanomatrix vascular sheet comprising
a) seeding fibroblasts on a nanomatrix fibroblast sheet;
b) depositing a nanomatrix smooth muscle sheet on top of the seeded fibroblasts of step (a), thereby forming a double-layer nanomatrix cell sheet;
c) seeding smooth muscle cells on the surface of the nanomatrix smooth muscle sheet of the double-layer nanomatrix cell sheet;
d) seeding endothelial cells on top of the smooth muscle cells, thereby forming a trilayer nanomatrix vascular sheet optionally wherein the endothelial cells are combined with a peptide amphiphile solution prior to seeding.

56. The method of claim 55, further comprising culturing the nanomatirx fibroblast sheet after step (a), and prior to step (b), and/or
culturing the double-layer nanomatrix sheet after step (b) and prior step (c), thereby forming a double-layer nanomatrix cell sheet, and/or
culturing the double-layer nanomatrix cell sheet after step (c) and prior to step (d), and/or
culturing the trilayer nanomatrix cell sheet.

57. (canceled)

58. (canceled)

59. (canceled)

60. A method of producing an atherosclerotic nanomatrix vascular sheet comprising:
a) combining fibroblast cells with a peptide amphiphile solution;
b) applying a layer of the peptide amphiphile solution over a substrate;
c) crosslinking the peptide amphiphiles to form a nanomatrix;
d) culturing the nanomatrix with fibroblast cells in a culture medium, thereby forming a nanomatirx fibroblast sheet;
e) seeding fibroblasts on the nanomatrix fibroblast sheet, thereby forming a fibroblast seeded nanomatrix fibroblast sheet;
f) culturing the fibroblast seeded nanomatrix fibroblast sheet in a culture medium;
g) combining smooth muscle cells (SMCs) with a peptide amphiphile solution;
h) applying a layer of the peptide amphiphile solution over the fibroblast seeded nanomatrix fibroblast sheet;
i) crosslinking the peptide amphiphiles;
j) culturing the whole sheet construct in a culture medium, thereby forming double-layer nanomatrix cell sheet;
k) seeding smooth muscle cells on the double-layer nanomatrix cell sheet; thereby forming a smooth muscle cell seeded double-layer nanomatrix cell sheet;
l) culturing the smooth muscle cell seeded double-layer nanomatrix cell sheet in a culture medium;
m) seeding endothelial cells (ECs) with or without peptide amphiphile solution on the smooth muscle cell seeded double-layer nanomatrix cell sheet after culturing in j)
n) culturing the whole sheet construct in a culture medium, thereby forming a triple layer nanomatrix vascular sheet;
o) culturing the triple layer nanomatrix vascular sheet in a culture medium comprising Dulbecco's Modified Eagle Medium (DMEM), TNF-α, Ox-LDL, and serum;
p) culturing the triple layer nanomatrix vascular sheet in a culture medium comprising DMEM, serum, and monocytes;
q) culturing the triple layer nanomatrix vascular sheet in a culture medium comprising DMEM, Ox-LDL, M-CSF, GM-CSF, IFN-γ, serum, and monocytes.

61.-159. (canceled)


SEQUENCE LISTING (4 sequences)

SEQ ID NO: 1 - protein, 8 aa, synthetic construct
GTAGLIGQ

SEQ ID NO: 2 - protein, 4 aa, synthetic construct
RGDS

SEQ ID NO: 3 - protein, 5 aa, synthetic construct
YIGSR

SEQ ID NO: 4 - protein, 5 aa, synthetic construct
KKKKK